(19) United States
(12) Patent Application Publication
(10) Pub. No.: US 2026/0125687 A1
(43) Pub. Date: May 7, 2026

(54) RNA THERAPEUTICS FOR FIBRODYSPLASIA OSSIFICANS PROGRESSIVA AND USES THEREOF

(71) Applicant: UNIVERSITY OF MASSACHUSETTS, Westborough, MA (US)

(72) Inventors: Jae-Hyuck SHIM, Westborough, MA (US); Julia ALTERMAN, Shrewsbury, MA (US); Yeon-Suk YANG, Westborough, MA (US); David COOPER, Framingham, MA (US); Nozomi YAMADA, Westborough, MA (US); Katherine GROSS, Worcester, MA (US); Jillian CAIAZZI, Durham, NC (US)

(21) Appl. No.: 19/350,915

(22) Filed: Oct. 6, 2025

Related U.S. Application Data
(63) Continuation of application No. PCT/US2024/025492, filed on Apr. 19, 2024.
(60) Provisional application No. 63/497,292, filed on Apr. 20, 2023.

(51) Int. Cl. C12N 15/113; A61P 19/08

(52) U.S. Cl. CPC C12N 15/1138; A61P 19/08; C12N 15/1136; C12N 2310/14; C12N 2310/313; C12N 2310/321; C12N 2310/322; C12N 2310/351; C12N 2320/32

(57) ABSTRACT

Aspects of the disclosure relate to compositions and methods for treating Fibrodysplasia ossificans progressiva (FOP). The disclosure is based, in part, on muscle-targeting, chemically-modified interfering nucleic acids (e.g., siRNAs) that bind to and inhibit an ACVR1^{R206H} allele or an Inhba allele of a subject.

### RELATED APPLICATIONS

This application is a continuation of International Patent Application No. PCT/US2024/025492, filed Apr. 19, 2024, which claims priority to U.S. Provisional Patent Application Ser. No. 63/497,292, filed Apr. 20, 2023, the entire contents of which are incorporated herein by reference.

### SEQUENCE LISTING

The instant application contains a Sequence Listing which has been submitted electronically in XML format and is hereby incorporated by reference in its entirety. Said XML file, created on Oct. 6, 2025, is named 769944_UM9-323PCCON_ST26.xml and is 3,016,367 bytes in size.

### BACKGROUND

Fibrodysplasia ossificans progressiva (FOP) is an ultra-rare genetic disorder characterized by progressive, disabling heterotopic ossification (HO) that forms within skeletal muscle, tendons, ligaments, fascia, and aponeuroses. HO develops during flare-ups that occur spontaneously or are triggered by minor trauma, injury, intramuscular injection, or inflammation. Approximately 97% of FOP patients carry a mutation (c.617G>A; R206H) in the ACVR1 gene.

### SUMMARY

Aspects of the disclosure relate to compositions and methods for treating Fibrodysplasia ossificans progressiva (FOP). The disclosure is based, in part, on muscle-targeting, chemically-modified interfering nucleic acids (e.g., siRNAs) that bind to and inhibit an ACVR1^{R206H} allele or an Inhba allele of a subject. In some aspects, the disclosure relates to muscle-targeting, chemically-modified bi-specific siRNAs that bind to and inhibit combinations of genes associated with FOP, for example ACVR1^{206H}, Inhba, IL-1β, TNF, and myostatin (Mstn).

In some aspects, the present disclosure provide an isolated nucleic acid comprising a sense strand and an antisense strand, wherein the antisense strand comprises a sequence that is at least 80% complementary to at least 18 consecutive nucleotides of a ACVR1^{R206H} sequence of SEQ ID NO: 675. In some embodiments, the antisense strand is at least 80% complementary to at least 20 consecutive nucleotide of SEQ ID NO: 675. In some embodiments, antisense strand is at least 80% complementary to the sequence as set forth in any one of SEQ ID NOs: 676-721. In some embodiments, the antisense strand comprises the nucleic acid sequence of any one of SEQ ID NOs: 768-813. 15. In some embodiments, the sense strand comprises the chemical modification pattern set forth in Table 1 or 2. In some embodiments, the antisense strand comprises the chemical modification pattern set forth in Table 1 or 2. In some embodiments, the sense strand comprises the sequence set forth in any one of SEQ ID NOs: 1-16, and 33-62. In some embodiments, the antisense strand comprises the sequence set forth in any one of SEQ ID NOs: 17-32 and 63-92.

In some aspects, the present disclosure provides an isolated nucleic acid comprising a sense strand and an antisense strand, wherein the antisense strand comprises a sequence that is at least 80% complementary to at least 18 consecutive nucleotides of a INHBA sequence of any one of SEQ ID NOs: 817-912. In some embodiments, the antisense strand is at least 80% complementary to at least 20 consecutive nucleotide of any one of SEQ ID NOs: 817-912. In some embodiments, antisense strand is at least 80% complementary to the sequence as set forth in any one of SEQ ID NOs: 913-1007. In some embodiments, the antisense strand comprises the nucleic acid sequence of any one of SEQ ID NOs: 1008-1103. In some embodiments, the sense strand comprises the nucleic acid sequence of any one of SEQ ID NOs: 1104-1199. In some embodiments, the sense strand comprises the chemical modification pattern set forth in Table 3. In some embodiments, the antisense strand comprises the chemical modification pattern set forth in Table 3. In some embodiments, the sense strand comprises the sequence set forth in any one of SEQ ID NOs: 93-188. In some embodiments, the antisense strand comprises the sequence set forth in any one of SEQ ID NOs: 189-284.

In some aspects, the present disclosure provides an isolated nucleic acid comprising a sense strand and an antisense strand, wherein the antisense strand comprises a sequence that is at least 80% complementary to at least 18 consecutive nucleotides of a MSTN sequence of any one of SEQ ID NOs: 1376-1472, and 1200-1287. In some embodiments, the antisense strand is at least 80% complementary to at least 20 consecutive nucleotide of any one of SEQ ID NOs: 1376-1472, and 1200-1287. In some embodiments, antisense strand is at least 80% complementary to the sequence as set forth in any one of SEQ ID NOs: 1473-1569, and 1288-1375. In some embodiments, the antisense strand comprises the nucleic acid sequence of any one of SEQ ID NOs: 1570-1657, and 1746-1842. In some embodiments, the sense strand comprises the nucleic acid sequence of any one of SEQ ID NOs: 1658-1745, and 1843-1939. In some embodiments, the sense strand comprises the chemical modification pattern set forth in Table 4 or 5. In some embodiments, the antisense strand comprises the chemical modification pattern set forth in Table 4 or 5. In some embodiments, the sense strand comprises the sequence set forth in any one of SEQ ID NOs: 285-372 and 481-577. In some embodiments, the antisense strand comprises the sequence set forth in any one of SEQ ID NOs: 373-480 and 578-674.

In some aspects, the disclosure provides an isolated nucleic acid having a length between 10 and 30 nucleotides, comprising a sense strand having the sequence set forth in any one of SEQ ID NOs: 1-16 and 33-62; and an antisense strand having the sequence set forth in any one of SEQ ID NOs: 17-32 and 63-92.

In some aspects, the disclosure provides an isolated nucleic acid having a length between 10 and 30 nucleotides, comprising a sense strand having the sequence set forth in any one of SEQ ID NOs: 93-188; and an antisense strand having the sequence set forth in any one of SEQ ID NOs: 189-284.

In some aspects, the disclosure provides an isolated nucleic acid having a length between 10 and 30 nucleotides, comprising a sense strand having the sequence set forth in any one of SEQ ID NOs: 285-372 and 481-577; and an antisense strand having the sequence set forth in any one of SEQ ID NOs: 373-480 and 578-674.

In some embodiments, a sense strand comprises one or more chemical modifications selected from: one or more ribose modifications, and one or more backbone modifications. In some embodiments, one or more ribose modifications comprise 2′-OMe modifications or 2′-F modifications. In some embodiments, one or more backbone modifications are phosphorothioate (PS) modifications.

In some embodiments, an antisense strand comprises one or more chemical modifications selected from: one or more ribose modifications, and one or more backbone modifications. In some embodiments, one or more ribose modifications comprise 2′-OMe modifications or 2′-F modifications. In some embodiments, one or more backbone modifications are phosphorothioate (PS) modifications.

In some embodiments, an isolated nucleic acid further comprises a cholesterol molecule. In some embodiments, an isolated nucleic acid is conjugated to a cholesterol by a TEG linker.

In some embodiments, an isolated nucleic acid is conjugated to a docosanoic acid (DCA). In some embodiments, DCA is conjugated to an isolated nucleic acid via a cleavable linker.

In some embodiments, a sense strand of an isolated nucleic acid comprises the chemical modification pattern set forth in Table 1 or 2. In some embodiments, an antisense strand of an isolated nucleic acid comprises the chemical modification pattern set forth in Table 1 or 2.

In some embodiments, a sense strand of an isolated nucleic acid comprises the chemical modification pattern set forth in Table 3. In some embodiments, an antisense strand of an isolated nucleic acid comprises the chemical modification pattern set forth in Table 3.

In some embodiments, a sense strand of an isolated nucleic acid comprises the chemical modification pattern set forth in Table 4 or 5. In some embodiments, an antisense strand of an isolated nucleic acid comprises the chemical modification pattern set forth in Table 4 or 5.

In some aspects, the disclosure provides a bi-specific nucleic acid molecule comprising a first siRNA targeting a first RNA transcript selected from ACVR1R206H, Inhba, IL-1β, TNF, and myostatin (Mstn); linked to a second siRNA targeting a second RNA transcript selected from ACVR1R206H, Inhba, IL-1β, TNF, and myostatin (Mstn), wherein the first RNA transcript and second RNA transcript are not transcribed from the same gene.

In some embodiments, a first siRNA and second siRNA are linked via a linking molecule.

In some embodiments, a first siRNA is selected from an siRNA set forth in any one of Tables 1-5.

In some embodiments, a second siRNA is selected from an siRNA set forth in any one of Tables 1-5.

In some embodiments, a first siRNA or second siRNA is conjugated to a docosanoic acid (DCA). In some embodiments, a DCA is conjugated to a first siRNA or second siRNA via a cleavable linker.

In some aspects, the disclosure provides a composition comprising an isolated nucleic acid as described herein, or the bispecific nucleic acid molecule described herein, and a pharmaceutically acceptable excipient.

In some aspects, the disclosure provides a method for inhibiting Activin A activity or function in a subject in need thereof, the method comprising administering to the subject an isolated nucleic acid or bi-specific nucleic acid molecule as described herein.

In some aspects, the disclosure provides a method for treating or preventing heterotopic ossification in a subject in need thereof, the method comprising administering to the subject an isolated nucleic acid or bi-specific nucleic acid molecule as described herein.

In some aspects, the disclosure provides a method for treating or preventing Fibrodysplasia ossificans progressiva (FOP) in a subject in need thereof, the method comprising administering to the subject an isolated nucleic acid or bi-specific nucleic acid molecule as described herein.

In some embodiments, administration comprises local administration. In some embodiments, administration comprises intramuscular injection.

In some embodiments, a subject is a human. In some embodiments, a subject comprises one or more mutations in an ACVR1 allele. In some embodiments, one or more mutations comprise an ACVR1R206H mutation.

### BRIEF DESCRIPTION OF DRAWINGS

FIG. 1 shows a schematic depicting structural features of siRNAs and bi-specific siRNA molecules for use in treatment of FOP.

FIG. 2 shows representative data for testing of allele-specific ACVR1^{R206H} siRNAs in vitro in mouse cells.

FIG. 3 shows representative data for testing of allele-specific ACVR1^{R206H} siRNAs in vitro in human FOP patient-derived iPSCs.

FIG. 4 shows representative data for target gene silencing by SNP6-mismatch mutation siRNAs and SNP8-mismatch mutation siRNAs. Left bars in each group represent ACVR1-WT, and right bar in each group represent ACVR1-R206H.

FIG. 5 shows representative data for dose-response curves of SNP6-based siRNAs.

FIG. 6A shows representative data for SNP6-9 (antisense strand sequence set forth in SEQ ID NO: 69) discrimination between wild-type ACVR1 and ACVR1^{R206H} mRNA in human FOP patient-derived iPSCs.

FIG. 6B shows representative data demonstrating dose-responsive knockdown of human ACVR1^{R206H} protein expression in FOP cells.

FIG. 7 shows representative data demonstrating reduction of Activin A activity after treatment with SNP6-9 siRNA.

FIG. 8 shows representative data demonstrating high transfection efficiency of DCA-conjugated SNP6-9 in HO-causing cell types (e.g., immune cells, chondrocytes, myoblasts/myocytes, etc.).

FIG. 9 shows representative data for screening of mouse Inhba-targeting siRNAs.

FIG. 10 shows representative data for screening of human Inhba-targeting siRNAs.

FIG. 11 shows a schematic depicting representative structures of bi-specific siRNAs targeting genes associated with FOP.

FIG. 12 shows representative data indicating that muscle fibers, chondrocytes within the cartilage analgen, osteoblasts, and osteocytes within heterotopic bone were all transfected with the DCA-siRNAs.

### DETAILED DESCRIPTION

The present disclosure, at least in part, relates to the development of isolated nucleic acids (e.g., siRNA molecules) specifically targeting ACVR1^{R206H} allele (referred to as ACVR1^{R206H} allele-specific siRNA), and the use of such siRNAs for treating Fibrodysplasia ossfficans progressiva (FOP). In some embodiments, an ACVR1^{R206H} allele-specific siRNA described herein specifically targets ACVR1^{R206H} allele without affecting the wild-type ACVR1 allele. In some embodiments, an ACVR1^{R206H} allele-specific siRNA described herein is conjugated to Docosanoic acid (DCA) for efficient and sustainable gene silencing. In some embodiments, an ACVR1^{R206H} allele-specific siRNA comprises one or more modified nucleotides. In some embodiments, an ACVR1^{R206H} allele-specific siRNA can be engineered to be a bi-specific siRNAs that targets ACVR1^{R206H} allele and another molecule that is associated with FOP (e.g., Activin A, INHBA, IL-1β, TNF, and myostatin (Mstn). In some embodiments, the present disclosure also provides siRNA sequences targeting INHBA (e.g., human and/or mouse INHBA) or MSTN (e.g., human and/or mouse MSTN).

Fibrodysplasia ossificans progressiva (FOP, MIM 135100) is a rare autosomal dominant disorder characterized by episodic and progressive heterotopic ossification (HO) of select skeletal muscles, tendons, ligaments, and fascia. The heterotopic bone forms via an endochondral process and is cumulative as it cannot be surgically removed without recurrence. HO in FOP correlated with Bone Morphogenetic Proteins (BMPs) adsorbed into a matrix, which induce the formation of heterotopic bone. Activin Receptor type 1 (ACVR1, MIM 102576), a type I BMP receptor, was identified as the causative gene of FOP. Like all other type I BMP/TGFß receptors, ACVR1 is a single-pass transmembrane protein; its extracellular domain interacts with ligands, whereas its intracellular domain is comprised of a Serine-Glycine-rich region (commonly referred to as the GS domain) and a Serine/Threonine kinase region.

The first and most common FOP-causing variant to be identified was ACVR1^{R206H} arising from a missense mutation, c.617G>A, p.R206H. Other variants were discovered in small subsets of FOP patients but are rare. Activin A was later identified as the ligand responsible for HO in FOP. Activin A specifically activated FOP-variant ACVR1 (e.g., ACVR1^{R206H}) but not wild type ACVR1. Activin A activates ACVR1^{R206H} and results in Smad1/5/8 phosphorylation, mimicking the signal generated when ACVR1 is engaged by BMPs. In contrast, Activin A does not activate wild-type ACVR1-rather, it forms non-signaling complexes (NSCs) with ACVR1 along with its type II receptor partners that can inhibit BMP signaling mediated by wild-type ACVR1. In some cases, patients with FOP are heterozygous, having one allele that harbors the ACVR1^{R206H} mutation, and a wild type AVCR1 allele.

I. siRNAs

In some aspects, the present disclosure provides isolated nucleic acids (e.g., siRNAs) specifically targeting ACVR1^{R206H} (referred to as ACVR1^{R206H} specific siRNA. In some aspects, the present disclosure provides siRNAs targeting Activin A (e.g., a subunit of Activin A Inhibitin, beta A (INHBA), or myostatin (MSTN). In some embodiments, an siRNA described herein mediates RNA interference to reduce expression and/or activity of the target gene.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4):297-310. [0203]Nucleotide analogs may also comprise modifications to the sugar portion of the nucleotides. For example, the 2′ OH— group may be replaced by a group selected from H, OR, R, F, Cl, Br, I, SH, SR, NH2, NHR, NR2, or COOR, wherein R is substituted or unsubstituted Ci-Ce alkyl, alkenyl, alkynyl, aryl, etc. Other possible modifications include those described in U.S. Pat. Nos. 5,858,988, and 6,291,438. The phosphate group of the nucleotide may also be modified, e.g., by substituting one or more of the oxygens of the phosphate group with sulfur (e.g., phosphorothioates), or by making other substitutions, which allow the nucleotide to perform its intended function, such as described in, for example, Eckstein, Antisense Nucleic Acid Drug Dev. 2000 Apr. 10(2): 117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

As used herein, the term “RNA interference” (“RNAi”) refers to a selective intracellular degradation of RNA. RNAi occurs in cells to remove foreign RNAs (e.g., viral RNAs). Natural RNAi proceeds via fragments cleaved from free dsRNA, which direct the degradative mechanism to other similar RNA sequences. RNAi can be induced by engineered molecules, for example an siRNA, to silence the expression of target genes.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g. an orthologue or paralogue) of the target gene. A “target allele” is an allele (e.g., a SNP allele) whose expression is to be selectively inhibited or “silenced.” This silencing can be achieved by RNA silencing, e.g., by cleaving the mRNA of the target gene or target allele by a siRNA. The term “non-target allele” is an allele whose expression is not to be substantially silenced. In certain embodiments, the target and non-target alleles can correspond to the same target gene. In other embodiments, the target allele corresponds to, or is associated with, a target gene, and the non-target allele corresponds to, or is associated with, a non-target gene. In one embodiment, the polynucleotide sequences of the target and non-target alleles can differ by one or more nucleotides. In another embodiment, the target and non-target alleles can differ by one or more allelic polymorphisms (e.g., one or more SNPs). In another embodiment, the target and non-target alleles can share less than 100% sequence identity. The term “polymorphism” as used herein, refers to a variation (e.g., one or more deletions, insertions, or substitutions) in a gene sequence that is identified or detected when the same gene sequence from different sources or subjects (but from the same organism) are compared. For example, a polymorphism can be identified when the same gene sequence from different subjects are compared. Identification of such polymorphisms is routine in the art, the methodologies being similar to those used to detect, for example, breast cancer point mutations. Identification can be made, for example, from DNA extracted from a subject's lymphocytes, followed by amplification of polymorphic regions using specific primers to said polymorphic region. Alternatively, the polymorphism can be identified when two alleles of the same gene are compared. In certain embodiments, the polymorphism is a single nucleotide polymorphism (SNP). A variation in sequence between two alleles of the same gene within an organism is referred to herein as an “allelic polymorphism.” In certain embodiments, the allelic polymorphism corresponds to a SNP allele. For example, the allelic polymorphism may comprise a single nucleotide variation between the two alleles of a SNP. The polymorphism can be at a nucleotide within a coding region but, due to the degeneracy of the genetic code, no change in amino acid sequence is encoded. Alternatively, polymorphic sequences can encode a different amino acid at a particular position, but the change in the amino acid does not affect protein function. Polymorphic regions can also be found in non-encoding regions of the gene. In exemplary embodiments, the polymorphism is found in a coding region of the gene or in an untranslated region (e.g., a 5′ UTR or 3′ UTR) of the gene.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “heterozygosity” refers to the fraction of individuals within a population that are heterozygous (e.g., contain two or more different alleles) at a particular locus (e.g., at a SNP). Heterozygosity may be calculated for a sample population using methods that are well known to those skilled in the art.

As used herein, the term “antisense strand” of an RNA silencing agent, e.g., an siRNA or RNA silencing agent, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA. As used herein, the term “guide strand” refers to a strand of an RNA silencing agent, e.g., an antisense strand of an siRNA duplex or siRNA sequence, that enters into the RISC complex and directs cleavage of the target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, the term “region of complementarity” refers to a nucleotide sequence, e.g., of an oligonucleotide, that is sufficiently complementary to a target region nucleotide sequence, e.g., of target gene mRNA, such that the two nucleotide sequences are capable of annealing to one another under physiological conditions (e.g., in a cell). In some embodiments, a region of complementarity is fully complementary to a nucleotide sequence of target gene mRNA. However, in some embodiments, a region of complementarity is partially complementary to a target nucleotide sequence of the target gene mRNA (e.g., at least 80%, at least 85%, at least 90%, at least 91%, at least 92%, at least 93%, at least 94%, at least 95%, at least 96%, at least 97%, at least 98%, at least 99%, or 100% complementarity). In some embodiments, a region of complementarity contains 1, 2, 3, or 4 mismatches compared with a target nucleotide sequence of target gene mRNA. Complementary, as used herein, refers to the capacity for precise pairing between two nucleotide sequences or two sets of nucleotide sequences. In particular, complementarity is a term that characterizes an extent of hydrogen bond pairing that brings about binding between two nucleotides or two sets of nucleotides. For example, if a base at one position of an oligonucleotide is capable of hydrogen bonding with a base at the corresponding position of target gene mRNA, then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), cytosine-type bases (C) are complementary to guanosine-type bases (G), and universal bases such as 3-nitropyrrole or 5-nitroindole can hybridize to and are considered complementary to any of A, C, G, U, or T. Inosine (I) has also been considered in the art to be a universal base and is considered complementary to any A, C, U, or T.

siRNA, is a class of RNA molecules (e.g., single stranded or double stranded) that target nucleic acids (e.g., mRNAs) for degradation via the RNA interference (RNAi) pathway in cells. In some embodiments, a siRNA is double stranded. The specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective. In some embodiments, the siRNA molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the siRNA molecules are 8 to 30 base pairs in length, 10 to 15 base pairs in length, 10 to 20 base pairs in length, 15 to 25 base pairs in length, 19 to 21 base pairs in length, or 21 to 23 base pairs in length.

Following selection of an appropriate target RNA sequence, siRNA molecules that comprise a nucleotide sequence complementary to all or a portion of the target sequence, i.e., an antisense sequence, can be designed and prepared using suitable methods (see, e.g., PCT Publication Number WO 2004/016735; and U.S. Patent Publication Nos. 2004-0077574 and 2008-0081791; each of which is incorporated herein by reference).

The siRNA molecule can be double-stranded (i.e., a dsRNA molecule comprising an antisense strand and a complementary sense strand) or single-stranded (i.e., a siRNA molecule comprising just an antisense strand). The siRNA molecules can comprise a duplex (i.e. comprising annealed sense and antisense strands with a 3′ overhang), asymmetric duplex (i.e. a duplex with 3′ and 5′ antisense overhangs), hairpin (i.e. when two regions of the same strand, usually complementary in nucleotide sequence when read in opposite directions, base-pair to form a double helix that ends in an unpaired loop), or asymmetric hairpin (i.e. hairpin with a strand overhang) secondary structure, having self-complementary sense and antisense strands. In some embodiments, a siRNA described herein comprising an antisense strand and a sense strand.

In some embodiments, the antisense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths. In some embodiments, the sense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, or 21 to 23 nucleotides in lengths.

i. Human ACVR1^{R206H} Specific siRNAs In some aspects, an siRNA described herein specifically targets human ACVR1^{R206H} nucleic acid sequence (e.g., ACVR1^{R206H} gene and/or ACVR1^{R206H} mRNA). In some embodiments, a target region of an ACVR1^{R206H} specific siRNA comprises the nucleic acid sequence of TCTGGTACAAAGAACAGTGGCTCACCAGATTACACTGTTGGAGTG (SEQ ID NO: 675).

In some embodiments, an ACVR1^{R206H} specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides to an ACVR1^{R206H} target nucleic acid sequence as set forth in SEQ ID NO: 675. In some embodiments, an ACVR1^{R206H} specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides of to an ACVR1^{R206H} target nucleic acid sequence as set forth in SEQ ID NO: 675, except for at least 1, at least 2, at least 3, at least 4, or at least 5 mismatch within the region of complementarity to the nucleotide sequence of SEQ ID NO: 675. Exemplary target sequences for an ACVR1^{R206H} specific siRNA are set forth in Table 6.

Generally, the sequence in any of the Tables in this disclosure could comprises deoxynucleotide or oxynucleotides. In some embodiments, one or more of the thymine bases (T's) in any one of the oligonucleotides provided in any of the Tables in this disclosure may optionally be uracil bases (U's), and/or one or more of the U's may optionally be T's.

TABLE 6
ACVR1^{R206H} Target Sequence

| Name | Target Sequence | SEQ ID NO |
|---|---|---|
| ACVR1_2 | UACAAAGAACAGUGGCUCAC | 676 |
| ACVR1_3 | ACAAAGAACAGUGGCUCACC | 677 |
| ACVR1_4 | CAAAGAACAGUGGCUCACCA | 678 |
| ACVR1_5 | AAAGAACAGUGGCUCACCAG | 679 |
| ACVR1_6 | AAGAACAGUGGCUCACCAGA | 680 |
| ACVR1_7 | AGAACAGUGGCUCACCAGAU | 681 |
| ACVR1_8 | GAACAGUGGCUCACCAGAUU | 682 |
| ACVR1_9 | AACAGUGGCUCACCAGAUUA | 683 |
| ACVR1_10 | ACAGUGGCUCACCAGAUUAC | 684 |
| ACVR1_11 | CAGUGGCUCACCAGAUUACA | 685 |
| ACVR1_12 | AGUGGCUCACCAGAUUACAC | 686 |
| ACVR1_13 | GUGGCUCACCAGAUUACACU | 687 |
| ACVR1_14 | UGGCUCACCAGAUUACACUG | 688 |
| ACVR1_15 | GGCUCACCAGAUUACACUGU | 689 |
| ACVR1_16 | GCUCACCAGAUUACACUGUU | 690 |
| ACVR1_17 | CUCACCAGAUUACACUGUUG | 691 |
| ACVR1_SNP6_2 | AAGAACAGUGGCUCACCAAA | 692 |
| ACVR1_SNP6_3 | AAGAACAGUGGCUCACCUGA | 693 |
| ACVR1_SNP6_4 | AAGAACAGUGGCUCACAAGA | 694 |
| ACVR1_SNP6_5 | AAGAACAGUGGCUCAACAGA | 695 |
| ACVR1_SNP6_7 | AAGAACAGUGGCUAACCAGA | 696 |
| ACVR1_SNP6_8 | AAGAACAGUGGCACACCAGA | 697 |
| ACVR1_SNP6_9 | AAGAACAGUGGAUCACCAGA | 698 |
| ACVR1_SNP6_10 | AAGAACAGUGUCUCACCAGA | 699 |
| ACVR1_SNP6_11 | AAGAACAGUUGCUCACCAGA | 700 |
| ACVR1_SNP6_12 | AAGAACAGAGGCUCACCAGA | 701 |
| ACVR1_SNP6_13 | AAGAACAUUGGCUCACCAGA | 702 |
| ACVR1_SNP6_14 | AAGAACUGUGGCUCACCAGA | 703 |
| ACVR1_SNP6_15 | AAGAAAAGUGGCUCACCAGA | 704 |
| ACVR1_SNP6_16 | AAGUACAGUGGCUCACCAGA | 705 |
| ACVR1_SNP6_17 | AAGAUCAGUGGCUCACCAGA | 706 |
| ACVR1_SNP8_2 | GAACAGUGGCUCACCAGUUU | 707 |
| ACVR1_SNP8_3 | GAACAGUGGCUCACCAGAAU | 708 |
| ACVR1_SNP8_4 | GAAAAGUGGCUCACCAGAUU | 709 |
| ACVR1_SNP8_5 | GAACUGUGGCUCACCAGAUU | 710 |
| ACVR1_SNP8_6 | GAACAUUGGCUCACCAGAUU | 711 |
| ACVR1_SNP8_7 | GAACAGAGGCUCACCAGAUU | 712 |
| ACVR1_SNP8_9 | GAACAGUUGCUCACCAGAUU | 713 |
| ACVR1_SNP8_10 | GAACAGUGUCUCACCAGAUU | 714 |
| ACVR1_SNP8_11 | GAACAGUGGAUCACCAGAUU | 715 |
| ACVR1_SNP8_12 | GAACAGUGGCACACCAGAUU | 716 |
| ACVR1_SNP8_13 | GAACAGUGGCUAACCAGAUU | 717 |
| ACVR1_SNP8_14 | GAACAGUGGCUCAACAGAUU | 718 |
| ACVR1_SNP8_15 | GAACAGUGGCUCACAAGAUU | 719 |
| ACVR1_SNP8_16 | GAACAGUGGCUCACCUGAUU | 720 |
| ACVR1_SNP8_17 | GAACAGUGGCUCACCAUAUU | 721 |

In some embodiments, an ACVR1^{R206H} specific siRNA may be at least 80%, at least 85%, at least 90%, at least 91%, at least 92%, at least 93%, at least 94%, at least 95%, at least 96%, at least 97%, at least 98%, at least 99% or 100% complementary to any one of SEQ ID NOs: 676-721. In some embodiments, a complementary nucleotide sequence need not be 100% complementary to that of ACVR1^{R206H} target sequence to be specifically hybridizable or specific for an ACVR1^{R206H}. In some embodiments, the antisense strand of an ACVR1^{R206H} specific siRNA is fully complementarity with all or a portion of its target sequence. In some embodiments, the antisense strand of an ACVR1^{R206H} specific siRNA includes 1, 2, 3, or more mismatches to the target sequence. In some embodiments, an ACVR1^{R206H} specific siRNA comprises an antisense strand comprising a nucleotide sequence that is complementary (e.g., at least 85%, at least 90%, at least 95%, at least 96%, at least 97%, at least 98%, at least 99%, or 100%) to a target RNA sequence as set forth in any one of SEQ ID NOs: 676-721. In some embodiments, an ACVR1^{R206H} specific siRNA comprises an antisense strand comprising a nucleic acid sequence that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in any one of SEQ ID NOs: 676-721. In some embodiments, an ACVR1^{R206H} specific siRNA comprises an antisense strand comprising a nucleic acid sequence that is complementary the sequence as set forth in any one of SEQ ID NOs: 676-721. Exemplary ACVR1^{R206H} specific siRNA sequences (both antisense and sense strand) are set forth in Tables 7 and 8.

TABLE 7
Exemplary ACVR1^{R206H}
specific siRNA sequences without mismatch

| | | SEQ ID | | SEQ |
|---|---|---|---|---|
| Name | Sense Strand | NO | Antisense Strand | ID NO |
| ACVR1_2 | AAGAACAGUGGCUCAA | 722 | UUGAGCCACUGUUCUUUGUAA | 768 |
| ACVR1_3 | AGAACAGUGGCUCACA | 723 | UGUGAGCCACUGUUCUUUGUA | 769 |
| ACVR1_4 | GAACAGUGGCUCACCA | 724 | UGGUGAGCCACUGUUCUUUGA | 770 |
| ACVR1_5 | AACAGUGGCUCACCAA | 725 | UUGGUGAGCCACUGUUCUUUA | 771 |
| ACVR1_6 | ACAGUGGCUCACCAGA | 726 | UCUGGUGAGCCACUGUUCUUA | 772 |
| ACVR1_7 | CAGUGGCUCACCAGAA | 727 | UUCUGGUGAGCCACUGUUCUA | 773 |
| ACVR1_8 | AGUGGCUCACCAGAUA | 728 | UAUCUGGUGAGCCACUGUUCA | 774 |
| ACVR1_9 | GUGGCUCACCAGAUUA | 729 | UAAUCUGGUGAGCCACUGUUA | 775 |
| ACVR1_10 | UGGCUCACCAGAUUAA | 730 | UUAAUCUGGUGAGCCACUGUA | 776 |
| ACVR1_11 | GGCUCACCAGAUUACA | 731 | UGUAAUCUGGUGAGCCACUGA | 777 |
| ACVR1_12 | GCUCACCAGAUUACAA | 732 | UUGUAAUCUGGUGAGCCACUA | 778 |
| ACVR1_13 | CUCACCAGAUUACACA | 733 | UGUGUAAUCUGGUGAGCCACA | 779 |
| ACVR1_14 | UCACCAGAUUACACUA | 734 | UAGUGUAAUCUGGUGAGCCAA | 780 |
| ACVR1_15 | CACCAGAUUACACUGA | 735 | UCAGUGUAAUCUGGUGAGCCA | 781 |
| ACVR1_16 | ACCAGAUUACACUGUA | 736 | UACAGUGUAAUCUGGUGAGCA | 782 |
| ACVR1_17 | CCAGAUUACACUGUUA | 737 | UAACAGUGUAAUCUGGUGAGA | 783 |

TABLE 8
Exemplary ACVR1^{R206H}
specific siRNA sequences with mismatch

| | | SEQ ID | | SEQ |
|---|---|---|---|---|
| Name | Sense Strand | NO | Antisense Strand | ID NO |
| ACVR1_SNP6_2 | ACAGUGGCUCACCAGA | 738 | UUUGGUGAGCCACUGUUCUUA | 784 |
| ACVR1_SNP6_3 | ACAGUGGCUCACCAGA | 739 | UCAGGUGAGCCACUGUUCUUA | 785 |
| ACVR1_SNP6_4 | ACAGUGGCUCACCAGA | 740 | UCUUGUGAGCCACUGUUCUUA | 786 |
| ACVR1_SNP6_5 | ACAGUGGCUCACCAGA | 741 | UCUGUUGAGCCACUGUUCUUA | 787 |
| ACVR1_SNP6_7 | ACAGUGGCUCACCAGA | 742 | UCUGGUUAGCCACUGUUCUUA | 788 |
| ACVR1_SNP6_8 | ACAGUGGCUCACCAGA | 743 | UCUGGUGUGCCACUGUUCUUA | 789 |
| ACVR1_SNP6_9 | ACAGUGGCUCACCAGA | 744 | UCUGGUGAUCCACUGUUCUUA | 790 |
| ACVR1_SNP6_10 | ACAGUGGCUCACCAGA | 745 | UCUGGUGAGACACUGUUCUUA | 791 |
| ACVR1_SNP6_11 | ACAGUGGCUCACCAGA | 746 | UCUGGUGAGCAACUGUUCUUA | 792 |
| ACVR1_SNP6_12 | ACAGUGGCUCACCAGA | 747 | UCUGGUGAGCCUCUGUUCUUA | 793 |
| ACVR1_SNP6_13 | ACAGUGGCUCACCAGA | 748 | UCUGGUGAGCCAAUGUUCUUA | 794 |
| ACVR1_SNP6_14 | ACAGUGGCUCACCAGA | 749 | UCUGGUGAGCCACAGUUCUUA | 795 |
| ACVR1_SNP6_15 | ACAGUGGCUCACCAGA | 750 | UCUGGUGAGCCACUUUUCUUA | 796 |
| ACVR1_SNP6_16 | ACAGUGGCUCACCAGA | 751 | UCUGGUGAGCCACUGAUCUUA | 797 |
| ACVR1_SNP6_17 | ACAGUGGCUCACCAGA | 752 | UCUGGUGAGCCACUGUACUUA | 798 |
| ACVR1_SNP8_2 | AGUGGCUCACCAGAUA | 752 | UUUCUGGUGAGCCACUGUUCA | 799 |
| ACVR1_SNP8_3 | AGUGGCUCACCAGAUA | 753 | UAACUGGUGAGCCACUGUUCA | 800 |
| ACVR1_SNP8_4 | AGUGGCUCACCAGAUA | 754 | UAUAUGGUGAGCCACUGUUCA | 801 |
| ACVR1_SNP8_5 | AGUGGCUCACCAGAUA | 755 | UAUCAGGUGAGCCACUGUUCA | 802 |
| ACVR1_SNP8_6 | AGUGGCUCACCAGAUA | 756 | UAUCUUGUGAGCCACUGUUCA | 803 |
| ACVR1_SNP8_7 | AGUGGCUCACCAGAUA | 757 | UAUCUGUUGAGCCACUGUUCA | 804 |
| ACVR1_SNP8_9 | AGUGGCUCACCAGAUA | 758 | UAUCUGGUUAGCCACUGUUCA | 805 |
| ACVR1_SNP8_10 | AGUGGCUCACCAGAUA | 759 | UAUCUGGUGUGCCACUGUUCA | 806 |
| ACVR1_SNP8_11 | AGUGGCUCACCAGAUA | 760 | UAUCUGGUGAUCCACUGUUCA | 807 |
| ACVR1_SNP8_12 | AGUGGCUCACCAGAUA | 761 | UAUCUGGUGAGACACUGUUCA | 808 |
| ACVR1_SNP8_13 | AGUGGCUCACCAGAUA | 762 | UAUCUGGUGAGCAACUGUUCA | 809 |
| ACVR1_SNP8_14 | AGUGGCUCACCAGAUA | 763 | UAUCUGGUGAGCCUCUGUUCA | 810 |
| ACVR1_SNP8_15 | AGUGGCUCACCAGAUA | 764 | UAUCUGGUGAGCCAAUGUUCA | 811 |
| ACVR1_SNP8_16 | AGUGGCUCACCAGAUA | 765 | UAUCUGGUGAGCCACAGUUCA | 812 |
| ACVR1_SNP8_17 | AGUGGCUCACCAGAUA | 767 | UAUCUGGUGAGCCACUUUUCA | 813 |

In some embodiments, an ACVR1^{R206H} specific siRNA comprises an antisense strand that comprises at least 15 consecutive nucleosides of (e.g., at least 15, at least 16, at least 17, at least 18, at least 19, or at least 20) the sequence of any one of SEQ ID NOs: 768-813. In some embodiments, an ACVR1^{R206H} specific siRNA further comprises a sense strand that comprises at least 15 consecutive nucleosides (e.g., at least 15, at least 16, at least 17, at least 18, at least 19, or at least 20) complementary to the sequence of any one of SEQ ID NOs: 768-813. In some embodiments, an ACVR1^{R206H} specific siRNA further comprises a sense strand that comprises at least 15 consecutive nucleosides of (e.g., at least 15, at least 16, at least 17, at least 18, at least 19, or at least 20) the sequence of any one of SEQ ID NOs: 722-767.

In some embodiments, an ACVR1^{R206H} specific siRNA comprises an antisense strand that is at least 80% (e.g., at least 85%, at least 90%, at least 95%, at least 96%, at least 97%, at least 98%, at least 99%, or 100%) to the sequence of any one of SEQ ID NOs: 768-813. In some embodiments, an ACVR1^{R206H} specific siRNA further comprises a sense strand that comprises a complementary to a sequence at least 80% (e.g., at least 85%, at least 90%, at least 95%, at least 96%, at least 97%, at least 98%, at least 99%, or 100%) identical to the sequence of any one of SEQ ID NOs: 768-813. In some embodiments, an ACVR1^{R206H} specific siRNA further comprises a sense strand that is at least 80% (e.g., at least 85%, at least 90%, at least 95%, at least 96%, at least 97%, at least 98%, at least 99%, or 100%) identical to the sequence of any one of SEQ ID NOs: 722-767.

In some embodiments, an ACVR1^{R206H} specific siRNA described herein comprises at least one nucleoside modified at the 2′ position of the sugar. In some embodiments, an ACVR1^{R206H} specific siRNA comprises at least one 2′-modified nucleoside. In some embodiments, all of the nucleosides in the oligonucleotide are 2′-modified nucleosides.

In some embodiments, an ACVR1^{R206H} specific siRNA described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside. In some embodiments, an ACVR1^{R206H} specific siRNA comprises one or more 2′-O-methoxyethyl (2′-MOE) modified nucleoside and/or one or more 2′-fluoro (2′-F) modified nucleoside.

In some embodiments, an ACVR1^{R206H} specific siRNA comprises one or more modified one or more modified internucleotide linkages. In some embodiments, the internucleotide linkage is a phosphorothioate linkage. In some embodiments, an ACVR1^{R206H} specific siRNA further comprises a cholesterol molecule. In some embodiments, an ACVR1^{R206H} specific siRNA is conjugated to the cholesterol by a TEG linker. In some embodiments, an ACVR1^{R206H} specific siRNA is conjugated to a docosanoic acid (DCA). In some embodiments, the DCA is conjugated to the isolated nucleic acid via a cleavable linker. Exemplary an ACVR1^{R206H} specific siRNA with chemical modifications are set forth in Tables 1 and 2.

TABLE 1
Modified ACVR1^{R206H} specific siRNA without mismatch

| | | SEQ |
|---|---|---|
| | | ID |
| Name | | NO |
| | Sense Strand | |
| ACVR1_2 | (mA)#(mA)#(mG)(fA)(mA)(fC)(mA)(fG)(mU)(fG)(mG)(mC)(mU)(fC)#(mA)#(mA)-TegChol | 1 |
| ACVR1_3 | (mA)#(mG)#(mA)(fA)(mC)(fA)(mG)(fU)(mG)(fG)(mC)(mU)(mC)(fA)#(mC)#(mA)-TegChol | 2 |
| ACVR1_4 | (mG)#(mA)#(mA)(fC)(mA)(fG)(mU)(fG)(mG)(fC)(mU)(mC)(mA)(fC)#(mC)#(mA)-TegChol | 3 |
| ACVR1_5 | (mA)#(mA)#(mC)(fA)(mG)(fU)(mG)(fG)(mC)(fU)(mC)(mA)(mC)(fC)#(mA)#(mA)-TegChol | 4 |
| ACVR1_6 | (mA)#(mC)#(mA)(fG)(mU)(fG)(mG)(fC)(mU)(fC)(mA)(mC)(mC)(fA)#(mG)#(mA)-TegChol | 5 |
| ACVR1_7 | (mC)#(mA)#(mG)(fU)(mG)(fG)(mC)(fU)(mC)(fA)(mC)(mC)(mA)(fG)#(mA)#(mA)-TegChol | 6 |
| ACVR1_8 | (mA)#(mG)#(mU)(fG)(mG)(fC)(mU)(fC)(mA)(fC)(mC)(mA)(mG)(fA)#(mU)#(mA)-TegChol | 7 |
| ACVR1_9 | (mG)#(mU)#(mG)(fG)(mC)(fU)(mC)(fA)(mC)(fC)(mA)(mG)(mA)(fU)#(mU)#(mA)-TegChol | 8 |
| ACVR1_10 | (mU)#(mG)#(mG)(fC)(mU)(fC)(mA)(fC)(mC)(fA)(mG)(mA)(mU)(fU)#(mA)#(mA)-TegChol | 9 |
| ACVR1_11 | (mG)#(mG)#(mC)(fU)(mC)(fA)(mC)(fC)(mA)(fG)(mA)(mU)(mU)(fA)#(mC)#(mA)-TegChol | 10 |
| ACVR1_12 | (mG)#(mC)#(mU)(fC)(mA)(fC)(mC)(fA)(mG)(fA)(mU)(mU)(mA)(fC)#(mA)#(mA)-TegChol | 11 |
| ACVR1_13 | (mC)#(mU)#(mC)(fA)(mC)(fC)(mA)(fG)(mA)(fU)(mU)(mA)(mC)(fA)#(mC)#(mA)-TegChol | 12 |
| ACVR1_14 | (mU)#(mC)#(mA)(fC)(mC)(fA)(mG)(fA)(mU)(fU)(mA)(mC)(mA)(fC)#(mU)#(mA)-TegChol | 13 |
| ACVR1_15 | (mC)#(mA)#(mC)(fC)(mA)(fG)(mA)(fU)(mU)(fA)(mC)(mA)(mC)(fU)#(mG)#(mA)-TegChol | 14 |
| ACVR1_16 | (mA)#(mC)#(mC)(fA)(mG)(fA)(mU)(fU)(mA)(fC)(mA)(mC)(mU)(fG)#(mU)#(mA)-TegChol | 15 |
| ACVR1_17 | (mC)#(mC)#(mA)(fG)(mA)(fU)(mU)(fA)(mC)(fA)(mC)(mU)(mG)(fU)#(mU)#(mA)-TegChol | 16 |
| | Antisense Strand | |
| ACVR1_2 | P(mU)#(fU)#(mG)(fA)(fG)(fC)(mC)(fA)(mC)(fU)(mG)(fU)(mU)(fC)#(mU)#(fU)#(mU)#(mG)#(mU)#(mA)#(fA) | 17 |
| ACVR1_3 | P(mU)#(fG)#(mU)(fG)(fA)(fG)(mC)(fC)(mA)(fC)(mU)(fG)(mU)(fU)#(mC)#(fU)#(mU)#(mU)#(mG)#(mU)#(fA) | 18 |
| ACVR1_4 | P(mU)#(fG)#(mG)(fU)(fG)(fA)(mG)(fC)(mC)(fA)(mC)(fU)(mG)(fU)#(mU)#(fC)#(mU)#(mU)#(mU)#(mG)#(fA) | 19 |
| ACVR1_5 | P(mU)#(fU)#(mG)(fG)(fU)(fG)(mA)(fG)(mC)(fC)(mA)(fC)(mU)(fG)#(mU)#(fU)#(mC)#(mU)#(mU)#(mU)#(fA) | 20 |
| ACVR1_6 | P(mU)#(fC)#(mU)(fG)(fG)(fU)(mG)(fA)(mG)(fC)(mC)(fA)(mC)(fU)#(mG)#(fU)#(mU)#(mC)#(mU)#(mU)#(fA) | 21 |
| ACVR1_7 | P(mU)#(fU)#(mC)(fU)(fG)(fG)(mU)(fG)(mA)(fG)(mC)(fC)(mA)(fC)#(mU)#(fG)#(mU)#(mU)#(mC)#(mU)#(fA) | 22 |
| ACVR1_8 | P(mU)#(fA)#(mU)(fC)(fU)(fG)(mG)(fU)(mG)(fA)(mG)(fC)(mC)(fA)#(mC)#(fU)#(mG)#(mU)#(mU)#(mC)#(fA) | 23 |
| ACVR1_9 | P(mU)#(fA)#(mA)(fU)(fC)(fU)(mG)(fG)(mU)(fG)(mA)(fG)(mC)(fC)#(mA)#(fC)#(mU)#(mG)#(mU)#(mU)#(fA) | 24 |
| ACVR1_10 | P(mU)#(fU)#(mA)(fA)(fU)(fC)(mU)(fG)(mG)(fU)(mG)(fA)(mG)(fC)#(mC)#(fA)#(mC)#(mU)#(mG)#(mU)#(fA) | 25 |
| ACVR1_11 | P(mU)#(fG)#(mU)(fA)(fA)(fU)(mC)(fU)(mG)(fG)(mU)(fG)(mA)(fG)#(mC)#(fC)#(mA)#(mC)#(mU)#(mG)#(fA) | 26 |
| ACVR1_12 | P(mU)#(fU)#(mG)(fU)(fA)(fA)(mU)(fC)(mU)(fG)(mG)(fU)(mG)(fA)#(mG)#(fC)#(mC)#(mA)#(mC)#(mU)#(fA) | 27 |
| ACVR1_13 | P(mU)#(fG)#(mU)(fG)(fU)(fA)(mA)(fU)(mC)(fU)(mG)(fG)(mU)(fG)#(mA)#(fG)#(mC)#(mC)#(mA)#(mC)#(fA) | 28 |
| ACVR1_14 | P(mU)#(fA)#(mG)(fU)(fG)(fU)(mA)(fA)(mU)(fC)(mU)(fG)(mG)(fU)#(mG)#(fA)#(mG)#(mC)#(mC)#(mA)#(fA) | 29 |
| ACVR1_15 | P(mU)#(fC)#(mA)(fG)(fU)(fG)(mU)(fA)(mA)(fU)(mC)(fU)(mG)(fG)#(mU)#(fG)#(mA)#(mG)#(mC)#(mC)#(fA) | 30 |
| ACVR1_16 | P(mU)#(fA)#(mC)(fA)(fG)(fU)(mG)(fU)(mA)(fA)(mU)(fC)(mU)(fG)#(mG)#(fU)#(mG)#(mA)#(mG)#(mC)#(fA) | 31 |

m is2′-O-methyl nucleosides;
f is 2′-Fluoro nucleosides;
P is 5′-(E)-Vinylphosphonate;
# is phosphorothioate linkage;
TegChol is TEG linker + cholesterol

TABLE 2
Modified ACVR1^{R206H} specific siRNA without mismatch

| | | SEQ |
|---|---|---|
| | | ID |
| Name | | NO |
| | Sense Strand | |
| ACVR1_SNP6_2 | (mA)#(mC)#(mA)(fG)(mU)(fG)(mG)(fC)(mU)(fC)(mA)(mC)(mC)(fA)#(mG)#(mA)-TegChol | 33 |
| ACVR1_SNP6_3 | (mA)#(mC)#(mA)(fG)(mU)(fG)(mG)(fC)(mU)(fC)(mA)(mC)(mC)(fA)#(mG)#(mA)-TegChol | 34 |
| ACVR1_SNP6_4 | (mA)#(mC)#(mA)(fG)(mU)(fG)(mG)(fC)(mU)(fC)(mA)(mC)(mC)(fA)#(mG)#(mA)-TegChol | 35 |
| ACVR1_SNP6_5 | (mA)#(mC)#(mA)(fG)(mU)(fG)(mG)(fC)(mU)(fC)(mA)(mC)(mC)(fA)#(mG)#(mA)-TegChol | 36 |
| ACVR1_SNP6_7 | (mA)#(mC)#(mA)(fG)(mU)(fG)(mG)(fC)(mU)(fC)(mA)(mC)(mC)(fA)#(mG)#(mA)-TegChol | 37 |
| ACVR1_SNP6_8 | (mA)#(mC)#(mA)(fG)(mU)(fG)(mG)(fC)(mU)(fC)(mA)(mC)(mC)(fA)#(mG)#(mA)-TegChol | 38 |
| ACVR1_SNP6_9 | (mA)#(mC)#(mA)(fG)(mU)(fG)(mG)(fC)(mU)(fC)(mA)(mC)(mC)(fA)#(mG)#(mA)-TegChol | 39 |
| ACVR1_SNP6_10 | (mA)#(mC)#(mA)(fG)(mU)(fG)(mG)(fC)(mU)(fC)(mA)(mC)(mC)(fA)#(mG)#(mA)-TegChol | 40 |
| ACVR1_SNP6_11 | (mA)#(mC)#(mA)(fG)(mU)(fG)(mG)(fC)(mU)(fC)(mA)(mC)(mC)(fA)#(mG)#(mA)-TegChol | 41 |
| ACVR1_SNP6_12 | (mA)#(mC)#(mA)(fG)(mU)(fG)(mG)(fC)(mU)(fC)(mA)(mC)(mC)(fA)#(mG)#(mA)-TegChol | 42 |
| ACVR1_SNP6_13 | (mA)#(mC)#(mA)(fG)(mU)(fG)(mG)(fC)(mU)(fC)(mA)(mC)(mC)(fA)#(mG)#(mA)-TegChol | 43 |
| ACVR1_SNP6_14 | (mA)#(mC)#(mA)(fG)(mU)(fG)(mG)(fC)(mU)(fC)(mA)(mC)(mC)(fA)#(mG)#(mA)-TegChol | 44 |
| ACVR1_SNP6_15 | (mA)#(mC)#(mA)(fG)(mU)(fG)(mG)(fC)(mU)(fC)(mA)(mC)(mC)(fA)#(mG)#(mA)-TegChol | 45 |
| ACVR1_SNP6_16 | (mA)#(mC)#(mA)(fG)(mU)(fG)(mG)(fC)(mU)(fC)(mA)(mC)(mC)(fA)#(mG)#(mA)-TegChol | 46 |
| ACVR1_SNP6_17 | (mA)#(mC)#(mA)(fG)(mU)(fG)(mG)(fC)(mU)(fC)(mA)(mC)(mC)(fA)#(mG)#(mA)-TegChol | 47 |
| ACVR1_SNP8_2 | (mA)#(mG)#(mU)(fG)(mG)(fC)(mU)(fC)(mA)(fC)(mC)(mA)(mG)(fA)#(mU)#(mA)-TegChol | 48 |
| ACVR1_SNP8_3 | (mA)#(mG)#(mU)(fG)(mG)(fC)(mU)(fC)(mA)(fC)(mC)(mA)(mG)(fA)#(mU)#(mA)-TegChol | 49 |
| ACVR1_SNP8_4 | (mA)#(mG)#(mU)(fG)(mG)(fC)(mU)(fC)(mA)(fC)(mC)(mA)(mG)(fA)#(mU)#(mA)-TegChol | 50 |
| ACVR1_SNP8_5 | (mA)#(mG)#(mU)(fG)(mG)(fC)(mU)(fC)(mA)(fC)(mC)(mA)(mG)(fA)#(mU)#(mA)-TegChol | 51 |
| ACVR1_SNP8_6 | (mA)#(mG)#(mU)(fG)(mG)(fC)(mU)(fC)(mA)(fC)(mC)(mA)(mG)(fA)#(mU)#(mA)-TegChol | 52 |
| ACVR1_SNP8_7 | (mA)#(mG)#(mU)(fG)(mG)(fC)(mU)(fC)(mA)(fC)(mC)(mA)(mG)(fA)#(mU)#(mA)-TegChol | 53 |
| ACVR1_SNP8_9 | (mA)#(mG)#(mU)(fG)(mG)(fC)(mU)(fC)(mA)(fC)(mC)(mA)(mG)(fA)#(mU)#(mA)-TegChol | 54 |
| ACVR1_SNP8_10 | (mA)#(mG)#(mU)(fG)(mG)(fC)(mU)(fC)(mA)(fC)(mC)(mA)(mG)(fA)#(mU)#(mA)-TegChol | 55 |
| ACVR1_SNP8_11 | (mA)#(mG)#(mU)(fG)(mG)(fC)(mU)(fC)(mA)(fC)(mC)(mA)(mG)(fA)#(mU)#(mA)-TegChol | 56 |
| ACVR1_SNP8_12 | (mA)#(mG)#(mU)(fG)(mG)(fC)(mU)(fC)(mA)(fC)(mC)(mA)(mG)(fA)#(mU)#(mA)-TegChol | 57 |
| ACVR1_SNP8_13 | (mA)#(mG)#(mU)(fG)(mG)(fC)(mU)(fC)(mA)(fC)(mC)(mA)(mG)(fA)#(mU)#(mA)-TegChol | 58 |
| ACVR1_SNP8_14 | (mA)#(mG)#(mU)(fG)(mG)(fC)(mU)(fC)(mA)(fC)(mC)(mA)(mG)(fA)#(mU)#(mA)-TegChol | 59 |
| ACVR1_SNP8_15 | (mA)#(mG)#(mU)(fG)(mG)(fC)(mU)(fC)(mA)(fC)(mC)(mA)(mG)(fA)#(mU)#(mA)-TegChol | 60 |
| ACVR1_SNP8_16 | (mA)#(mG)#(mU)(fG)(mG)(fC)(mU)(fC)(mA)(fC)(mC)(mA)(mG)(fA)#(mU)#(mA)-TegChol | 61 |
| ACVR1_SNP8_17 | (mA)#(mG)#(mU)(fG)(mG)(fC)(mU)(fC)(mA)(fC)(mC)(mA)(mG)(fA)#(mU)#(mA)-TegChol | 62 |
| | Antisense Strand | |
| ACVR1_SNP6_2 | P(mU)#(fU)#(mU)(fG)(fG)(fU)(mG)(fA)(mG)(fC)(mC)(fA)(mC)(fU)#(mG)#(fU)#(mU)#(mC)#(mU)#(mU)#(fA) | 63 |
| ACVR1_SNP6_3 | P(mU)#(fC)#(mA)(fG)(fG)(fU)(mG)(fA)(mG)(fC)(mC)(fA)(mC)(fU)#(mG)#(fU)#(mU)#(mC)#(mU)#(mU)#(fA) | 64 |
| ACVR1_SNP6_4 | P(mU)#(fC)#(mU)(fU)(fG)(fU)(mG)(fA)(mG)(fC)(mC)(fA)(mC)(fU)#(mG)#(fU)#(mU)#(mC)#(mU)#(mU)#(fA) | 65 |
| ACVR1_SNP6_5 | P(mU)#(fC)#(mU)(fG)(fU)(fU)(mG)(fA)(mG)(fC)(mC)(fA)(mC)(fU)#(mG)#(fU)#(mU)#(mC)#(mU)#(mU)#(fA) | 66 |
| ACVR1_SNP6_7 | P(mU)#(fC)#(mU)(fG)(fG)(fU)(mU)(fA)(mG)(fC)(mC)(fA)(mC)(fU)#(mG)#(fU)#(mU)#(mC)#(mU)#(mU)#(fA) | 67 |
| ACVR1_SNP6_8 | P(mU)#(fC)#(mU)(fG)(fG)(fU)(mG)(fU)(mG)(fC)(mC)(fA)(mC)(fU)#(mG)#(fU)#(mU)#(mC)#(mU)#(mU)#(fA) | 68 |
| ACVR1_SNP6_9 | P(mU)#(fC)#(mU)(fG)(fG)(fU)(mG)(fA)(mU)(fC)(mC)(fA)(mC)(fU)#(mG)#(fU)#(mU)#(mC)#(mU)#(mU)#(fA) | 69 |
| ACVR1_SNP6_10 | P(mU)#(fC)#(mU)(fG)(fG)(fU)(mG)(fA)(mG)(fA)(mC)(fA)(mC)(fU)#(mG)#(fU)#(mU)#(mC)#(mU)#(mU)#(fA) | 70 |
| ACVR1_SNP6_11 | P(mU)#(fC)#(mU)(fG)(fG)(fU)(mG)(fA)(mG)(fC)(mA)(fA)(mC)(fU)#(mG)#(fU)#(mU)#(mC)#(mU)#(mU)#(fA) | 71 |
| ACVR1_SNP6_12 | P(mU)#(fC)#(mU)(fG)(fG)(fU)(mG)(fA)(mG)(fC)(mC)(fU)(mC)(fU)#(mG)#(fU)#(mU)#(mC)#(mU)#(mU)#(fA) | 72 |
| ACVR1_SNP6_13 | P(mU)#(fC)#(mU)(fG)(fG)(fU)(mG)(fA)(mG)(fC)(mC)(fA)(mA)(fU)#(mG)#(fU)#(mU)#(mC)#(mU)#(mU)#(fA) | 73 |
| ACVR1_SNP6_14 | P(mU)#(fC)#(mU)(fG)(fG)(fU)(mG)(fA)(mG)(fC)(mC)(fA)(mC)(fA)#(mG)#(fU)#(mU)#(mC)#(mU)#(mU)#(fA) | 74 |
| ACVR1_SNP6_15 | P(mU)#(fC)#(mU)(fG)(fG)(fU)(mG)(fA)(mG)(fC)(mC)(fA)(mC)(fU)#(mU)#(fU)#(mU)#(mC)#(mU)#(mU)#(fA) | 75 |
| ACVR1_SNP6_16 | P(mU)#(fC)#(mU)(fG)(fG)(fU)(mG)(fA)(mG)(fC)(mC)(fA)(mC)(fU)#(mG)#(fA)#(mU)#(mC)#(mU)#(mU)#(fA) | 76 |
| ACVR1_SNP6_17 | P(mU)#(fC)#(mU)(fG)(fG)(fU)(mG)(fA)(mG)(fC)(mC)(fA)(mC)(fU)#(mG)#(fU)#(mA)#(mC)#(mU)#(mU)#(fA) | 77 |
| ACVR1_SNP8_2 | P(mU)#(fU)#(mU)(fC)(fU)(fG)(mG)(fU)(mG)(fA)(mG)(fC)(mC)(fA)#(mC)#(fU)#(mG)#(mU)#(mU)#(mC)#(fA) | 78 |
| ACVR1_SNP8_3 | P(mU)#(fA)#(mA)(fC)(fU)(fG)(mG)(fU)(mG)(fA)(mG)(fC)(mC)(fA)#(mC)#(fU)#(mG)#(mU)#(mU)#(mC)#(fA) | 79 |
| ACVR1_SNP8_4 | P(mU)#(fA)#(mU)(fA)(fU)(fG)(mG)(fU)(mG)(fA)(mG)(fC)(mC)(fA)#(mC)#(fU)#(mG)#(mU)#(mU)#(mC)#(fA) | 80 |
| ACVR1_SNP8_5 | P(mU)#(fA)#(mU)(fC)(fA)(fG)(mG)(fU)(mG)(fA)(mG)(fC)(mC)(fA)#(mC)#(fU)#(mG)#(mU)#(mU)#(mC)#(fA) | 81 |
| ACVR1_SNP8_6 | P(mU)#(fA)#(mU)(fC)(fU)(fU)(mG)(fU)(mG)(fA)(mG)(fC)(mC)(fA)#(mC)#(fU)#(mG)#(mU)#(mU)#(mC)#(fA) | 82 |
| ACVR1_SNP8_7 | P(mU)#(fA)#(mU)(fC)(fU)(fG)(mU)(fU)(mG)(fA)(mG)(fC)(mC)(fA)#(mC)#(fU)#(mG)#(mU)#(mU)#(mC)#(fA) | 83 |
| ACVR1_SNP8_9 | P(mU)#(fA)#(mU)(fC)(fU)(fG)(mG)(fU)(mU)(fA)(mG)(fC)(mC)(fA)#(mC)#(fU)#(mG)#(mU)#(mU)#(mC)#(fA) | 84 |
| ACVR1_SNP8_10 | P(mU)#(fA)#(mU)(fC)(fU)(fG)(mG)(fU)(mG)(fU)(mG)(fC)(mC)(fA)#(mC)#(fU)#(mG)#(mU)#(mU)#(mC)#(fA) | 85 |
| ACVR1_SNP8_11 | P(mU)#(fA)#(mU)(fC)(fU)(fG)(mG)(fU)(mG)(fA)(mU)(fC)(mC)(fA)#(mC)#(fU)#(mG)#(mU)#(mU)#(mC)#(fA) | 86 |
| ACVR1_SNP8_12 | P(mU)#(fA)#(mU)(fC)(fU)(fG)(mG)(fU)(mG)(fA)(mG)(fA)(mC)(fA)#(mC)#(fU)#(mG)#(mU)#(mU)#(mC)#(fA) | 87 |
| ACVR1_SNP8_13 | P(mU)#(fA)#(mU)(fC)(fU)(fG)(mG)(fU)(mG)(fA)(mG)(fC)(mA)(fA)#(mC)#(fU)#(mG)#(mU)#(mU)#(mC)#(fA) | 88 |
| ACVR1_SNP8_14 | P(mU)#(fA)#(mU)(fC)(fU)(fG)(mG)(fU)(mG)(fA)(mG)(fC)(mC)(fU)#(mC)#(fU)#(mG)#(mU)#(mU)#(mC)#(fA) | 89 |
| ACVR1_SNP8_15 | P(mU)#(fA)#(mU)(fC)(fU)(fG)(mG)(fU)(mG)(fA)(mG)(fC)(mC)(fA)#(mA)#(fU)#(mG)#(mU)#(mU)#(mC)#(fA) | 90 |
| ACVR1_SNP8_16 | P(mU)#(fA)#(mU)(fC)(fU)(fG)(mG)(fU)(mG)(fA)(mG)(fC)(mC)(fA)#(mC)#(fA)#(mG)#(mU)#(mU)#(mC)#(fA) | 91 |
| ACVR1_SNP8_17 | P(mU)#(fA)#(mU)(fC)(fU)(fG)(mG)(fU)(mG)(fA)(mG)(fC)(mC)(fA)#(mC)#(fU)#(mU)#(mU)#(mU)#(mC)#(fA) | 92 |

m is2′-O-methyl nucleosides;
f is 2′-Fluoro nucleosides;
P is 5′-(E)-Vinylphosphonate;
# is phosphorothioate linkage;
TegChol is TEG linker + cholesterol

In some embodiments, ACVR1^{R206H} specific siRNA comprises an anti-sense strand that comprises the nucleic acid sequence with chemical modification pattern set forth in Table 1 or 2. In some embodiments, ACVR1^{R206H} specific siRNA comprises an anti-sense strand that comprises the nucleic acid sequence with chemical modification pattern as set forth in SEQ ID NOs: 17-32, and 63-92. In some embodiments, ACVR1^{R206H} specific siRNA comprises a sense strand that comprises the nucleic acid sequence with chemical modification pattern set forth in Table 1 or 2. In some embodiments, ACVR1^{R206H} specific siRNA comprises a sense strand that comprises the nucleic acid sequence with chemical modification pattern as set forth in SEQ ID NOs: 1-16, and 33-62.

ii. Human and Mouse INHBA Specific siRNAs

In some aspects, an siRNA described herein specifically targets INHBA nucleic acid sequence (e.g., INHBA gene and/or INHBA mRNA). In some embodiments, an siRNA described herein specifically targets mouse and/or INHBA nucleic acid sequence (referred to as INHBA specific siRNA). Exemplary nucleic acid sequence encoding human INHBA is set forth in SEQ ID NO: 814:

ATGCCCTTGCTTTGGCTGAGAGGATTTCTGTTGGCAAGTTGCTGGATTATAGTGAGGAGTTCCCCCACCC
CAGGATCCGAGGGGCACAGCGCGGCCCCCGACTGTCCGTCCTGTGCGCTGGCCGCCCTCCCAAAGGATGT
ACCCAACTCTCAGCCAGAGATGGTGGAGGCCGTCAAGAAGCACATTTTAAACATGCTGCACTTGAAGAAG
AGACCCGATGTCACCCAGCCGGTACCCAAGGCGGCGCTTCTGAACGCGATCAGAAAGCTTCATGTGGGCA
AAGTCGGGGAGAACGGGTATGTGGAGATAGAGGATGACATTGGAAGGAGGGCAGAAATGAATGAACTTAT
GGAGCAGACCTCGGAGATCATCACGTTTGCCGAGTCAGGAACAGCCAGGAAGACGCTGCACTTCGAGATT
TCCAAGGAAGGCAGTGACCTGTCAGTGGTGGAGCGTGCAGAAGTCTGGCTCTTCCTAAAAGTCCCCAAGG
CCAACAGGACCAGGACCAAAGTCACCATCCGCCTCTTCCAGCAGCAGAAGCACCCGCAGGGCAGCTTGGA
CACAGGGGAAGAGGCCGAGGAAGTGGGCTTAAAGGGGGAGAGGAGTGAACTGTTGCTCTCTGAAAAAGTA
GTAGACGCTCGGAAGAGCACCTGGCATGTCTTCCCTGTCTCCAGCAGCATCCAGCGGTTGCTGGACCAGG
GCAAGAGCTCCCTGGACGTTCGGATTGCCTGTGAGCAGTGCCAGGAGAGTGGCGCCAGCTTGGTTCTCCT
GGGCAAGAAGAAGAAGAAAGAAGAGGAGGGGGAAGGGAAAAAGAAGGGCGGAGGTGAAGGTGGGGCAGGA
GCAGATGAGGAAAAGGAGCAGTCGCACAGACCTTTCCTCATGCTGCAGGCCCGGCAGTCTGAAGACCACC
CTCATCGCCGGCGTCGGCGGGGCTTGGAGTGTGATGGCAAGGTCAACATCTGCTGTAAGAAACAGTTCTT
TGTCAGTTTCAAGGACATCGGCTGGAATGACTGGATCATTGCTCCCTCTGGCTATCATGCCAACTACTGC
GAGGGTGAGTGCCCGAGCCATATAGCAGGCACGTCCGGGTCCTCACTGTCCTTCCACTCAACAGTCATCA
ACCACTACCGCATGCGGGGCCATAGCCCCTTTGCCAACCTCAAATCGTGCTGTGTGCCCACCAAGCTGAG
ACCCATGTCCATGTTGTACTATGATGATGGTCAAAACATCATCAAAAAGGACATTCAGAACATGATCGTG
GAGGAGTGTGGGTGCTCATAG

Exemplary nucleic acid sequence encoding mouse Inhba is set forth in SEQ ID NO: 815.

ATGCCCTTGCTTTGGCTGAGAGGATTTCTGTTGGCAAGTTGCTGGATTATAGTGAGGAGTTCCCCCACCC
CAGGATCCGAGGGGCACGGCTCAGCCCCGGACTGCCCGTCCTGTGCGCTGGCCACCCTTCCGAAGGATGG
ACCTAACTCTCAGCCAGAGATGGTAGAGGCTGTCAAGAAGCACATCTTAAACATGCTGCACTTGAAGAAG
AGACCCGATGTCACCCAGCCGGTGCCCAAGGCGGCGCTTCTCAACGCGATCAGAAAGCTTCATGTGGGTA
AAGTGGGGGAGAACGGGTATGTGGAGATAGAGGACGACATTGGCAGGAGGGCCGAAATGAATGAACTCAT
GGAGCAGACCTCGGAGATCATCACCTTTGCCGAGTCAGGCACAGCCAGGAAGACACTGCACTTTGAGATT
TCCAAGGAAGGCAGTGACCTGTCAGTAGTGGAGCGTGCAGAAGTGTGGCTCTTCCTGAAAGTCCCCAAGG
CTAACAGAACCAGGACCAAAGTCACCATCCGTCTATTTCAGCAGCAGAAGCACCCACAGGGCAGCTTGGA
CACGGGGGATGAGGCCGAGGAAATGGGCTTAAAGGGGGAGAGGAGTGAACTGTTGCTATCAGAGAAAGTA
GTTGATGCTCGGAAGAGTACCTGGCACATCTTTCCAGTGTCCAGCAGCATCCAGCGCCTGCTGGACCAGG
GAAAGAGTTCCCTGGACGTGCGGATTGCTTGTGAGCAGTGCCAGGAGAGTGGTGCCAGTCTAGTGCTTCT
GGGCAAGAAGAAGAAGAAAGAGGTGGATGGAGATGGGAAGAAGAAAGATGGGAGTGACGGAGGGCTGGAA
GAGGAAAAGGAACAGTCACATAGACCTTTCCTCATGCTGCAGGCTAGGCAGTCCGAAGACCACCCTCATC
GCAGGCGTAGGCGGGGCTTGGAGTGCGACGGCAAGGTCAACATTTGCTGTAAGAAACAGTTCTTTGTCAG
CTTCAAGGACATTGGCTGGAATGACTGGATCATTGCTCCCTCTGGCTATCACGCCAATTATTGTGAGGGG
GAGTGCCCAAGCCACATAGCAGGCACCTCTGGGTCCTCGCTCTCCTTCCACTCAACAGTCATTAACCACT
ACCGCATGAGGGGTCACAGCCCCTTTGCCAACCTTAAGTCATGCTGTGTGCCCACCAAGCTGAGACCCAT
GTCCATGCTGTATTACGATGATGGTCAAAACATCATCAAAAAGGACATTCAAAACATGATTGTGGAGGAG
TGTGGCTGCTCCTAG

Exemplary INHBA target gene region sequences and corresponding target sequences are set forth in Table 9.

TABLE 9
Human INHBA and mouse Inhba target gene regions sequences and target
sequences

| | | SEQ | | SEQ ID |
|---|---|---|---|---|
| Name | Target Sequence | ID NO | Target region | NO |
| INHBA_ | UCAACUAUGUCAAUGGUU | 913 | TTATATGAGTGCATTTCAACTATGT | 817 |
| XM_017012174_ | UC | | CAATGGTTTCTTAATATTTA | |
| 5785 | | | | |
| INHBA_ | AUGGCCAUGUUCAACUUU | 914 | TTAAGGCAAGAGTAGATGGCCATGT | 818 |
| XM_017012174_ | AC | | TCAACTTTACAAAATTTCTT | |
| 4169 | | | | |
| INHBA_ | ACCCAUAUUAUCAUAUUC | 915 | TTGTATTAAGCACGGACCCATATTA | 819 |
| XM_017012174_ | AC | | TCATATTCACTTAAAAAAAT | |
| 5169 | | | | |
| INHBA_ | AAUAAUUGGGAACAAUUA | 916 | TGGAAAAAAGTTTCTAATAATTGGG | 820 |
| XM_017012174_ | CA | | AACAATTACATTAATTAATA | |
| 3792 | | | | |
| INHBA_ | UUAGCCCUAUGUCAAUUU | 917 | AAAATATTGAAGCTTTTAGCCCTAT | 821 |
| XM_017012174_ | GU | | GTCAATTTGTAGATTAAAAT | |
| 3843 | | | | |
| INHBA_ | UGUUGGAUCCAAACAUUU | 918 | ATATGTTTGTCCATCTGTTGGATCC | 822 |
| XM_017012174_ | CU | | AAACATTTCTATATTTTGTA | |
| 2395 | | | | |
| INHBA_ | AAAGAGAUAACAGUUUGA | 919 | ATTTACAATATGTTTAAAGAGATAA | 823 |
| XM_017012174_ | UA | | CAGTTTGATATGTTTTCATG | |
| 5857 | | | | |
| INHBA_ | GGACCCAUAUUAUCAUAU | 920 | AGTTGTATTAAGCACGGACCCATAT | 824 |
| XM_017012174_ | UC | | TATCATATTCACTTAAAAAA | |
| 5167 | | | | |
| INHBA_ | AUGGGAUUCUUAGUAGUA | 921 | TTTAAATTATAACATATGGGATTCT | 825 |
| XM_017012174_ | UG | | TAGTAGTATGTTTTTTTCTT | |
| 4722 | | | | |
| INHBA_ | UAGCCCUAUGUCAAUUUG | 922 | AAATATTGAAGCTTTTAGCCCTATG | 826 |
| XM_017012174_ | UA | | TCAATTTGTAGATTAAAATA | |
| 3844 | | | | |
| INHBA_ | UGGGUUAUCAAACACCCA | 923 | TGGATATTGAGTGATTGGGTTATCA | 827 |
| XM_017012174_ | CA | | AACACCCACAAACTTTAATT | |
| 4519 | | | | |
| INHBA_ | GGGAACAAUUACAUUAAU | 924 | AAGTTTCTAATAATTGGGAACAATT | 828 |
| XM_017012174_ | UA | | ACATTAATTAATATTTTGTA | |
| 3799 | | | | |
| INHBA_ | UCUAAUAAUUGGGAACAA | 925 | AAATGGAAAAAAGTTTCTAATAATT | 829 |
| XM_017012174_ | UU | | GGGAACAATTACATTAATTA | |
| 3789 | | | | |
| INHBA_ | AAAGGUAGAACAAAUACU | 926 | CCCTGCAGTCTCCTTAAAGGTAGAA | 830 |
| XM_017012174_ | UU | | CAAATACTTTCTATTTTTTT | |
| 5059 | | | | |
| INHBA_ | AUAGCAGAAGUUAUUUAU | 927 | TGTTTTCATGTGTTTATAGCAGAAG | 831 |
| XM_017012174_ | UU | | TTATTTATTTCTATGGCATT | |
| 5892 | | | | |
| INHBA_ | UUAAGCACUUCAGGCUGU | 928 | ATGTCCATACACATTTTAAGCACTT | 832 |
| XM_017012174_ | CA | | CAGGCTGTCATTTTTTAATG | |
| 2977 | | | | |
| INHBA_ | UAUAACAUAUGGGAUUCU | 929 | GTAAAGGCTTTAAATTATAACATAT | 833 |
| XM_017012174_ | UA | | GGGATTCTTAGTAGTATGTT | |
| 4714 | | | | |
| INHBA_ | UAUGCAACCUAAUAGGUU | 930 | TTATGATCTTAGAACTATGCAACCT | 834 |
| XM_017012174_ | UG | | AATAGGTTTGAAACTGTTTA | |
| 2262 | | | | |
| INHBA_ | AACCACAUGUCUCAGCUA | 931 | TCAAGAGCAGAAGCAAACCACATGT | 835 |
| XM_017012174_ | UA | | CTCAGCTATATTATTATTTA | |
| 5419 | | | | |
| INHBA_ | UAUGGAAAGAAUGAAAGA | 932 | AAAATTGAGAACAAGTATGGAAAGA | 836 |
| XM_017012174_ | UC | | ATGAAAGATCAAGGAAAAAA | |
| 2188 | | | | |
| INHBA_ | GAGGGCAGAAAUGAAUGA | 933 | GGATGACATTGGAAGGAGGGCAGAA | 837 |
| XM_017012174_ | AC | | ATGAATGAACTTATGGAGCA | |
| 556 | | | | |
| INHBA_ | AGACCCAUGUCCAUGUUG | 934 | GTGCCCACCAAGCTGAGACCCATGT | 838 |
| XM_017012174_ | UA | | CCATGTTGTACTATGATGAT | |
| 1418 | | | | |
| INHBA_ | UUUAAUAGUAACAUGUAU | 935 | TGTCTTTAAATATCTTTTAATAGTA | 839 |
| XM_017012174_ | UU | | ACATGTATTTTATGGACCAA | |
| 4878 | | | | |
| INHBA_ | AUGGCUUUGAAAUAGAAG | 936 | TTTTGTTAAATTTATATGGCTTTGA | 840 |
| XM_017012174_ | UA | | AATAGAAGTATAAGTTGCTA | |
| 4562 | | | | |
| INHBA_ | UUUAAAGAGAUAACAGUU | 937 | TTTATTTACAATATGTTTAAAGAGA | 841 |
| XM_017012174_ | UG | | TAACAGTTTGATATGTTTTC | |
| 5854 | | | | |
| INHBA_ | UUGACAAUGGGUUGAUAU | 938 | TTGAAAAACAGTGCATTGACAATGG | 842 |
| XM_017012174_ | UU | | GTTGATATTTTTCTTTAAAA | |
| 5535 | | | | |
| INHBA_ | ACAGUAGUUGUACACUGU | 939 | TTTCATACTGGATGTACAGTAGTTG | 843 |
| XM_017012174_ | UG | | TACACTGTTGGATATCAAGT | |
| 2839 | | | | |
| INHBA_ | GUGUUUAUAGCAGAAGUU | 940 | TTGATATGTTTTCATGTGTTTATAG | 844 |
| XM_017012174_ | AU | | CAGAAGTTATTTATTTCTAT | |
| 5886 | | | | |
| INHBA_ | UAAGGCAAGAGUAGAUGG | 941 | TGGTGAGAAAAGTTTTAAGGCAAGA | 845 |
| XM_017012174_ | CC | | GTAGATGGCCATGTTCAACT | |
| 4155 | | | | |
| INHBA_ | UUGUGCAUAUGCAUAUCU | 942 | GTATATATACATTGCTTGTGCATAT | 846 |
| XM_017012174_ | GU | | GCATATCTGTATGTATATAT | |
| 2918 | | | | |
| INHBA_ | AAGAUAAUCUGAAGCCUG | 943 | ATAATTATGAAAGCCAAGATAATCT | 847 |
| XM_017012174_ | UU | | GAAGCCTGTTTTATTTTAAA | |
| 5587 | | | | |
| INHBA_ | UUUAAGGUUUCUGAGUUA | 944 | CAAAATGAAGAAATTTTTAAGGTTT | 848 |
| XM_017012174_ | AC | | CTGAGTTAACCAGAAAAATA | |
| 1579 | | | | |
| INHBA_ | GUUGGAUCCAAACAUUUC | 945 | TATGTTTGTCCATCTGTTGGATCCA | 849 |
| XM_017012174_ | UA | | AACATTTCTATATTTTGTAA | |
| 2396 | | | | |
| INHBA_ | AGAAGCAGAGAAAUGUAA | 946 | GTGACCACAGTTATCAGAAGCAGAG | 850 |
| XM_017012174_ | UU | | AAATGTAATTCCATATTTTA | |
| 4449 | | | | |
| INHBA_ | UUUGUAUUGGAAGUAAUC | 947 | AAAAAGAGAGACTTTTTTGTATTGG | 851 |
| XM_017012174_ | UG | | AAGTAATCTGATTAATTTTT | |
| 2320 | | | | |
| INHBA_ | GUGCAUAUGCAUAUCUGU | 948 | ATATATACATTGCTTGTGCATATGC | 852 |
| XM_017012174_ | AU | | ATATCTGTATGTATATATAC | |
| 2920 | | | | |
| INHBA_ | UUGUAUUGGAAGUAAUCU | 949 | AAAAGAGAGACTTTTTTGTATTGGA | 853 |
| XM_017012174_ | GA | | AGTAATCTGATTAATTTTTA | |
| 2321 | | | | |
| INHBA_ | ACUGGGCAAAUACACUUA | 950 | GTAGGCAATGTCACTACTGGGCAAA | 854 |
| XM_017012174_ | CU | | TACACTTACTGTGTTCTAGA | |
| 3999 | | | | |
| INHBA_ | ACACCAAUCUUAGAAGCU | 951 | CTTAAAAATCTGCCCACACCAATCT | 855 |
| XM_017012174_ | CU | | TAGAAGCTCTGAAAAGAATT | |
| 4833 | | | | |
| INHBA_ | UUCAACUAUGUCAAUGGU | 952 | TTTATATGAGTGCATTTCAACTATG | 856 |
| XM_017012174_ | UU | | TCAATGGTTTCTTAATATTT | |
| 5784 | | | | |
| INHBA_ | AUGGUUUCUUAAUAUUUA | 953 | ATTTCAACTATGTCAATGGTTTCTT | 857 |
| XM_017012174_ | UU | | AATATTTATTGTGTAGAAGT | |
| 5797 | | | | |
| INHBA_ | CCAACCAAUUUCAUAUUU | 954 | TCTTATCCCAGAAGACCAACCAATT | 858 |
| XM_017012174_ | AU | | TCATATTTATTTAAGATTGA | |
| 4988 | | | | |
| INHBA_ | UUAACCAAUGUCUGUUUG | 955 | ACTACATGCAGTTCTTTAACCAATG | 859 |
| XM_017012174_ | GC | | TCTGTTTGGCTAATGTAATT | |
| 5728 | | | | |
| INHBA_ | AAUCUUAAAUCAGAAGCU | 956 | GTGTCTATTGGAATCAATCTTAAAT | 860 |
| XM_017012174_ | UU | | CAGAAGCTTTTTCAGAAAAA | |
| 3297 | | | | |
| INHBA_ | UGUAUCACUGAGAAGCUG | 957 | GTTTAAATTAAACCCTGTATCACTG | 861 |
| XM_017012174_ | UU | | AGAAGCTGTTGTATGGGTCA | |
| 5341 | | | | |
| INHBA_ | UUAAGAAGGAUAGGCUAU | 958 | CAAAACACAGTATTTTTAAGAAGGA | 862 |
| XM_017012174_ | AG | | TAGGCTATAGTTTTTGCTTT | |
| 3048 | | | | |
| INHBA_ | AAUACCAAGUUACAUUUC | 959 | GATCAAGGAAAAAAGAATACCAAGT | 863 |
| XM_017012174_ | GU | | TACATTTCGTTAAGGTGCTT | |
| 2219 | | | | |
| INHBA_ | UAUAGCAGAAGUUAUUUA | 960 | ATGTTTTCATGTGTTTATAGCAGAA | 864 |
| XM_017012174_ | UU | | GTTATTTATTTCTATGGCAT | |
| 5891 | | | | |
| Inhba_NM_ | UCAACUAUGUCAAUGGUU | 961 | TTATATGAGTGCATTTCAACTATGT | 865 |
| 008380_6118 | UC | | CAATGGTTTCTTAATATTTA | |
| Inhba_NM_ | UUUAACAAUAUUAGAUAU | 962 | TTAAATTGCTAATATTTTAACAATA | 866 |
| 008380_5037 | UG | | TTAGATATTGTTAACAATTT | |
| Inhba_NM_ | GUUGGAUUCAAACAUUUC | 963 | TATGTTTGTCCATCTGTTGGATTCA | 867 |
| 008380_2941 | UA | | AACATTTCTATATTTTGTAA | |
| Inhba_NM_ | UGUUGGAUUCAAACAUUU | 964 | ATATGTTTGTCCATCTGTTGGATTC | 868 |
| 008380_2940 | CU | | AAACATTTCTATATTTTGTA | |
| Inhba_NM_ | CUAAUCAGUUAUUCAUUU | 965 | GCTTGTCTAAAACAGCTAATCAGTT | 869 |
| 008380_4144 | AC | | ATTCATTTACATATTTAAAA | |
| Inhba_NM_ | UGCAAUAGAAUUUAAAUU | 966 | AAATTTGTATGGCCTTGCAATAGAA | 870 |
| 008380_5011 | GC | | TTTAAATTGCTAATATTTTA | |
| Inhba_NM_ | UAUUAUAAUUAUAAUUAU | 967 | AATAGCTATTTTTATTATTATAATT | 871 |
| 008380_3138 | UA | | ATAATTATTATTATTATTTT | |
| Inhba_NM_ | UAUGCAACCUAAUAGGUU | 968 | TTTTGATCCTAGAACTATGCAACCT | 872 |
| 008380_2807 | UG | | AATAGGTTTGAAACTGTTTA | |
| Inhba_NM_ | UUAAGCACUUCAGGCUGU | 969 | ATGTCCATACACATTTTAAGCACTT | 873 |
| 008380_3507 | CA | | CAGGCTGTCATTTTAAAAAT | |
| Inhba_NM_ | AGCGCCCUCUGAACUGUU | 970 | TTGTGAAAAACCTTTAGCGCCCTCT | 874 |
| 008380_4669 | UC | | GAACTGTTTCTTTAGTATTG | |
| Inhba_NM_ | UUAACAAUAUUAGAUAUU | 971 | TAAATTGCTAATATTTTAACAATAT | 875 |
| 008380_5038 | GU | | TAGATATTGTTAACAATTTA | |
| Inhba_NM_ | UUAAAGAGAUAACGGUUG | 972 | TTATTTACGATATGTTTAAAGAGAT | 876 |
| 008380_6187 | GA | | AACGGTTGGATATGTTTTCA | |
| Inhba_NM_0 | UUUAGCAUUAAGUCAGUC | 973 | TAAAATATTGAAGCTTTTAGCATTA | 877 |
| 08380_4287 | CA | | AGTCAGTCCATAGATTAAAG | |
| Inhba_NM_0 | UCAGUUAUUCAUUUACAU | 974 | GTCTAAAACAGCTAATCAGTTATTC | 878 |
| 08380_4148 | AU | | ATTTACATATTTAAAATATA | |
| Inhba_NM_ | GUCCAUAGAUUAAAGUCA | 975 | TTTAGCATTAAGTCAGTCCATAGAT | 879 |
| 008380_4302 | UU | | TAAAGTCATTGTAGGAAAAT | |
| Inhba_NM_ | CAUAAAGUGAAUCAUUUC | 976 | TTATTTATTTTTATGCATAAAGTGA | 880 |
| 008380_5824 | UU | | ATCATTTCTTCTGTATTAAT | |
| Inhba_NM_ | GGCCUUGCAAUAGAAUUU | 977 | TCATCAAATTTGTATGGCCTTGCAA | 881 |
| 008380_5006 | AA | | TAGAATTTAAATTGCTAATA | |
| Inhba_NM_ | GGGAGUUGUACAAAUCUC | 978 | AGCACTCAGGAGCCAGGGAGTTGTA | 882 |
| 008380_3981 | UA | | CAAATCTCTAATATTCTATT | |
| Inhba_NM_ | AUUGACAAUGGUUGAUAU | 979 | TTTGAAAAACAGTGCATTGACAATG | 883 |
| 008380_5900 | UU | | GTTGATATTTTTCTTTAAAA | |
| Inhba_NM_ | UAUAGCAGAAGUUAUUUA | 980 | ATGTTTTCATGTGTTTATAGCAGAA | 884 |
| 008380_6223 | UU | | GTTATTTATTTCTATTCCAT | |
| Inhba_NM_ | ACCGUCAAUAUACCUUAU | 981 | ATATGTATTAAATATACCGTCAATA | 885 |
| 008380_4413 | CA | | TACCTTATCACATTAGGCTG | |
| Inhba_NM_ | ACGGACUUGUUUGAAGUU | 982 | TAGAGCCTTATTTTTACGGACTTGT | 886 |
| 008380_4191 | UG | | TTGAAGTTTGAAAAACTTTA | |
| Inhba_NM_ | GGCUAAUGUAAUUAAAGU | 983 | CTTTAACCAATGTCTGGCTAATGTA | 887 |
| 008380_6078 | UG | | ATTAAAGTTGTTAATTTATA | |
| Inhba_NM_ | UCAGCUAUAUUAUUAUUU | 984 | AGCAAATCACGTGTCTCAGCTATAT | 888 |
| 008380_5797 | AU | | TATTATTTATTTTTATGCAT | |
| Inhba_NM_ | UGCAUAAGAAAUGAAUUA | 985 | AGGAAATGCATGTACTGCATAAGAA | 889 |
| 008380_4535 | UA | | ATGAATTATAATTAAGGTCT | |
| Inhba_NM_ | UACAGUAGUGUACACUGU | 986 | ATTTCATACTGGATGTACAGTAGTG | 890 |
| 008380_3372 | UG | | TACACTGTTGGATTATCAAG | |
| Inhba_NM_ | UAUAAUUAUAAUUAUUAU | 987 | AGCTATTTTTATTATTATAATTATA | 891 |
| 008380_3141 | UA | | ATTATTATTATTATTTTGTA | |
| Inhba_NM_ | UUGUACAAAUCUCUAAUA | 988 | TCAGGAGCCAGGGAGTTGTACAAAT | 892 |
| 008380_3986 | UU | | CTCTAATATTCTATTTTGCA | |
| Inhba_NM_ | GUGCAUAUGCAUAUCUGU | 989 | ATATATACATTGCTTGTGCATATGC | 893 |
| 008380_3450 | AU | | ATATCTGTATGTATATATAC | |
| Inhba_NM_ | UUCAACUUAUAUUUAUUA | 990 | AAATGGGCTGATTTTTTCAACTTAT | 894 |
| 008380_3810 | UU | | ATTTATTATTTATTGGAATC | |
| Inhba_NM_ | UCAGCAAUGCCCGAUAGC | 991 | GTTAGTAGGCAGTATTCAGCAATGC | 895 |
| 008380_6318 | UU | | CCGATAGCTTCTTTGGCCTT | |
| Inhba_NM_ | UUCAACUAUGUCAAUGGU | 992 | TTTATATGAGTGCATTTCAACTATG | 896 |
| 008380_6117 | UU | | TCAATGGTTTCTTAATATTT | |
| Inhba_NM_ | AUGGUUUCUUAAUAUUUA | 993 | ATTTCAACTATGTCAATGGTTTCTT | 897 |
| 008380_6130 | UU | | AATATTTATTTTGTAGAAGT | |
| Inhba_NM_ | AUGGGAAGAGCAGGAUUU | 994 | GAGATGATCTAGACCATGGGAAGAG | 898 |
| 008380_4771 | AU | | CAGGATTTATGTAACTACTA | |
| Inhba_NM_ | UUUGCCAACCUUAAGUCA | 995 | AGGGGTCACAGCCCCTTTGCCAACC | 899 |
| 008380_1767 | UG | | TTAAGTCATGCTGTGTGCCC | |
| Inhba_NM_ | AGUGCAUUGACAAUGGUU | 996 | AATGCTTTGAAAAACAGTGCATTGA | 900 |
| 008380_5895 | GA | | CAATGGTTGATATTTTTCTT | |
| Inhba_NM_ | GCUAAUGUAAUUAAAGUU | 997 | TTTAACCAATGTCTGGCTAATGTAA | 901 |
| 008380_6079 | GU | | TTAAAGTTGTTAATTTATAT | |
| Inhba_NM_ | UUUAAGAAGGAUAAGUGA | 998 | GCAAAACACAGTATTTTTAAGAAGG | 902 |
| 008380_3578 | UA | | ATAAGTGATAGATTTTTTTT | |
| Inhba_NM_ | GUCUAUGUGACACUAUCU | 999 | CCAGGTTATAAGTATGTCTATGTGA | 903 |
| 008380_2405 | CU | | CACTATCTCTGTGTATTTCA | |
| Inhba_NM_ | AAUACCAAGUUACAUUUC | 1000 | GACCAAGGGAAGAAGAATACCAAGT | 904 |
| 008380_2764 | GU | | TACATTTCGTTAAGGTGCTT | |
| Inhba_NM_ | CUUGAAAGGAAUAUGUUU | 1001 | CTTCAAGGAGAGATACTTGAAAGGA | 905 |
| 008380_2915 | GU | | ATATGTTTGTCCATCTGTTG | |
| Inhba_NM_ | GUGUUUAUAGCAGAAGUU | 1002 | TGGATATGTTTTCATGTGTTTATAG | 906 |
| 008380_6218 | AU | | CAGAAGTTATTTATTTCTAT | |
| Inhba_NM_ | AUGGCCUUGCAAUAGAAU | 1003 | ACTCATCAAATTTGTATGGCCTTGC | 907 |
| 008380_5004 | UU | | AATAGAATTTAAATTGCTAA | |
| Inhba_NM_ | UUUGACCUGUCUAAUCCU | 1004 | TGCACTACGATGGTGTTTGACCTGT | 908 |
| 008380_3013 | UA | | CTAATCCTTATTTAACAAGT | |
| Inhba_NM_ | UAACCAAUGUCUGGCUAA | 1005 | CTACATGCAATTCTTTAACCAATGT | 909 |
| 008380_6066 | UG | | CTGGCTAATGTAATTAAAGT | |
| Inhba_NM_ | ACAAACAUGCCUCAGUUA | 1005 | CTTTGTGATTCCAGAACAAACATGC | 910 |
| 008380_5511 | UA | | CTCAGTTATAGTAACATGCA | |
| Inhba_NM_ | AGAUAUUGUUAACAAUUU | 1006 | TATTTTAACAATATTAGATATTGTT | 911 |
| 008380_5049 | AG | | AACAATTTAGAAAACATGAA | |
| Inhba_NM_ | UUUGUAGAAGUGCUGGUA | 1007 | TTTCTTAATATTTATTTTGTAGAAG | 912 |
| 008380_6149 | AU | | TGCTGGTAATTTTTTATTTA | |

In some embodiments, a target region of an INHBA specific siRNA comprises the nucleic acid sequence of any one of SEQ ID NOs: 817-912.

In some embodiments, an INHBA specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides to an INHBA target nucleic acid sequence as set forth in SEQ ID NOs: 817-912. In some embodiments, an INHBA specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides of to an INHBA target nucleic acid sequence as set forth in SEQ ID NOs: 817-912, except for at least 1, at least 2, at least 3, at least 4, or at least 5 mismatch within the region of complementarity to the nucleotide sequence of SEQ ID NOs: 817-912.

In some embodiments, an INHBA specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides to an INHBA target nucleic acid sequence as set forth in SEQ ID NOs: 849, 851, or 853. In some embodiments, an INHBA specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides of to an INHBA target nucleic acid sequence as set forth in SEQ ID NO: 849, 851, or 853, except for at least 1, at least 2, at least 3, at least 4, or at least 5 mismatch within the region of complementarity to the nucleotide sequence of SEQ ID NO: 849, 851, or 853.

In some embodiments, an INHBA specific siRNA may be at least 80%, at least 85%, at least 90%, at least 91%, at least 92%, at least 93%, at least 94%, at least 95%, at least 96%, at least 97%, at least 98%, at least 99% or 100% complementary to any one of SEQ ID NOs: 913-1007. In some embodiments, a complementary nucleotide sequence need not be 100% complementary to that of INHBA target sequence to be specifically hybridizable or specific for an INHBA. In some embodiments, the antisense strand of an INHBA specific siRNA is fully complementarity with all or a portion of its target sequence. In some embodiments, the antisense strand of an INHBA specific siRNA includes 1, 2, 3, or more mismatches to the target sequence. In some embodiments, an INHBA specific siRNA comprises an antisense strand comprising a nucleotide sequence that is complementary (e.g., at least 85%, at least 90%, at least 95%, at least 96%, at least 97%, at least 98%, at least 99%, or 100%) to a target RNA sequence as set forth in any one of SEQ ID NOs: 913-1007. In some embodiments, an INHBA specific siRNA comprises an antisense strand comprising a nucleic acid sequence that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in any one of SEQ ID NOs: 913-1007. In some embodiments, an INHBA specific siRNA comprises an antisense strand comprising a nucleic acid sequence that is complementary the sequence as set forth in any one of SEQ ID NOs: 913-1007.

In some embodiments, an INHBA specific siRNA may be at least 80%, at least 85%, at least 90%, at least 91%, at least 92%, at least 93%, at least 94%, at least 95%, at least 96%, at least 97%, at least 98%, at least 99% or 100% complementary to SEQ ID NO: 945, 947, or 949.

In some embodiments, a complementary nucleotide sequence need not be 100% complementary to that of INHBA target sequence to be specifically hybridizable or specific for an INHBA. In some embodiments, the antisense strand of an INHBA specific siRNA is fully complementarity with all or a portion of its target sequence. In some embodiments, the antisense strand of an INHBA specific siRNA includes 1, 2, 3, or more mismatches to the target sequence. In some embodiments, an INHBA specific siRNA comprises an antisense strand comprising a nucleotide sequence that is complementary (e.g., at least 85%, at least 90%, at least 95%, at least 96%, at least 97%, at least 98%, at least 99%, or 100%) to a target RNA sequence as set forth in any one of SEQ ID NO: 945, 947, or 949. In some embodiments, an INHBA specific siRNA comprises an antisense strand comprising a nucleic acid sequence that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in any one of SEQ ID NO: 945. In some embodiments, an INHBA specific siRNA comprises an antisense strand comprising a nucleic acid sequence that is complementary the sequence as set forth in any one of SEQ ID NO: 945, 947, 949.

Exemplary INHBA specific siRNA sequences (both antisense and sense strand) are set forth in Table 10.

TABLE 10
Exemplary human INHBA or mouse Inhba specific siRNA sequences without
mismatch

| NCBI Accession | | SEQ ID | | SEQ ID |
|---|---|---|---|---|
| No and position | Sense Strand | NO | Antisense Strand | NO |
| INHBA_XM_ | CUAUGUCAAUGGUUUA | 1104 | UAAACCAUUGACAUAGUUGUU | 1008 |
| 017012174_5785 | | | | |
| INHBA_XM_ | CCAUGUUCAACUUUAA | 1105 | UUAAAGUUGAACAUGGCCAUU | 1009 |
| 017012174_4169 | | | | |
| INHBA_XM_ | AUAUUAUCAUAUUCAA | 1106 | UUGAAUAUGAUAAUAUGGGUU | 1010 |
| 017012174_5169 | | | | |
| INHBA_XM_ | AUUGGGAACAAUUACA | 1107 | UGUAAUUGUUCCCAAUUAUUU | 1011 |
| 017012174_3792 | | | | |
| INHBA_XM_ | CCCUAUGUCAAUUUGA | 1108 | UCAAAUUGACAUAGGGCUAUU | 1012 |
| 017012174_3843 | | | | |
| INHBA_XM_ | GGAUCCAAACAUUUCA | 1109 | UGAAAUGUUUGGAUCCAACUU | 1013 |
| 017012174_2395 | | | | |
| INHBA_XM_ | AGAUAACAGUUUGAUA | 1110 | UAUCAAACUGUUAUCUCUUUU | 1014 |
| 017012174_5857 | | | | |
| INHBA_XM_ | CCAUAUUAUCAUAUUA | 1111 | UAAUAUGAUAAUAUGGGUCUU | 1015 |
| 017012174_5167 | | | | |
| INHBA_XM_ | GAUUCUUAGUAGUAUA | 1112 | UAUACUACUAAGAAUCCCAUU | 1016 |
| 017012174_4722 | | | | |
| INHBA_XM_ | CCUAUGUCAAUUUGUA | 1113 | UACAAAUUGACAUAGGGCUUU | 1017 |
| 017012174_3844 | | | | |
| INHBA_XM_ | UUAUCAAACACCCACA | 1114 | UGUGGGUGUUUGAUAACCCUU | 1018 |
| 017012174_4519 | | | | |
| INHBA_XM_ | ACAAUUACAUUAAUUA | 1115 | UAAUUAAUGUAAUUGUUCCUU | 1019 |
| 017012174_3799 | | | | |
| INHBA_XM_ | AUAAUUGGGAACAAUA | 1116 | UAUUGUUCCCAAUUAUUAGUU | 1020 |
| 017012174_3789 | | | | |
| INHBA_XM_ | GUAGAACAAAUACUUA | 1117 | UAAGUAUUUGUUCUACCUUUU | 1021 |
| 017012174_5059 | | | | |
| INHBA_XM_ | CAGAAGUUAUUUAUUA | 1118 | UAAUAAAUAACUUCUGCUAUU | 1022 |
| 017012174_5892 | | | | |
| INHBA_XM_ | GCACUUCAGGCUGUCA | 1119 | UGACAGCCUGAAGUGCUUAUU | 1023 |
| 017012174_2977 | | | | |
| INHBA_XM_ | ACAUAUGGGAUUCUUA | 1120 | UAAGAAUCCCAUAUGUUAUUU | 1024 |
| 017012174_4714 | | | | |
| INHBA_XM_ | CAACCUAAUAGGUUUA | 1121 | UAAACCUAUUAGGUUGCAUUU | 1025 |
| 017012174_2262 | | | | |
| INHBA_XM_ | ACAUGUCUCAGCUAUA | 1122 | UAUAGCUGAGACAUGUGGUUU | 1026 |
| 017012174_5419 | | | | |
| INHBA_XM_ | GAAAGAAUGAAAGAUA | 1123 | UAUCUUUCAUUCUUUCCAUUU | 1027 |
| 017012174_2188 | | | | |
| INHBA_XM_ | GCAGAAAUGAAUGAAA | 1124 | UUUCAUUCAUUUCUGCCCUUU | 1028 |
| 017012174_556 | | | | |
| INHBA_XM_ | CCAUGUCCAUGUUGUA | 1125 | UACAACAUGGACAUGGGUCUU | 1029 |
| 017012174_1418 | | | | |
| INHBA_XM_ | AUAGUAACAUGUAUUA | 1126 | UAAUACAUGUUACUAUUAAUU | 1030 |
| 017012174_4878 | | | | |
| INHBA_XM_ | CUUUGAAAUAGAAGUA | 1127 | UACUUCUAUUUCAAAGCCAUU | 1031 |
| 017012174_4562 | | | | |
| INHBA_XM_ | AAGAGAUAACAGUUUA | 1128 | UAAACUGUUAUCUCUUUAAUU | 1032 |
| 017012174_5854 | | | | |
| INHBA_XM_ | CAAUGGGUUGAUAUUA | 1129 | UAAUAUCAACCCAUUGUCAUU | 1033 |
| 017012174_5535 | | | | |
| INHBA_XM_ | UAGUUGUACACUGUUA | 113 | UAACAGUGUACAACUACUGUU | 1034 |
| 017012174_2839 | | 0 | | |
| INHBA_XM_ | UUAUAGCAGAAGUUAA | 1131 | UUAACUUCUGCUAUAAACAUU | 1035 |
| 017012174_5886 | | | | |
| INHBA_XM_ | GCAAGAGUAGAUGGCA | 1132 | UGCCAUCUACUCUUGCCUUUU | 1036 |
| 017012174_4155 | | | | |
| INHBA_XM_ | GCAUAUGCAUAUCUGA | 1133 | UCAGAUAUGCAUAUGCACAUU | 1037 |
| 017012174_2918 | | | | |
| INHBA_XM_ | UAAUCUGAAGCCUGUA | 1134 | UACAGGCUUCAGAUUAUCUUU | 1038 |
| 017012174_5587 | | | | |
| INHBA_XM_ | AGGUUUCUGAGUUAAA | 1135 | UUUAACUCAGAAACCUUAAUU | 1039 |
| 017012174_1579 | | | | |
| INHBA_XM_ | GAUCCAAACAUUUCUA | 1136 | UAGAAAUGUUUGGAUCCAAUU | 1040 |
| 017012174_2396 | | | | |
| INHBA_XM_ | GCAGAGAAAUGUAAUA | 1137 | UAUUACAUUUCUCUGCUUCUU | 1041 |
| 017012174_4449 | | | | |
| INHBA_XM_ | UAUUGGAAGUAAUCUA | 1138 | UAGAUUACUUCCAAUACAAUU | 1042 |
| 017012174_2320 | | | | |
| INHBA_XM_ | AUAUGCAUAUCUGUAA | 1139 | UUACAGAUAUGCAUAUGCAUU | 1043 |
| 017012174_2920 | | | | |
| INHBA_XM_ | AUUGGAAGUAAUCUGA | 1140 | UCAGAUUACUUCCAAUACAUU | 1044 |
| 017012174_2321 | | | | |
| INHBA_XM_ | GGCAAAUACACUUACA | 1141 | UGUAAGUGUAUUUGCCCAGUU | 1045 |
| 017012174_3999 | | | | |
| INHBA_XM_ | CAAUCUUAGAAGCUCA | 1142 | UGAGCUUCUAAGAUUGGUGUU | 1046 |
| 017012174_4833 | | | | |
| INHBA_XM_ | ACUAUGUCAAUGGUUA | 1143 | UAACCAUUGACAUAGUUGAUU | 1047 |
| 017012174_5784 | | | | |
| INHBA_XM_ | UUUCUUAAUAUUUAUA | 1144 | UAUAAAUAUUAAGAAACCAUU | 1048 |
| 017012174_5797 | | | | |
| INHBA_XM_ | CCAAUUUCAUAUUUAA | 1145 | UUAAAUAUGAAAUUGGUUGUU | 1049 |
| 017012174_4988 | | | | |
| INHBA_XM_ | CCAAUGUCUGUUUGGA | 1146 | UCCAAACAGACAUUGGUUAUU | 1050 |
| 017012174_5728 | | | | |
| INHBA_XM_ | UUAAAUCAGAAGCUUA | 1147 | UAAGCUUCUGAUUUAAGAUUU | 1051 |
| 017012174_3297 | | | | |
| INHBA_XM_ | UCACUGAGAAGCUGUA | 1148 | UACAGCUUCUCAGUGAUACUU | 1052 |
| 017012174_5341 | | | | |
| INHBA_XM_ | GAAGGAUAGGCUAUAA | 1149 | UUAUAGCCUAUCCUUCUUAUU | 1053 |
| 017012174_3048 | | | | |
| INHBA_XM_ | CCAAGUUACAUUUCGA | 1150 | UCGAAAUGUAACUUGGUAUUU | 1054 |
| 017012174_2219 | | | | |
| INHBA_XM_ | GCAGAAGUUAUUUAUA | 1151 | UAUAAAUAACUUCUGCUAUUU | 1055 |
| 017012174_5891 | | | | |
| Inhba_NM_ | CUAUGUCAAUGGUUUA | 1152 | UAAACCAUUGACAUAGUUGUU | 1056 |
| 0083806118 | | | | |
| Inhba_NM_008380_ | ACAAUAUUAGAUAUUA | 1153 | UAAUAUCUAAUAUUGUUAAUU | 1057 |
| 5037 | | | | |
| Inhba_NM_008380_ | GAUUCAAACAUUUCUA | 1154 | UAGAAAUGUUUGAAUCCAAUU | 1058 |
| 2941 | | | | |
| Inhba_NM_008380_ | GGAUUCAAACAUUUCA | 1155 | UGAAAUGUUUGAAUCCAACUU | 1059 |
| 2940 | | | | |
| Inhba_NM_008380_ | UCAGUUAUUCAUUUAA | 1156 | UUAAAUGAAUAACUGAUUAUU | 1060 |
| 4144 | | | | |
| Inhba_NM_008380_ | AUAGAAUUUAAAUUGA | 1157 | UCAAUUUAAAUUCUAUUGCUU | 1061 |
| 5011 | | | | |
| Inhba_NM_008380_ | AUAAUUAUAAUUAUUA | 1158 | UAAUAAUUAUAAUUAUAAUUU | 1062 |
| 3138 | | | | |
| Inhba_NM_008380_ | CAACCUAAUAGGUUUA | 1159 | UAAACCUAUUAGGUUGCAUUU | 1063 |
| 2807 | | | | |
| Inhba_NM_008380_ | GCACUUCAGGCUGUCA | 1160 | UGACAGCCUGAAGUGCUUAUU | 1064 |
| 3507 | | | | |
| Inhba_NM_008380_ | CCCUCUGAACUGUUUA | 1161 | UAAACAGUUCAGAGGGCGCUU | 1065 |
| 4669 | | | | |
| Inhba_NM_008380_ | CAAUAUUAGAUAUUGA | 1162 | UCAAUAUCUAAUAUUGUUAUU | 1066 |
| 5038 | | | | |
| Inhba_NM_008380_ | AGAGAUAACGGUUGGA | 1163 | UCCAACCGUUAUCUCUUUAUU | 1067 |
| 6187 | | | | |
| Inhba_NM_008380_ | GCAUUAAGUCAGUCCA | 1164 | UGGACUGACUUAAUGCUAAUU | 1068 |
| 4287 | | | | |
| Inhba_NM_008380_ | UUAUUCAUUUACAUAA | 1165 | UUAUGUAAAUGAAUAACUGUU | 1069 |
| 4148 | | | | |
| Inhba_NM_008380_ | AUAGAUUAAAGUCAUA | 1166 | UAUGACUUUAAUCUAUGGAUU | 1070 |
| 4302 | | | | |
| Inhba_NM_008380_ | AAGUGAAUCAUUUCUA | 1167 | UAGAAAUGAUUCACUUUAUUU | 1071 |
| 5824 | | | | |
| Inhba_NM_008380_ | UUGCAAUAGAAUUUAA | 1168 | UUAAAUUCUAUUGCAAGGCUU | 1072 |
| 5006 | | | | |
| Inhba_NM_008380_ | GUUGUACAAAUCUCUA | 1169 | UAGAGAUUUGUACAACUCCUU | 1073 |
| 3981 | | | | |
| Inhba_NM_008380_ | ACAAUGGUUGAUAUUA | 1170 | UAAUAUCAACCAUUGUCAAUU | 1074 |
| 5900 | | | | |
| Inhba_NM_008380_ | GCAGAAGUUAUUUAUA | 1171 | UAUAAAUAACUUCUGCUAUUU | 1075 |
| 6223 | | | | |
| Inhba_NM_008380_ | UCAAUAUACCUUAUCA | 1172 | UGAUAAGGUAUAUUGACGGUU | 1076 |
| 4413 | | | | |
| Inhba_NM_008380_ | ACUUGUUUGAAGUUUA | 1173 | UAAACUUCAAACAAGUCCGUU | 1077 |
| 4191 | | | | |
| Inhba_NM_008380_ | AAUGUAAUUAAAGUUA | 1174 | UAACUUUAAUUACAUUAGCUU | 1078 |
| 6078 | | | | |
| Inhba_NM_008380_ | CUAUAUUAUUAUUUAA | 1175 | UUAAAUAAUAAUAUAGCUGUU | 1079 |
| 5797 | | | | |
| Inhba_NM_008380_ | UAAGAAAUGAAUUAUA | 1176 | UAUAAUUCAUUUCUUAUGCUU | 1080 |
| 4535 | | | | |
| Inhba_NM_008380_ | GUAGUGUACACUGUUA | 1177 | UAACAGUGUACACUACUGUUU | 1081 |
| 3372 | | | | |
| Inhba_NM_008380_ | AUUAUAAUUAUUAUUA | 1178 | UAAUAAUAAUUAUAAUUAUUU | 1082 |
| 3141 | | | | |
| Inhba_NM_008380_ | ACAAAUCUCUAAUAUA | 1179 | UAUAUUAGAGAUUUGUACAUU | 1083 |
| 3986 | | | | |
| Inhba_NM_008380_ | AUAUGCAUAUCUGUAA | 1180 | UUACAGAUAUGCAUAUGCAUU | 1084 |
| 3450 | | | | |
| Inhba_NM_008380_ | ACUUAUAUUUAUUAUA | 1181 | UAUAAUAAAUAUAAGUUGAUU | 1085 |
| 3810 | | | | |
| Inhba_NM_008380_ | CAAUGCCCGAUAGCUA | 1182 | UAGCUAUCGGGCAUUGCUGUU | 1086 |
| 6318 | | | | |
| Inhba_NM_008380_ | ACUAUGUCAAUGGUUA | 1183 | UAACCAUUGACAUAGUUGAUU | 1087 |
| 6117 | | | | |
| Inhba_NM_008380_ | UUUCUUAAUAUUUAUA | 1184 | UAUAAAUAUUAAGAAACCAUU | 1088 |
| 6130 | | | | |
| Inhba_NM_008380_ | GAAGAGCAGGAUUUAA | 1185 | UUAAAUCCUGCUCUUCCCAUU | 1089 |
| 4771 | | | | |
| Inhba_NM_008380_ | CCAACCUUAAGUCAUA | 1186 | UAUGACUUAAGGUUGGCAAUU | 1090 |
| 1767 | | | | |
| Inhba_NM_008380_ | CAUUGACAAUGGUUGA | 1187 | UCAACCAUUGUCAAUGCACUU | 1091 |
| 5895 | | | | |
| Inhba_NM_008380_ | AUGUAAUUAAAGUUGA | 1188 | UCAACUUUAAUUACAUUAGUU | 1092 |
| 6079 | | | | |
| Inhba_NM_008380_ | AGAAGGAUAAGUGAUA | 1189 | UAUCACUUAUCCUUCUUAAUU | 1093 |
| 3578 | | | | |
| Inhba_NM_008380_ | AUGUGACACUAUCUCA | 1190 | UGAGAUAGUGUCACAUAGAUU | 1094 |
| 2405 | | | | |
| Inhba_NM_008380_ | CCAAGUUACAUUUCGA | 1191 | UCGAAAUGUAACUUGGUAUUU | 1095 |
| 2764 | | | | |
| Inhba_NM_008380_ | AAAGGAAUAUGUUUGA | 1192 | UCAAACAUAUUCCUUUCAAUU | 1096 |
| 2915 | | | | |
| Inhba_NM_008380_ | UUAUAGCAGAAGUUAA | 1193 | UUAACUUCUGCUAUAAACAUU | 1097 |
| 6218 | | | | |
| Inhba_NM_008380_ | CCUUGCAAUAGAAUUA | 1194 | UAAUUCUAUUGCAAGGCCAUU | 1098 |
| 5004 | | | | |
| Inhba_NM_008380_ | ACCUGUCUAAUCCUUA | 1195 | UAAGGAUUAGACAGGUCAAUU | 1099 |
| 3013 | | | | |
| Inhba_NM_008380_ | CAAUGUCUGGCUAAUA | 1196 | UAUUAGCCAGACAUUGGUUUU | 1100 |
| 6066 | | | | |
| Inhba_NM_008380_ | ACAUGCCUCAGUUAUA | 1197 | UAUAACUGAGGCAUGUUUGUU | 1101 |
| 5511 | | | | |
| Inhba_NM_008380_ | AUUGUUAACAAUUUAA | 1198 | UUAAAUUGUUAACAAUAUCUU | 1102 |
| 5049 | | | | |
| Inhba_NM_008380_ | UAGAAGUGCUGGUAAA | 1199 | UUUACCAGCACUUCUACAAUU | 1103 |
| 6149 | | | | |

In some embodiments, an INHBA specific siRNA comprises an antisense strand that comprises at least 15 consecutive nucleosides of (e.g., at least 15, at least 16, at least 17, at least 18, at least 19, or at least 20) the sequence of any one of SEQ ID NOs: 1008-1103. In some embodiments, an INHBA specific siRNA further comprises a sense strand that comprises at least 15 consecutive nucleosides (e.g., at least 15, at least 16, at least 17, at least 18, at least 19, or at least 20) complementary to the sequence of any one of SEQ ID NOs: 1008-1103. In some embodiments, an INHBA specific siRNA further comprises a sense strand that comprises at least 15 consecutive nucleosides of (e.g., at least 15, at least 16, at least 17, at least 18, at least 19, or at least 20) the sequence of any one of SEQ ID NOs: 1104-1199.

In some embodiments, an INHBA specific siRNA comprises an antisense strand that comprises at least 15 consecutive nucleosides of (e.g., at least 15, at least 16, at least 17, at least 18, at least 19, or at least 20) the sequence of SEQ ID NOs: 1040, 1042, or 1044. In some embodiments, an INHBA specific siRNA further comprises a sense strand that comprises at least 15 consecutive nucleosides (e.g., at least 15, at least 16, at least 17, at least 18, at least 19, or at least 20) complementary to the sequence of any one of SEQ ID NOs: 1040, 1042, or 1044. In some embodiments, an INHBA specific siRNA further comprises a sense strand that comprises at least 15 consecutive nucleosides of (e.g., at least 15, at least 16, at least 17, at least 18, at least 19, or at least 20) the sequence of any one of SEQ ID NOs: 1136, 1138, or 1144.

In some embodiments, an INHBA specific siRNA comprises an antisense strand that is at least 80% (e.g., at least 85%, at least 90%, at least 95%, at least 96%, at least 97%, at least 98%, at least 99%, or 100%) to the sequence of any one of SEQ ID NOs: 1008-1103. In some embodiments, an INHBA specific siRNA further comprises a sense strand that comprises a complementary to a sequence at least 80% (e.g., at least 85%, at least 90%, at least 95%, at least 96%, at least 97%, at least 98%, at least 99%, or 100%) identical to the sequence of any one of SEQ ID NOs: 1008-1103. In some embodiments, an INHBA specific siRNA further comprises a sense strand that is at least 80% (e.g., at least 85%, at least 90%, at least 95%, at least 96%, at least 97%, at least 98%, at least 99%, or 100%) identical to the sequence of any one of SEQ ID NOs: 1104-1199.

In some embodiments, an INHBA specific siRNA described herein comprises at least one nucleoside modified at the 2′ position of the sugar. In some embodiments, an INHBA specific siRNA comprises at least one 2′-modified nucleoside. In some embodiments, all of the nucleosides in the oligonucleotide are 2′-modified nucleosides.

In some embodiments, an INHBA specific siRNA described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside. In some embodiments, an INHBA specific siRNA comprises one or more 2′-O-methoxyethyl (2′-MOE) modified nucleoside and/or one or more 2′-fluoro (2′-F) modified nucleoside.

In some embodiments, an INHBA specific siRNA comprises one or more modified one or more modified internucleotide linkages. In some embodiments, the internucleotide linkage is a phosphorothioate linkage. In some embodiments, an INHBA specific siRNA further comprises a cholesterol molecule. In some embodiments, an INHBA specific siRNA is conjugated to the cholesterol by a TEG linker. In some embodiments, an INHBA specific siRNA is conjugated to a docosanoic acid (DCA). In some embodiments, the DCA is conjugated to the isolated nucleic acid via a cleavable linker. Exemplary an INHBA specific siRNA with chemical modifications are set forth in Table 3.

TABLE 3
Modified INHBA specific siRNA without mismatch

| NCBI | | SEQ |
|---|---|---|
| Accession No | | ID |
| and position | | NO |
| | Sense Strand | |
| INHBA_XM_017012174_5785 | (mC)#(mU)#(mA)(fU)(mG)(fU)(mC)(fA)(mA)(fU)(mG)(mG)(mU)(fU)#(mU)#(mA)-TegChol | 93 |
| INHBA_XM_017012174_4169 | (mC)#(mC)#(mA)(fU)(mG)(fU)(mU)(fC)(mA)(fA)(mC)(mU)(mU)(fU)#(mA)#(mA)-TegChol | 94 |
| INHBA_XM_017012174_5169 | (mA)#(mU)#(mA)(fU)(mU)(fA)(mU)(fC)(mA)(fU)(mA)(mU)(mU)(fC)#(mA)#(mA)-TegChol | 95 |
| INHBA_XM_017012174_3792 | (mA)#(mU)#(mU)(fG)(mG)(fG)(mA)(fA)(mC)(fA)(mA)(mU)(mU)(fA)#(mC)#(mA)-TegChol | 96 |
| INHBA_XM_017012174_3843 | (mC)#(mC)#(mC)(fU)(mA)(fU)(mG)(fU)(mC)(fA)(mA)(mU)(mU)(fU)#(mG)#(mA)-TegChol | 97 |
| INHBA_XM_017012174_2395 | (mG)#(mG)#(mA)(fU)(mC)(fC)(mA)(fA)(mA)(fC)(mA)(mU)(mU)(fU)#(mC)#(mA)-TegChol | 98 |
| INHBA_XM_017012174_5857 | (mA)#(mG)#(mA)(fU)(mA)(fA)(mC)(fA)(mG)(fU)(mU)(mU)(mG)(fA)#(mU)#(mA)-TegChol | 99 |
| INHBA_XM_017012174_5167 | (mC)#(mC)#(mA)(fU)(mA)(fU)(mU)(fA)(mU)(fC)(mA)(mU)(mA)(fU)#(mU)#(mA)-TegChol | 100 |
| INHBA_XM_017012174_4722 | (mG)#(mA)#(mU)(fU)(mC)(fU)(mU)(fA)(mG)(fU)(mA)(mG)(mU)(fA)#(mU)#(mA)-TegChol | 101 |
| INHBA_XM_017012174_3844 | (mC)#(mC)#(mU)(fA)(mU)(fG)(mU)(fC)(mA)(fA)(mU)(mU)(mU)(fG)#(mU)#(mA)-TegChol | 102 |
| INHBA_XM_017012174_4519 | (mU)#(mU)#(mA)(fU)(mC)(fA)(mA)(fA)(mC)(fA)(mC)(mC)(mC)(fA)#(mC)#(mA)-TegChol | 103 |
| INHBA_XM_017012174_3799 | (mA)#(mC)#(mA)(fA)(mU)(fU)(mA)(fC)(mA)(fU)(mU)(mA)(mA)(fU)#(mU)#(mA)-TegChol | 104 |
| INHBA_XM_017012174_3789 | (mA)#(mU)#(mA)(fA)(mU)(fU)(mG)(fG)(mG)(fA)(mA)(mC)(mA)(fA)#(mU)#(mA)-TegChol | 105 |
| INHBA_XM_017012174_5059 | (mG)#(mU)#(mA)(fG)(mA)(fA)(mC)(fA)(mA)(fA)(mU)(mA)(mC)(fU)#(mU)#(mA)-TegChol | 106 |
| INHBA_XM_017012174_5892 | (mC)#(mA)#(mG)(fA)(mA)(fG)(mU)(fU)(mA)(fU)(mU)(mU)(mA)(fU)#(mU)#(mA)-TegChol | 107 |
| INHBA_XM_017012174_2977 | (mG)#(mC)#(mA)(fC)(mU)(fU)(mC)(fA)(mG)(fG)(mC)(mU)(mG)(fU)#(mC)#(mA)-TegChol | 108 |
| INHBA_XM_017012174_4714 | (mA)#(mC)#(mA)(fU)(mA)(fU)(mG)(fG)(mG)(fA)(mU)(mU)(mC)(fU)#(mU)#(mA)-TegChol | 109 |
| INHBA_XM_017012174_2262 | (mC)#(mA)#(mA)(fC)(mC)(fU)(mA)(fA)(mU)(fA)(mG)(mG)(mU)(fU)#(mU)#(mA)-TegChol | 110 |
| INHBA_XM_017012174_5419 | (mA)#(mC)#(mA)(fU)(mG)(fU)(mC)(fU)(mC)(fA)(mG)(mC)(mU)(fA)#(mU)#(mA)-TegChol | 111 |
| INHBA_XM_017012174_2188 | (mG)#(mA)#(mA)(fA)(mG)(fA)(mA)(fU)(mG)(fA)(mA)(mA)(mG)(fA)#(mU)#(mA)-TegChol | 112 |
| INHBA_XM_017012174_556 | (mG)#(mC)#(mA)(fG)(mA)(fA)(mA)(fU)(mG)(fA)(mA)(mU)(mG)(fA)#(mA)#(mA)-TegChol | 113 |
| INHBA_XM_017012174_1418 | (mC)#(mC)#(mA)(fU)(mG)(fU)(mC)(fC)(mA)(fU)(mG)(mU)(mU)(fG)#(mU)#(mA)-TegChol | 114 |
| INHBA_XM_017012174_4878 | (mA)#(mU)#(mA)(fG)(mU)(fA)(mA)(fC)(mA)(fU)(mG)(mU)(mA)(fU)#(mU)#(mA)-TegChol | 115 |
| INHBA_XM_017012174_4562 | (mC)#(mU)#(mU)(fU)(mG)(fA)(mA)(fA)(mU)(fA)(mG)(mA)(mA)(fG)#(mU)#(mA)-TegChol | 116 |
| INHBA_XM_017012174_5854 | (mA)#(mA)#(mG)(fA)(mG)(fA)(mU)(fA)(mA)(fC)(mA)(mG)(mU)(fU)#(mU)#(mA)-TegChol | 117 |
| INHBA_XM_017012174_5535 | (mC)#(mA)#(mA)(fU)(mG)(fG)(mG)(fU)(mU)(fG)(mA)(mU)(mA)(fU)#(mU)#(mA)-TegChol | 118 |
| INHBA_XM_017012174_2839 | (mU)#(mA)#(mG)(fU)(mU)(fG)(mU)(fA)(mC)(fA)(mC)(mU)(mG)(fU)#(mU)#(mA)-TegChol | 119 |
| INHBA_XM_017012174_5886 | (mU)#(mU)#(mA)(fU)(mA)(fG)(mC)(fA)(mG)(fA)(mA)(mG)(mU)(fU)#(mA)#(mA)-TegChol | 120 |
| INHBA_XM_017012174_4155 | (mG)#(mC)#(mA)(fA)(mG)(fA)(mG)(fU)(mA)(fG)(mA)(mU)(mG)(fG)#(mC)#(mA)-TegChol | 121 |
| INHBA_XM_017012174_2918 | (mG)#(mC)#(mA)(fU)(mA)(fU)(mG)(fC)(mA)(fU)(mA)(mU)(mC)(fU)#(mG)#(mA)-TegChol | 122 |
| INHBA_XM_017012174_5587 | (mU)#(mA)#(mA)(fU)(mC)(fU)(mG)(fA)(mA)(fG)(mC)(mC)(mU)(fG)#(mU)#(mA)-TegChol | 123 |
| INHBA_XM_017012174_1579 | (mA)#(mG)#(mG)(fU)(mU)(fU)(mC)(fU)(mG)(fA)(mG)(mU)(mU)(fA)#(mA)#(mA)-TegChol | 124 |
| INHBA_XM_017012174_2396 | (mG)#(mA)#(mU)(fC)(mC)(fA)(mA)(fA)(mC)(fA)(mU)(mU)(mU)(fC)#(mU)#(mA)-TegChol | 125 |
| INHBA_XM_017012174_4449 | (mG)#(mC)#(mA)(fG)(mA)(fG)(mA)(fA)(mA)(fU)(mG)(mU)(mA)(fA)#(mU)#(mA)-TegChol | 126 |
| INHBA_XM_017012174_2320 | (mU)#(mA)#(mU)(fU)(mG)(fG)(mA)(fA)(mG)(fU)(mA)(mA)(mU)(fC)#(mU)#(mA)-TegChol | 127 |
| INHBA_XM_017012174_2920 | (mA)#(mU)#(mA)(fU)(mG)(fC)(mA)(fU)(mA)(fU)(mC)(mU)(mG)(fU)#(mA)#(mA)-TegChol | 128 |
| INHBA_XM_017012174_2321 | (mA)#(mU)#(mU)(fG)(mG)(fA)(mA)(fG)(mU)(fA)(mA)(mU)(mC)(fU)#(mG)#(mA)-TegChol | 129 |
| INHBA_XM_017012174_3999 | (mG)#(mG)#(mC)(fA)(mA)(fA)(mU)(fA)(mC)(fA)(mC)(mU)(mU)(fA)#(mC)#(mA)-TegChol | 130 |
| INHBA_XM_017012174_4833 | (mC)#(mA)#(mA)(fU)(mC)(fU)(mU)(fA)(mG)(fA)(mA)(mG)(mC)(fU)#(mC)#(mA)-TegChol | 131 |
| INHBA_XM_017012174_5784 | (mA)#(mC)#(mU)(fA)(mU)(fG)(mU)(fC)(mA)(fA)(mU)(mG)(mG)(fU)#(mU)#(mA)-TegChol | 132 |
| INHBA_XM_017012174_5797 | (mU)#(mU)#(mU)(fC)(mU)(fU)(mA)(fA)(mU)(fA)(mU)(mU)(mU)(fA)#(mU)#(mA)-TegChol | 133 |
| INHBA_XM_017012174_4988 | (mC)#(mC)#(mA)(fA)(mU)(fU)(mU)(fC)(mA)(fU)(mA)(mU)(mU)(fU)#(mA)#(mA)-TegChol | 134 |
| INHBA_XM_017012174_5728 | (mC)#(mC)#(mA)(fA)(mU)(fG)(mU)(fC)(mU)(fG)(mU)(mU)(mU)(fG)#(mG)#(mA)-TegChol | 135 |
| INHBA_XM_017012174_3297 | (mU)#(mU)#(mA)(fA)(mA)(fU)(mC)(fA)(mG)(fA)(mA)(mG)(mC)(fU)#(mU)#(mA)-TegChol | 136 |
| INHBA_XM_017012174_5341 | (mU)#(mC)#(mA)(fC)(mU)(fG)(mA)(fG)(mA)(fA)(mG)(mC)(mU)(fG)#(mU)#(mA)-TegChol | 137 |
| INHBA_XM_017012174_3048 | (mG)#(mA)#(mA)(fG)(mG)(fA)(mU)(fA)(mG)(fG)(mC)(mU)(mA)(fU)#(mA)#(mA)-TegChol | 138 |
| INHBA_XM_017012174_2219 | (mC)#(mC)#(mA)(fA)(mG)(fU)(mU)(fA)(mC)(fA)(mU)(mU)(mU)(fC)#(mG)#(mA)-TegChol | 139 |
| INHBA_XM_017012174_5891 | (mG)#(mC)#(mA)(fG)(mA)(fA)(mG)(fU)(mU)(fA)(mU)(mU)(mU)(fA)#(mU)#(mA)-TegChol | 140 |
| Inhba_NM_008380_6118 | (mC)#(mU)#(mA)(fU)(mG)(fU)(mC)(fA)(mA)(fU)(mG)(mG)(mU)(fU)#(mU)#(mA)-TegChol | 141 |
| Inhba_NM_008380_5037 | (mA)#(mC)#(mA)(fA)(mU)(fA)(mU)(fU)(mA)(fG)(mA)(mU)(mA)(fU)#(mU)#(mA)-TegChol | 142 |
| Inhba_NM_008380_2941 | (mG)#(mA)#(mU)(fU)(mC)(fA)(mA)(fA)(mC)(fA)(mU)(mU)(mU)(fC)#(mU)#(mA)-TegChol | 143 |
| Inhba_NM_008380_2940 | (mG)#(mG)#(mA)(fU)(mU)(fC)(mA)(fA)(mA)(fC)(mA)(mU)(mU)(fU)#(mC)#(mA)-TegChol | 144 |
| Inhba_NM_008380_4144 | (mU)#(mC)#(mA)(fG)(mU)(fU)(mA)(fU)(mU)(fC)(mA)(mU)(mU)(fU)#(mA)#(mA)-TegChol | 145 |
| Inhba_NM_008380_5011 | (mA)#(mU)#(mA)(fG)(mA)(fA)(mU)(fU)(mU)(fA)(mA)(mA)(mU)(fU)#(mG)#(mA)-TegChol | 146 |
| Inhba_NM_008380_3138 | (mA)#(mU)#(mA)(fA)(mU)(fU)(mA)(fU)(mA)(fA)(mU)(mU)(mA)(fU)#(mU)#(mA)-TegChol | 147 |
| Inhba_NM_008380_2807 | (mC)#(mA)#(mA)(fC)(mC)(fU)(mA)(fA)(mU)(fA)(mG)(mG)(mU)(fU)#(mU)#(mA)-TegChol | 148 |
| Inhba_NM_008380_3507 | (mG)#(mC)#(mA)(fC)(mU)(fU)(mC)(fA)(mG)(fG)(mC)(mU)(mG)(fU)#(mC)#(mA)-TegChol | 149 |
| Inhba_NM_008380_4669 | (mC)#(mC)#(mC)(fU)(mC)(fU)(mG)(fA)(mA)(fC)(mU)(mG)(mU)(fU)#(mU)#(mA)-TegChol | 150 |
| Inhba_NM_008380_5038 | (mC)#(mA)#(mA)(fU)(mA)(fU)(mU)(fA)(mG)(fA)(mU)(mA)(mU)(fU)#(mG)#(mA)-TegChol | 151 |
| Inhba_NM_008380_6187 | (mA)#(mG)#(mA)(fG)(mA)(fU)(mA)(fA)(mC)(fG)(mG)(mU)(mU)(fG)#(mG)#(mA)-TegChol | 152 |
| Inhba_NM_008380_4287 | (mG)#(mC)#(mA)(fU)(mU)(fA)(mA)(fG)(mU)(fC)(mA)(mG)(mU)(fC)#(mC)#(mA)-TegChol | 153 |
| Inhba_NM_008380_4148 | (mU)#(mU)#(mA)(fU)(mU)(fC)(mA)(fU)(mU)(fU)(mA)(mC)(mA)(fU)#(mA)#(mA)-TegChol | 154 |
| Inhba_NM_008380_4302 | (mA)#(mU)#(mA)(fG)(mA)(fU)(mU)(fA)(mA)(fA)(mG)(mU)(mC)(fA)#(mU)#(mA)-TegChol | 155 |
| Inhba_NM_008380_5824 | (mA)#(mA)#(mG)(fU)(mG)(fA)(mA)(fU)(mC)(fA)(mU)(mU)(mU)(fC)#(mU)#(mA)-TegChol | 156 |
| Inhba_NM_008380_5006 | (mU)#(mU)#(mG)(fC)(mA)(fA)(mU)(fA)(mG)(fA)(mA)(mU)(mU)(fU)#(mA)#(mA)-TegChol | 157 |
| Inhba_NM_008380_3981 | (mG)#(mU)#(mU)(fG)(mU)(fA)(mC)(fA)(mA)(fA)(mU)(mC)(mU)(fC)#(mU)#(mA)-TegChol | 158 |
| Inhba_NM_008380_5900 | (mA)#(mC)#(mA)(fA)(mU)(fG)(mG)(fU)(mU)(fG)(mA)(mU)(mA)(fU)#(mU)#(mA)-TegChol | 159 |
| Inhba_NM_008380_6223 | (mG)#(mC)#(mA)(fG)(mA)(fA)(mG)(fU)(mU)(fA)(mU)(mU)(mU)(fA)#(mU)#(mA)-TegChol | 160 |
| Inhba_NM_008380_4413 | (mU)#(mC)#(mA)(fA)(mU)(fA)(mU)(fA)(mC)(fC)(mU)(mU)(mA)(fU)#(mC)#(mA)-TegChol | 161 |
| Inhba_NM_008380_4191 | (mA)#(mC)#(mU)(fU)(mG)(fU)(mU)(fU)(mG)(fA)(mA)(mG)(mU)(fU)#(mU)#(mA)-TegChol | 162 |
| Inhba_NM_008380_6078 | (mA)#(mA)#(mU)(fG)(mU)(fA)(mA)(fU)(mU)(fA)(mA)(mA)(mG)(fU)#(mU)#(mA)-TegChol | 163 |
| Inhba_NM_008380_5797 | (mC)#(mU)#(mA)(fU)(mA)(fU)(mU)(fA)(mU)(fU)(mA)(mU)(mU)(fU)#(mA)#(mA)-TegChol | 164 |
| Inhba_NM_008380_4535 | (mU)#(mA)#(mA)(fG)(mA)(fA)(mA)(fU)(mG)(fA)(mA)(mU)(mU)(fA)#(mU)#(mA)-TegChol | 165 |
| Inhba_NM_008380_3372 | (mG)#(mU)#(mA)(fG)(mU)(fG)(mU)(fA)(mC)(fA)(mC)(mU)(mG)(fU)#(mU)#(mA)-TegChol | 166 |
| Inhba_NM_008380_3141 | (mA)#(mU)#(mU)(fA)(mU)(fA)(mA)(fU)(mU)(fA)(mU)(mU)(mA)(fU)#(mU)#(mA)-TegChol | 167 |
| Inhba_NM_008380_3986 | (mA)#(mC)#(mA)(fA)(mA)(fU)(mC)(fU)(mC)(fU)(mA)(mA)(mU)(fA)#(mU)#(mA)-TegChol | 168 |
| Inhba_NM_008380_3450 | (mA)#(mU)#(mA)(fU)(mG)(fC)(mA)(fU)(mA)(fU)(mC)(mU)(mG)(fU)#(mA)#(mA)-TegChol | 169 |
| Inhba_NM_008380_3810 | (mA)#(mC)#(mU)(fU)(mA)(fU)(mA)(fU)(mU)(fU)(mA)(mU)(mU)(fA)#(mU)#(mA)-TegChol | 170 |
| Inhba_NM_008380_6318 | (mC)#(mA)#(mA)(fU)(mG)(fC)(mC)(fC)(mG)(fA)(mU)(mA)(mG)(fC)#(mU)#(mA)-TegChol | 171 |
| Inhba_NM_008380_6117 | (mA)#(mC)#(mU)(fA)(mU)(fG)(mU)(fC)(mA)(fA)(mU)(mG)(mG)(fU)#(mU)#(mA)-TegChol | 172 |
| Inhba_NM_008380_6130 | (mU)#(mU)#(mU)(fC)(mU)(fU)(mA)(fA)(mU)(fA)(mU)(mU)(mU)(fA)#(mU)#(mA)-TegChol | 173 |
| Inhba_NM_008380_4771 | (mG)#(mA)#(mA)(fG)(mA)(fG)(mC)(fA)(mG)(fG)(mA)(mU)(mU)(fU)#(mA)#(mA)-TegChol | 174 |
| Inhba_NM_008380_1767 | (mC)#(mC)#(mA)(fA)(mC)(fC)(mU)(fU)(mA)(fA)(mG)(mU)(mC)(fA)#(mU)#(mA)-TegChol | 175 |
| Inhba_NM_008380_5895 | (mC)#(mA)#(mU)(fU)(mG)(fA)(mC)(fA)(mA)(fU)(mG)(mG)(mU)(fU)#(mG)#(mA)-TegChol | 176 |
| Inhba_NM_008380_6079 | (mA)#(mU)#(mG)(fU)(mA)(fA)(mU)(fU)(mA)(fA)(mA)(mG)(mU)(fU)#(mG)#(mA)-TegChol | 177 |
| Inhba_NM_008380_3578 | (mA)#(mG)#(mA)(fA)(mG)(fG)(mA)(fU)(mA)(fA)(mG)(mU)(mG)(fA)#(mU)#(mA)-TegChol | 178 |
| Inhba_NM_008380_2405 | (mA)#(mU)#(mG)(fU)(mG)(fA)(mC)(fA)(mC)(fU)(mA)(mU)(mC)(fU)#(mC)#(mA)-TegChol | 179 |
| Inhba_NM_008380_2764 | (mC)#(mC)#(mA)(fA)(mG)(fU)(mU)(fA)(mC)(fA)(mU)(mU)(mU)(fC)#(mG)#(mA)-TegChol | 180 |
| Inhba_NM_008380_2915 | (mA)#(mA)#(mA)(fG)(mG)(fA)(mA)(fU)(mA)(fU)(mG)(mU)(mU)(fU)#(mG)#(mA)-TegChol | 181 |
| Inhba_NM_008380_6218 | (mU)#(mU)#(mA)(fU)(mA)(fG)(mC)(fA)(mG)(fA)(mA)(mG)(mU)(fU)#(mA)#(mA)-TegChol | 182 |
| Inhba_NM_008380_5004 | (mC)#(mC)#(mU)(fU)(mG)(fC)(mA)(fA)(mU)(fA)(mG)(mA)(mA)(fU)#(mU)#(mA)-TegChol | 183 |
| Inhba_NM_008380_3013 | (mA)#(mC)#(mC)(fU)(mG)(fU)(mC)(fU)(mA)(fA)(mU)(mC)(mC)(fU)#(mU)#(mA)-TegChol | 184 |
| Inhba_NM_008380_6066 | (mC)#(mA)#(mA)(fU)(mG)(fU)(mC)(fU)(mG)(fG)(mC)(mU)(mA)(fA)#(mU)#(mA)-TegChol | 185 |
| Inhba_NM_008380_5511 | (mA)#(mC)#(mA)(fU)(mG)(fC)(mC)(fU)(mC)(fA)(mG)(mU)(mU)(fA)#(mU)#(mA)-TegChol | 186 |
| Inhba_NM_008380_5049 | (mA)#(mU)#(mU)(fG)(mU)(fU)(mA)(fA)(mC)(fA)(mA)(mU)(mU)(fU)#(mA)#(mA)-TegChol | 187 |
| Inhba_NM_008380_6149 | (mU)#(mA)#(mG)(fA)(mA)(fG)(mU)(fG)(mC)(fU)(mG)(mG)(mU)(fA)#(mA)#(mA)-TegChol | 188 |
| | AntisenseStrand | |
| INHBA_XM_017012174_5785 | P(mU)#(fA)#(mA)(fA)(fC)(fC)(mA)(fU)(mU)(fG)(mA)(fC)(mA)(fU)#(mA)#(fG)#(mU)#(mU)#(mG)#(mU)#(fU) | 189 |
| INHBA_XM_017012174_4169 | P(mU)#(fU)#(mA)(fA)(fA)(fG)(mU)(fU)(mG)(fA)(mA)(fC)(mA)(fU)#(mG)#(fG)#(mC)#(mC)#(mA)#(mU)#(fU) | 190 |
| INHBA_XM_017012174_5169 | P(mU)#(fU)#(mG)(fA)(fA)(fU)(mA)(fU)(mG)(fA)(mU)(fA)(mA)(fU)#(mA)#(fU)#(mG)#(mG)#(mG)#(mU)#(fU) | 191 |
| INHBA_XM_017012174_3792 | P(mU)#(fG)#(mU)(fA)(fA)(fU)(mU)(fG)(mU)(fU)(mC)(fC)(mC)(fA)#(mA)#(fU)#(mU)#(mA)#(mU)#(mU)#(fU) | 192 |
| INHBA_XM_017012174_3843 | P(mU)#(fC)#(mA)(fA)(fA)(fU)(mU)(fG)(mA)(fC)(mA)(fU)(mA)(fG)#(mG)#(fG)#(mC)#(mU)#(mA)#(mU)#(fU) | 193 |
| INHBA_XM_017012174_2395 | P(mU)#(fG)#(mA)(fA)(fA)(fU)(mG)(fU)(mU)(fU)(mG)(fG)(mA)(fU)#(mC)#(fC)#(mA)#(mA)#(mC)#(mU)#(fU) | 194 |
| INHBA_XM_017012174_5857 | P(mU)#(fA)#(mU)(fC)(fA)(fA)(mA)(fC)(mU)(fG)(mU)(fU)(mA)(fU)#(mC)#(fU)#(mC)#(mU)#(mU)#(mU)#(fU) | 195 |
| INHBA_XM_017012174_5167 | P(mU)#(fA)#(mA)(fU)(fA)(fU)(mG)(fA)(mU)(fA)(mA)(fU)(mA)(fU)#(mG)#(fG)#(mG)#(mU)#(mC)#(mU)#(fU) | 196 |
| INHBA_XM_017012174_4722 | P(mU)#(fA)#(mU)(fA)(fC)(fU)(mA)(fC)(mU)(fA)(mA)(fG)(mA)(fA)#(mU)#(fC)#(mC)#(mC)#(mA)#(mU)#(fU) | 197 |
| INHBA_XM_017012174_3844 | P(mU)#(fA)#(mC)(fA)(fA)(fA)(mU)(fU)(mG)(fA)(mC)(fA)(mU)(fA)#(mG)#(fG)#(mG)#(mC)#(mU)#(mU)#(fU) | 198 |
| INHBA_XM_017012174_4519 | P(mU)#(fG)#(mU)(fG)(fG)(fG)(mU)(fG)(mU)(fU)(mU)(fG)(mA)(fU)#(mA)#(fA)#(mC)#(mC)#(mC)#(mU)#(fU) | 199 |
| INHBA_XM_017012174_3799 | P(mU)#(fA)#(mA)(fU)(fU)(fA)(mA)(fU)(mG)(fU)(mA)(fA)(mU)(fU)#(mG)#(fU)#(mU)#(mC)#(mC)#(mU)#(fU) | 200 |
| INHBA_XM_017012174_3789 | P(mU)#(fA)#(mU)(fU)(fG)(fU)(mU)(fC)(mC)(fC)(mA)(fA)(mU)(fU)#(mA)#(fU)#(mU)#(mA)#(mG)#(mU)#(fU) | 201 |
| INHBA_XM_017012174_5059 | P(mU)#(fA)#(mA)(fG)(fU)(fA)(mU)(fU)(mU)(fG)(mU)(fU)(mC)(fU)#(mA)#(fC)#(mC)#(mU)#(mU)#(mU)#(fU) | 202 |
| INHBA_XM_017012174_5892 | P(mU)#(fA)#(mA)(fU)(fA)(fA)(mA)(fU)(mA)(fA)(mC)(fU)(mU)(fC)#(mU)#(fG)#(mC)#(mU)#(mA)#(mU)#(fU) | 203 |
| INHBA_XM_017012174_2977 | P(mU)#(fG)#(mA)(fC)(fA)(fG)(mC)(fC)(mU)(fG)(mA)(fA)(mG)(fU)#(mG)#(fC)#(mU)#(mU)#(mA)#(mU)#(fU) | 204 |
| INHBA_XM_017012174_4714 | P(mU)#(fA)#(mA)(fG)(fA)(fA)(mU)(fC)(mC)(fC)(mA)(fU)(mA)(fU)#(mG)#(fU)#(mU)#(mA)#(mU)#(mU)#(fU) | 205 |
| INHBA_XM_017012174_2262 | P(mU)#(fA)#(mA)(fA)(fC)(fC)(mU)(fA)(mU)(fU)(mA)(fG)(mG)(fU)#(mU)#(fG)#(mC)#(mA)#(mU)#(mU)#(fU) | 206 |
| INHBA_XM_017012174_5419 | P(mU)#(fA)#(mU)(fA)(fG)(fC)(mU)(fG)(mA)(fG)(mA)(fC)(mA)(fU)#(mG)#(fU)#(mG)#(mG)#(mU)#(mU)#(fU) | 207 |
| INHBA_XM_017012174_2188 | P(mU)#(fA)#(mU)(fC)(fU)(fU)(mU)(fC)(mA)(fU)(mU)(fC)(mU)(fU)#(mU)#(fC)#(mC)#(mA)#(mU)#(mU)#(fU) | 208 |
| INHBA_XM_017012174_556 | P(mU)#(fU)#(mU)(fC)(fA)(fU)(mU)(fC)(mA)(fU)(mU)(fU)(mC)(fU)#(mG)#(fC)#(mC)#(mC)#(mU)#(mU)#(fU) | 209 |
| INHBA_XM_017012174_1418 | P(mU)#(fA)#(mC)(fA)(fA)(fC)(mA)(fU)(mG)(fG)(mA)(fC)(mA)(fU)#(mG)#(fG)#(mG)#(mU)#(mC)#(mU)#(fU) | 210 |
| INHBA_XM_017012174_4878 | P(mU)#(fA)#(mA)(fU)(fA)(fC)(mA)(fU)(mG)(fU)(mU)(fA)(mC)(fU)#(mA)#(fU)#(mU)#(mA)#(mA)#(mU)#(fU) | 211 |
| INHBA_XM_017012174_4562 | P(mU)#(fA)#(mC)(fU)(fU)(fC)(mU)(fA)(mU)(fU)(mU)(fC)(mA)(fA)#(mA)#(fG)#(mC)#(mC)#(mA)#(mU)#(fU) | 212 |
| INHBA_XM_017012174_5854 | P(mU)#(fA)#(mA)(fA)(fC)(fU)(mG)(fU)(mU)(fA)(mU)(fC)(mU)(fC)#(mU)#(fU)#(mU)#(mA)#(mA)#(mU)#(fU) | 213 |
| INHBA_XM_017012174_5535 | P(mU)#(fA)#(mA)(fU)(fA)(fU)(mC)(fA)(mA)(fC)(mC)(fC)(mA)(fU)#(mU)#(fG)#(mU)#(mC)#(mA)#(mU)#(fU) | 214 |
| INHBA_XM_017012174_2839 | P(mU)#(fA)#(mA)(fC)(fA)(fG)(mU)(fG)(mU)(fA)(mC)(fA)(mA)(fC)#(mU)#(fA)#(mC)#(mU)#(mG)#(mU)#(fU) | 215 |
| INHBA_XM_017012174_5886 | P(mU)#(fU)#(mA)(fA)(fC)(fU)(mU)(fC)(mU)(fG)(mC)(fU)(mA)(fU)#(mA)#(fA)#(mA)#(mC)#(mA)#(mU)#(fU) | 216 |
| INHBA_XM_017012174_4155 | P(mU)#(fG)#(mC)(fC)(fA)(fU)(mC)(fU)(mA)(fC)(mU)(fC)(mU)(fU)#(mG)#(fC)#(mC)#(mU)#(mU)#(mU)#(fU) | 217 |
| INHBA_XM_017012174_2918 | P(mU)#(fC)#(mA)(fG)(fA)(fU)(mA)(fU)(mG)(fC)(mA)(fU)(mA)(fU)#(mG)#(fC)#(mA)#(mC)#(mA)#(mU)#(fU) | 218 |
| INHBA_XM_017012174_5587 | P(mU)#(fA)#(mC)(fA)(fG)(fG)(mC)(fU)(mU)(fC)(mA)(fG)(mA)(fU)#(mU)#(fA)#(mU)#(mC)#(mU)#(mU)#(fU) | 219 |
| INHBA_XM_017012174_1579 | P(mU)#(fU)#(mU)(fA)(fA)(fC)(mU)(fC)(mA)(fG)(mA)(fA)(mA)(fC)#(mC)#(fU)#(mU)#(mA)#(mA)#(mU)#(fU) | 220 |
| INHBA_XM_017012174_2396 | P(mU)#(fA)#(mG)(fA)(fA)(fA)(mU)(fG)(mU)(fU)(mU)(fG)(mG)(fA)#(mU)#(fC)#(mC)#(mA)#(mA)#(mU)#(fU) | 221 |
| INHBA_XM_017012174_4449 | P(mU)#(fA)#(mU)(fU)(fA)(fC)(mA)(fU)(mU)(fU)(mC)(fU)(mC)(fU)#(mG)#(fC)#(mU)#(mU)#(mC)#(mU)#(fU) | 222 |
| INHBA_XM_017012174_2320 | P(mU)#(fA)#(mG)(fA)(fU)(fU)(mA)(fC)(mU)(fU)(mC)(fC)(mA)(fA)#(mU)#(fA)#(mC)#(mA)#(mA)#(mU)#(fU) | 223 |
| INHBA_XM_017012174_2920 | P(mU)#(fU)#(mA)(fC)(fA)(fG)(mA)(fU)(mA)(fU)(mG)(fC)(mA)(fU)#(mA)#(fU)#(mG)#(mC)#(mA)#(mU)#(fU) | 224 |
| INHBA_XM_017012174_2321 | P(mU)#(fC)#(mA)(fG)(fA)(fU)(mU)(fA)(mC)(fU)(mU)(fC)(mC)(fA)#(mA)#(fU)#(mA)#(mC)#(mA)#(mU)#(fU) | 225 |
| INHBA_XM_017012174_3999 | P(mU)#(fG)#(mU)(fA)(fA)(fG)(mU)(fG)(mU)(fA)(mU)(fU)(mU)(fG)#(mC)#(fC)#(mC)#(mA)#(mG)#(mU)#(fU) | 226 |
| INHBA_XM_017012174_4833 | P(mU)#(fG)#(mA)(fG)(fC)(fU)(mU)(fC)(mU)(fA)(mA)(fG)(mA)(fU)#(mU)#(fG)#(mG)#(mU)#(mG)#(mU)#(fU) | 227 |
| INHBA_XM_017012174_5784 | P(mU)#(fA)#(mA)(fC)(fC)(fA)(mU)(fU)(mG)(fA)(mC)(fA)(mU)(fA)#(mG)#(fU)#(mU)#(mG)#(mA)#(mU)#(fU) | 228 |
| INHBA_XM_017012174_5797 | P(mU)#(fA)#(mU)(fA)(fA)(fA)(mU)(fA)(mU)(fU)(mA)(fA)(mG)(fA)#(mA)#(fA)#(mC)#(mC)#(mA)#(mU)#(fU) | 229 |
| INHBA_XM_017012174_4988 | P(mU)#(fU)#(mA)(fA)(fA)(fU)(mA)(fU)(mG)(fA)(mA)(fA)(mU)(fU)#(mG)#(fG)#(mU)#(mU)#(mG)#(mU)#(fU) | 230 |
| INHBA_XM_017012174_5728 | P(mU)#(fC)#(mC)(fA)(fA)(fA)(mC)(fA)(mG)(fA)(mC)(fA)(mU)(fU)#(mG)#(fG)#(mU)#(mU)#(mA)#(mU)#(fU) | 231 |
| INHBA_XM_017012174_3297 | P(mU)#(fA)#(mA)(fG)(fC)(fU)(mU)(fC)(mU)(fG)(mA)(fU)(mU)(fU)#(mA)#(fA)#(mG)#(mA)#(mU)#(mU)#(fU) | 232 |
| INHBA_XM_017012174_5341 | P(mU)#(fA)#(mC)(fA)(fG)(fC)(mU)(fU)(mC)(fU)(mC)(fA)(mG)(fU)#(mG)#(fA)#(mU)#(mA)#(mC)#(mU)#(fU) | 233 |
| INHBA_XM_017012174_3048 | P(mU)#(fU)#(mA)(fU)(fA)(fG)(mC)(fC)(mU)(fA)(mU)(fC)(mC)(fU)#(mU)#(fC)#(mU)#(mU)#(mA)#(mU)#(fU) | 234 |
| INHBA_XM_017012174_2219 | P(mU)#(fC)#(mG)(fA)(fA)(fA)(mU)(fG)(mU)(fA)(mA)(fC)(mU)(fU)#(mG)#(fG)#(mU)#(mA)#(mU)#(mU)#(fU) | 235 |
| INHBA_XM_017012174_5891 | P(mU)#(fA)#(mU)(fA)(fA)(fA)(mU)(fA)(mA)(fC)(mU)(fU)(mC)(fU)#(mG)#(fC)#(mU)#(mA)#(mU)#(mU)#(fU) | 236 |
| Inhba_NM_008380_6118 | P(mU)#(fA)#(mA)(fA)(fC)(fC)(mA)(fU)(mU)(fG)(mA)(fC)(mA)(fU)#(mA)#(fG)#(mU)#(mU)#(mG)#(mU)#(fU) | 237 |
| Inhba_NM_008380_5037 | P(mU)#(fA)#(mA)(fU)(fA)(fU)(mC)(fU)(mA)(fA)(mU)(fA)(mU)(fU)#(mG)#(fU)#(mU)#(mA)#(mA)#(mU)#(fU) | 238 |
| Inhba_NM_008380_2941 | P(mU)#(fA)#(mG)(fA)(fA)(fA)(mU)(fG)(mU)(fU)(mU)(fG)(mA)(fA)#(mU)#(fC)#(mC)#(mA)#(mA)#(mU)#(fU) | 239 |
| Inhba_NM_008380_2940 | P(mU)#(fG)#(mA)(fA)(fA)(fU)(mG)(fU)(mU)(fU)(mG)(fA)(mA)(fU)#(mC)#(fC)#(mA)#(mA)#(mC)#(mU)#(fU) | 240 |
| Inhba_NM_008380_4144 | P(mU)#(fU)#(mA)(fA)(fA)(fU)(mG)(fA)(mA)(fU)(mA)(fA)(mC)(fU)#(mG)#(fA)#(mU)#(mU)#(mA)#(mU)#(fU) | 241 |
| Inhba_NM_008380_5011 | P(mU)#(fC)#(mA)(fA)(fU)(fU)(mU)(fA)(mA)(fA)(mU)(fU)(mC)(fU)#(mA)#(fU)#(mU)#(mG)#(mC)#(mU)#(fU) | 242 |
| Inhba_NM_008380_3138 | P(mU)#(fA)#(mA)(fU)(fA)(fA)(mU)(fU)(mA)(fU)(mA)(fA)(mU)(fU)#(mA)#(fU)#(mA)#(mA)#(mU)#(mU)#(fU) | 243 |
| Inhba_NM_008380_2807 | P(mU)#(fA)#(mA)(fA)(fC)(fC)(mU)(fA)(mU)(fU)(mA)(fG)(mG)(fU)#(mU)#(fG)#(mC)#(mA)#(mU)#(mU)#(fU) | 244 |
| Inhba_NM_008380_3507 | P(mU)#(fG)#(mA)(fC)(fA)(fG)(mC)(fC)(mU)(fG)(mA)(fA)(mG)(fU)#(mG)#(fC)#(mU)#(mU)#(mA)#(mU)#(fU) | 245 |
| Inhba_NM_008380_4669 | P(mU)#(fA)#(mA)(fA)(fC)(fA)(mG)(fU)(mU)(fC)(mA)(fG)(mA)(fG)#(mG)#(fG)#(mC)#(mG)#(mC)#(mU)#(fU) | 246 |
| Inhba_NM_008380_5038 | P(mU)#(fC)#(mA)(fA)(fU)(fA)(mU)(fC)(mU)(fA)(mA)(fU)(mA)(fU)#(mU)#(fG)#(mU)#(mU)#(mA)#(mU)#(fU) | 247 |
| Inhba_NM_008380_6187 | P(mU)#(fC)#(mC)(fA)(fA)(fC)(mC)(fG)(mU)(fU)(mA)(fU)(mC)(fU)#(mC)#(fU)#(mU)#(mU)#(mA)#(mU)#(fU) | 248 |
| Inhba_NM_008380_4287 | P(mU)#(fG)#(mG)(fA)(fC)(fU)(mG)(fA)(mC)(fU)(mU)(fA)(mA)(fU)#(mG)#(fC)#(mU)#(mA)#(mA)#(mU)#(fU) | 249 |
| Inhba_NM_008380_4148 | P(mU)#(fU)#(mA)(fU)(fG)(fU)(mA)(fA)(mA)(fU)(mG)(fA)(mA)(fU)#(mA)#(fA)#(mC)#(mU)#(mG)#(mU)#(fU) | 250 |
| Inhba_NM_008380_4302 | P(mU)#(fA)#(mU)(fG)(fA)(fC)(mU)(fU)(mU)(fA)(mA)(fU)(mC)(fU)#(mA)#(fU)#(mG)#(mG)#(mA)#(mU)#(fU) | 251 |
| Inhba_NM_008380_5824 | P(mU)#(fA)#(mG)(fA)(fA)(fA)(mU)(fG)(mA)(fU)(mU)(fC)(mA)(fC)#(mU)#(fU)#(mU)#(mA)#(mU)#(mU)#(fU) | 252 |
| Inhba_NM_008380_5006 | P(mU)#(fU)#(mA)(fA)(fA)(fU)(mU)(fC)(mU)(fA)(mU)(fU)(mG)(fC)#(mA)#(fA)#(mG)#(mG)#(mC)#(mU)#(fU) | 253 |
| Inhba_NM_008380_3981 | P(mU)#(fA)#(mG)(fA)(fG)(fA)(mU)(fU)(mU)(fG)(mU)(fA)(mC)(fA)#(mA)#(fC)#(mU)#(mC)#(mC)#(mU)#(fU) | 254 |
| Inhba_NM_008380_5900 | P(mU)#(fA)#(mA)(fU)(fA)(fU)(mC)(fA)(mA)(fC)(mC)(fA)(mU)(fU)#(mG)#(fU)#(mC)#(mA)#(mA)#(mU)#(fU) | 255 |
| Inhba_NM_008380_6223 | P(mU)#(fA)#(mU)(fA)(fA)(fA)(mU)(fA)(mA)(fC)(mU)(fU)(mC)(fU)#(mG)#(fC)#(mU)#(mA)#(mU)#(mU)#(fU) | 256 |
| Inhba_NM_008380_4413 | P(mU)#(fG)#(mA)(fU)(fA)(fA)(mG)(fG)(mU)(fA)(mU)(fA)(mU)(fU)#(mG)#(fA)#(mC)#(mG)#(mG)#(mU)#(fU) | 257 |
| Inhba_NM_008380_4191 | P(mU)#(fA)#(mA)(fA)(fC)(fU)(mU)(fC)(mA)(fA)(mA)(fC)(mA)(fA)#(mG)#(fU)#(mC)#(mC)#(mG)#(mU)#(fU) | 258 |
| Inhba_NM_008380_6078 | P(mU)#(fA)#(mA)(fC)(fU)(fU)(mU)(fA)(mA)(fU)(mU)(fA)(mC)(fA)#(mU)#(fU)#(mA)#(mG)#(mC)#(mU)#(fU) | 259 |
| Inhba_NM_008380_5797 | P(mU)#(fU)#(mA)(fA)(fA)(fU)(mA)(fA)(mU)(fA)(mA)(fU)(mA)(fU)#(mA)#(fG)#(mC)#(mU)#(mG)#(mU)#(fU) | 260 |
| Inhba_NM_008380_4535 | P(mU)#(fA)#(mU)(fA)(fA)(fU)(mU)(fC)(mA)(fU)(mU)(fU)(mC)(fU)#(mU)#(fA)#(mU)#(mG)#(mC)#(mU)#(fU) | 261 |
| Inhba_NM_008380_3372 | P(mU)#(fA)#(mA)(fC)(fA)(fG)(mU)(fG)(mU)(fA)(mC)(fA)(mC)(fU)#(mA)#(fC)#(mU)#(mG)#(mU)#(mU)#(fU) | 262 |
| Inhba_NM_008380_3141 | P(mU)#(fA)#(mA)(fU)(fA)(fA)(mU)(fA)(mA)(fU)(mU)(fA)(mU)(fA)#(mA)#(fU)#(mU)#(mA)#(mU)#(mU)#(fU) | 263 |
| Inhba_NM_008380_3986 | P(mU)#(fA)#(mU)(fA)(fU)(fU)(mA)(fG)(mA)(fG)(mA)(fU)(mU)(fU)#(mG)#(fU)#(mA)#(mC)#(mA)#(mU)#(fU) | 264 |
| Inhba_NM_008380_3450 | P(mU)#(fU)#(mA)(fC)(fA)(fG)(mA)(fU)(mA)(fU)(mG)(fC)(mA)(fU)#(mA)#(fU)#(mG)#(mC)#(mA)#(mU)#(fU) | 265 |
| Inhba_NM_008380_3810 | P(mU)#(fA)#(mU)(fA)(fA)(fU)(mA)(fA)(mA)(fU)(mA)(fU)(mA)(fA)#(mG)#(fU)#(mU)#(mG)#(mA)#(mU)#(fU) | 266 |
| Inhba_NM_008380_6318 | P(mU)#(fA)#(mG)(fC)(fU)(fA)(mU)(fC)(mG)(fG)(mG)(fC)(mA)(fU)#(mU)#(fG)#(mC)#(mU)#(mG)#(mU)#(fU) | 267 |
| Inhba_NM_008380_6117 | P(mU)#(fA)#(mA)(fC)(fC)(fA)(mU)(fU)(mG)(fA)(mC)(fA)(mU)(fA)#(mG)#(fU)#(mU)#(mG)#(mA)#(mU)#(fU) | 268 |
| Inhba_NM_008380_6130 | P(mU)#(fA)#(mU)(fA)(fA)(fA)(mU)(fA)(mU)(fU)(mA)(fA)(mG)(fA)#(mA)#(fA)#(mC)#(mC)#(mA)#(mU)#(fU) | 269 |
| Inhba_NM_008380_4771 | P(mU)#(fU)#(mA)(fA)(fA)(fU)(mC)(fC)(mU)(fG)(mC)(fU)(mC)(fU)#(mU)#(fC)#(mC)#(mC)#(mA)#(mU)#(fU) | 270 |
| Inhba_NM_008380_1767 | P(mU)#(fA)#(mU)(fG)(fA)(fC)(mU)(fU)(mA)(fA)(mG)(fG)(mU)(fU)#(mG)#(fG)#(mC)#(mA)#(mA)#(mU)#(fU) | 271 |
| Inhba_NM_008380_5895 | P(mU)#(fC)#(mA)(fA)(fC)(fC)(mA)(fU)(mU)(fG)(mU)(fC)(mA)(fA)#(mU)#(fG)#(mC)#(mA)#(mC)#(mU)#(fU) | 272 |
| Inhba_NM_008380_6079 | P(mU)#(fC)#(mA)(fA)(fC)(fU)(mU)(fU)(mA)(fA)(mU)(fU)(mA)(fC)#(mA)#(fU)#(mU)#(mA)#(mG)#(mU)#(fU) | 273 |
| Inhba_NM_008380_3578 | P(mU)#(fA)#(mU)(fC)(fA)(fC)(mU)(fU)(mA)(fU)(mC)(fC)(mU)(fU)#(mC)#(fU)#(mU)#(mA)#(mA)#(mU)#(fU) | 274 |
| Inhba_NM_008380_2405 | P(mU)#(fG)#(mA)(fG)(fA)(fU)(mA)(fG)(mU)(fG)(mU)(fC)(mA)(fC)#(mA)#(fU)#(mA)#(mG)#(mA)#(mU)#(fU) | 275 |
| Inhba_NM_008380_2764 | P(mU)#(fC)#(mG)(fA)(fA)(fA)(mU)(fG)(mU)(fA)(mA)(fC)(mU)(fU)#(mG)#(fG)#(mU)#(mA)#(mU)#(mU)#(fU) | 276 |
| Inhba_NM_008380_2915 | P(mU)#(fC)#(mA)(fA)(fA)(fC)(mA)(fU)(mA)(fU)(mU)(fC)(mC)(fU)#(mU)#(fU)#(mC)#(mA)#(mA)#(mU)#(fU) | 277 |
| Inhba_NM_008380_6218 | P(mU)#(fU)#(mA)(fA)(fC)(fU)(mU)(fC)(mU)(fG)(mC)(fU)(mA)(fU)#(mA)#(fA)#(mA)#(mC)#(mA)#(mU)#(fU) | 278 |
| Inhba_NM_008380_5004 | P(mU)#(fA)#(mA)(fU)(fU)(fC)(mU)(fA)(mU)(fU)(mG)(fC)(mA)(fA)#(mG)#(fG)#(mC)#(mC)#(mA)#(mU)#(fU) | 279 |
| Inhba_NM_008380_3013 | P(mU)#(fA)#(mA)(fG)(fG)(fA)(mU)(fU)(mA)(fG)(mA)(fC)(mA)(fG)#(mG)#(fU)#(mC)#(mA)#(mA)#(mU)#(fU) | 280 |
| Inhba_NM_008380_6066 | P(mU)#(fA)#(mU)(fU)(fA)(fG)(mC)(fC)(mA)(fG)(mA)(fC)(mA)(fU)#(mU)#(fG)#(mG)#(mU)#(mU)#(mU)#(fU) | 281 |
| Inhba_NM_008380_5511 | P(mU)#(fA)#(mU)(fA)(fA)(fC)(mU)(fG)(mA)(fG)(mG)(fC)(mA)(fU)#(mG)#(fU)#(mU)#(mU)#(mG)#(mU)#(fU) | 282 |
| Inhba_NM_008380_5049 | P(mU)#(fU)#(mA)(fA)(fA)(fU)(mU)(fG)(mU)(fU)(mA)(fA)(mC)(fA)#(mA)#(fU)#(mA)#(mU)#(mC)#(mU)#(fU) | 283 |
| Inhba_NM_008380_6149 | P(mU)#(fU)#(mU)(fA)(fC)(fC)(mA)(fG)(mC)(fA)(mC)(fU)(mU)(fC)#(mU)#(fA)#(mC)#(mA)#(mA)#(mU)#(fU) | 284 |

m is2′-O-methyl nucleosides;
f is 2′-Fluoro nucleosides;
P is 5′-(E)-Vinylphosphonate;
# is phosphorothioate linkage;
TegChol is TEG linker + cholesterol

In some embodiments, INHBA specific siRNA comprises an anti-sense strand that comprises the nucleic acid sequence with chemical modification pattern set forth in Table 3. In some embodiments, INHBA specific siRNA comprises an anti-sense strand that comprises the nucleic acid sequence with chemical modification pattern as set forth in SEQ ID NOs: 189-284. In some embodiments, INHBA specific siRNA comprises a sense strand that comprises the nucleic acid sequence with chemical modification pattern set forth in Table3. In some embodiments, INHBA specific siRNA comprises a sense strand that comprises the nucleic acid sequence with chemical modification pattern as set forth in SEQ ID NOs: 93-188.

iii. Human and Mouse Myostatin (MSTN) Specific siRNAs

In some aspects, an siRNA described herein specifically targets MSTN nucleic acid sequence (e.g., MSTN gene and/or MSTN mRNA). In some embodiments, an siRNA described herein specifically targets mouse and/or MSTN nucleic acid sequence (referred to as MSTN specific siRNA). Exemplary MSTN target gene region sequences and corresponding target sequences are set forth in Tables 11 and 12.

TABLE 11
Human MSTN target gene regions sequences and target sequences

| Accession | | | SEQ | | SEQ |
|---|---|---|---|---|---|
| number | Position | Target sequence | ID NO | Gene region | ID NO |
| NM_005259 | 246 | UGUGUAAUGCAUG | 1473 | TGGAAAAAGAGGGGCTGTGTAAT | 1376 |
| | | UACUUGG | | GCATGTACTTGGAGACAAAACA | |
| NM_005259 | 284 | UCAAGAAUAGAAG | 1474 | CAAAACACTAAATCTTCAAGAAT | 1377 |
| | | CCAUUAA | | AGAAGCCATTAAGATACAAATC | |
| NM_005259 | 296 | GCCAUUAAGAUAC | 1475 | TCTTCAAGAATAGAAGCCATTAA | 1378 |
| | | AAAUCCU | | GATACAAATCCTCAGTAAACTT | |
| NM_005259 | 344 | AACAUCAGCAAAG | 1476 | CTGGAAACAGCTCCTAACATCAG | 1379 |
| | | AUGUUAU | | CAAAGATGTTATAAGACAACTT | |
| NM_005259 | 542 | UGUUGCUUCUUUA | 1477 | GATGGAAAACCCAAATGTTGCTT | 1380 |
| | | AAUUUAG | | CTTTAAATTTAGCTCTAAAATA | |
| NM_005259 | 585 | UAGUAAAGGCCCA | 1478 | TACAATACAATAAAGTAGTAAAG | 1381 |
| | | ACUAUGG | | GCCCAACTATGGATATATTTGA | |
| NM_005259 | 622 | CGAGACUCCUACA | 1479 | ATATTTGAGACCCGTCGAGACTC | 1382 |
| | | ACAGUGU | | CTACAACAGTGTTTGTGCAAAT | |
| NM_005259 | 626 | ACUCCUACAACAG | 1480 | TTGAGACCCGTCGAGACTCCTAC | 1383 |
| | | UGUUUGU | | AACAGTGTTTGTGCAAATCCTG | |
| NM_005259 | 752 | GAUGUGAAGACAG | 1481 | ATTTGGCAGAGCATTGATGTGAA | 1384 |
| | | UGUUGCA | | GACAGTGTTGCAAAATTGGCTC | |
| NM_005259 | 959 | UCAACAGAAUCAC | 1482 | GACTGTGATGAGCACTCAACAGA | 1385 |
| | | GAUGCUG | | ATCACGATGCTGTCGTTACCCT | |
| NM_005259 | 1011 | UUGGAUGGGAUUG | 1483 | TGGATTTTGAAGCTTTTGGATGG | 1386 |
| | | GAUUAUC | | GATTGGATTATCGCTCCTAAAA | |
| NM_005259 | 1015 | AUGGGAUUGGAUU | 1484 | TTTTGAAGCTTTTGGATGGGATT | 1387 |
| | | AUCGCUC | | GGATTATCGCTCCTAAAAGATA | |
| NM_005259 | 1055 | UACUGCUCUGGAG | 1485 | AGATATAAGGCCAATTACTGCTC | 1388 |
| | | AGUGUGA | | TGGAGAGTGTGAATTTGTATTT | |
| NM_005259 | 1061 | UCUGGAGAGUGUG | 1486 | AAGGCCAATTACTGCTCTGGAGA | 1389 |
| | | AAUUUGU | | GTGTGAATTTGTATTTTTACAA | |
| NM_005259 | 1062 | CUGGAGAGUGUGA | 1487 | AGGCCAATTACTGCTCTGGAGAG | 1390 |
| | | AUUUGUA | | TGTGAATTTGTATTTTTACAAA | |
| NM_005259 | 1168 | GUCUCCAAUUAAU | 1488 | TACTCCCACAAAGATGTCTCCAA | 1391 |
| | | AUGCUAU | | TTAATATGCTATATTTTAATGG | |
| NM_005259 | 1169 | UCUCCAAUUAAUA | 1489 | ACTCCCACAAAGATGTCTCCAAT | 1392 |
| | | UGCUAUA | | TAATATGCTATATTTTAATGGC | |
| NM_005259 | 1172 | CCAAUUAAUAUGC | 1490 | CCCACAAAGATGTCTCCAATTAA | 1393 |
| | | UAUAUUU | | TATGCTATATTTTAATGGCAAA | |
| NM_005259 | 1193 | AAUGGCAAAGAAC | 1491 | AATATGCTATATTTTAATGGCAA | 1394 |
| | | AAAUAAU | | AGAACAAATAATATATGGGAAA | |
| NM_005259 | 1194 | AUGGCAAAGAACA | 1492 | ATATGCTATATTTTAATGGCAAA | 1395 |
| | | AAUAAUA | | GAACAAATAATATATGGGAAAA | |
| NM_005259 | 1196 | GGCAAAGAACAAA | 1493 | ATGCTATATTTTAATGGCAAAGA | 1396 |
| | | UAAUAUA | | ACAAATAATATATGGGAAAATT | |
| NM_005259 | 1377 | UAAGCAUAAGCUA | 1494 | TATGCTACAGTCACTTAAGCATA | 1397 |
| | | CAGUAUG | | AGCTACAGTATGTAAACTAAAA | |
| NM_005259 | 1409 | GGGAAUAUAUGCA | 1495 | ATGTAAACTAAAAGGGGGAATAT | 1398 |
| | | AUGGUUG | | ATGCAATGGTTGGCATTTAACC | |
| NM_005259 | 1464 | UUUAUGAUUUCCA | 1496 | ATCATACAAGAAAGTTTTATGAT | 1399 |
| | | GAGUUUU | | TTCCAGAGTTTTTGAGCTAGAA | |
| NM_005259 | 1482 | UUUGAGCUAGAAG | 1497 | ATGATTTCCAGAGTTTTTGAGCT | 1400 |
| | | GAGAUCA | | AGAAGGAGATCAAATTACATTT | |
| NM_005259 | 1483 | UUGAGCUAGAAGG | 1498 | TGATTTCCAGAGTTTTTGAGCTA | 1401 |
| | | AGAUCAA | | GAAGGAGATCAAATTACATTTA | |
| NM_005259 | 1495 | GAGAUCAAAUUAC | 1499 | TTTTTGAGCTAGAAGGAGATCAA | 1402 |
| | | AUUUAUG | | ATTACATTTATGTTCCTATATA | |
| NM_005259 | 1498 | AUCAAAUUACAUU | 1500 | TTGAGCTAGAAGGAGATCAAATT | 1403 |
| | | UAUGUUC | | ACATTTATGTTCCTATATATTA | |
| NM_005259 | 1500 | CAAAUUACAUUUA | 1501 | GAGCTAGAAGGAGATCAAATTAC | 1404 |
| | | UGUUCCU | | ATTTATGTTCCTATATATTACA | |
| NM_005259 | 1502 | AAUUACAUUUAUG | 1502 | GCTAGAAGGAGATCAAATTACAT | 1405 |
| | | UUCCUAU | | TTATGTTCCTATATATTACAAC | |
| NM_005259 | 1516 | UCCUAUAUAUUAC | 1503 | AAATTACATTTATGTTCCTATAT | 1406 |
| | | AACAUCG | | ATTACAACATCGGCGAGGAAAT | |
| NM_005259 | 1538 | GAGGAAAUGAAAG | 1504 | TATTACAACATCGGCGAGGAAAT | 1407 |
| | | CGAUUCU | | GAAAGCGATTCTCCTTGAGTTC | |
| NM_005259 | 1568 | UGAUGAAUUAAAG | 1505 | ATTCTCCTTGAGTTCTGATGAAT | 1408 |
| | | GAGUAUG | | TAAAGGAGTATGCTTTAAAGTC | |
| NM_005259 | 1583 | GUAUGCUUUAAAG | 1506 | TGATGAATTAAAGGAGTATGCTT | 1409 |
| | | UCUAUUU | | TAAAGTCTATTTCTTTAAAGTT | |
| NM_005259 | 1589 | UUUAAAGUCUAUU | 1507 | ATTAAAGGAGTATGCTTTAAAGT | 1410 |
| | | UCUUUAA | | CTATTTCTTTAAAGTTTTGTTT | |
| NM_005259 | 1595 | GUCUAUUUCUUUA | 1508 | GGAGTATGCTTTAAAGTCTATTT | 1411 |
| | | AAGUUUU | | CTTTAAAGTTTTGTTTAATATT | |
| NM_005259 | 1663 | AUUGUUAUAUACC | 1509 | TTGGTAAAATGCAGGATTGTTAT | 1412 |
| | | AUCAUUC | | ATACCATCATTCGAATCATCCT | |
| NM_005259 | 1663 | AUUGUUAUAUACC | 1510 | TTGGTAAAATGCAGGATTGTTAT | 1413 |
| | | AUCAUUC | | ATACCATCATTCGAATCATCCT | |
| NM_005259 | 1682 | CGAAUCAUCCUUA | 1511 | TTATATACCATCATTCGAATCAT | 1414 |
| | | AACACUU | | CCTTAAACACTTGAATTTATAT | |
| NM_005259 | 1691 | CUUAAACACUUGA | 1512 | ATCATTCGAATCATCCTTAAACA | 1415 |
| | | AUUUAUA | | CTTGAATTTATATTGTATGGTA | |
| NM_005259 | 1694 | AAACACUUGAAUU | 1513 | ATTCGAATCATCCTTAAACACTT | 1416 |
| | | UAUAUUG | | GAATTTATATTGTATGGTAGTA | |
| NM_005259 | 1698 | ACUUGAAUUUAUA | 1514 | GAATCATCCTTAAACACTTGAAT | 1417 |
| | | UUGUAUG | | TTATATTGTATGGTAGTATACT | |
| NM_005259 | 1753 | AGGGAUGGUGCAG | 1515 | AAATTCCACAAAAATAGGGATGG | 1418 |
| | | CAUAUGC | | TGCAGCATATGCAATTTCCATT | |
| NM_005259 | 1761 | UGCAGCAUAUGCA | 1516 | CAAAAATAGGGATGGTGCAGCAT | 1419 |
| | | AUUUCCA | | ATGCAATTTCCATTCCTATTAT | |
| NM_005259 | 2010 | GGAGAAACUGCAU | 1517 | TTCTGTAGCATACTTGGAGAAAC | 1420 |
| | | UAUCUUA | | TGCATTATCTTAAAAGGCAGTC | |
| NM_005259 | 2080 | UUGGAGAAGUAUG | 1518 | GTCAAAATAACATACTTGGAGAA | 1421 |
| | | UAAUUUU | | GTATGTAATTTTGTCTTTGGAA | |
| NM_005259 | 2119 | ACUGCCUUUGCAA | 1519 | TTGGAAAATTACAACACTGCCTT | 1422 |
| | | CACUGCA | | TGCAACACTGCAGTTTTTATGG | |
| NM_005259 | 2124 | CUUUGCAACACUG | 1520 | AAATTACAACACTGCCTTTGCAA | 1423 |
| | | CAGUUUU | | CACTGCAGTTTTTATGGTAAAA | |
| NM_005259 | 2155 | AAUAGAAAUGAUC | 1521 | TTTTTATGGTAAAATAATAGAAA | 1424 |
| | | GACUCUA | | TGATCGACTCTATCAATATTGT | |
| NM_005259 | 2156 | AUAGAAAUGAUCG | 1522 | TTTTATGGTAAAATAATAGAAAT | 1425 |
| | | ACUCUAU | | GATCGACTCTATCAATATTGTA | |
| NM_005259 | 2164 | GAUCGACUCUAUC | 1523 | TAAAATAATAGAAATGATCGACT | 1426 |
| | | AAUAUUG | | CTATCAATATTGTATAAAAAGA | |
| NM_005259 | 2168 | GACUCUAUCAAUA | 1524 | ATAATAGAAATGATCGACTCTAT | 1427 |
| | | UUGUAUA | | CAATATTGTATAAAAAGACTGA | |
| NM_005259 | 2188 | AAAAGACUGAAAC | 1525 | TATCAATATTGTATAAAAAGACT | 1428 |
| | | AAUGCAU | | GAAACAATGCATTTATATAATA | |
| NM_005259 | 2193 | ACUGAAACAAUGC | 1526 | ATATTGTATAAAAAGACTGAAAC | 1429 |
| | | AUUUAUA | | AATGCATTTATATAATATGTAT | |
| NM_005259 | 2195 | UGAAACAAUGCAU | 1527 | ATTGTATAAAAAGACTGAAACAA | 1430 |
| | | UUAUAUA | | TGCATTTATATAATATGTATAC | |
| NM_005259 | 2196 | GAAACAAUGCAUU | 1528 | TTGTATAAAAAGACTGAAACAAT | 1431 |
| | | UAUAUAA | | GCATTTATATAATATGTATACA | |
| NM_005259 | 2209 | UAUAUAAUAUGUA | 1529 | CTGAAACAATGCATTTATATAAT | 1432 |
| | | UACAAUA | | ATGTATACAATATTGTTTTGTA | |
| NM_005259 | 2212 | AUAAUAUGUAUAC | 1530 | AAACAATGCATTTATATAATATG | 1433 |
| | | AAUAUUG | | TATACAATATTGTTTTGTAAAT | |
| NM_005259 | 2216 | UAUGUAUACAAUA | 1531 | AATGCATTTATATAATATGTATA | 1434 |
| | | UUGUUUU | | CAATATTGTTTTGTAAATAAGT | |
| NM_005259 | 2230 | UGUUUUGUAAAUA | 1532 | ATATGTATACAATATTGTTTTGT | 1435 |
| | | AGUGUCU | | AAATAAGTGTCTCCTTTTTTAT | |
| NM_005259 | 2232 | UUUUGUAAAUAAG | 1533 | ATGTATACAATATTGTTTTGTAA | 1436 |
| | | UGUCUCC | | ATAAGTGTCTCCTTTTTTATTT | |
| NM_005259 | 2233 | UUUGUAAAUAAGU | 1534 | TGTATACAATATTGTTTTGTAAA | 1437 |
| | | GUCUCCU | | TAAGTGTCTCCTTTTTTATTTA | |
| NM_005259 | 2234 | UUGUAAAUAAGUG | 1535 | GTATACAATATTGTTTTGTAAAT | 1438 |
| | | UCUCCUU | | AAGTGTCTCCTTTTTTATTTAC | |
| NM_005259 | 2274 | UUUUACACUAAGG | 1536 | TTTACTTTGGTATATTTTTACAC | 1439 |
| | | ACAUUUC | | TAAGGACATTTCAAATTAAGTA | |
| NM_005259 | 2276 | UUACACUAAGGAC | 1537 | TACTTTGGTATATTTTTACACTA | 1440 |
| | | AUUUCAA | | AGGACATTTCAAATTAAGTACT | |
| NM_005259 | 2283 | AAGGACAUUUCAA | 1538 | GTATATTTTTACACTAAGGACAT | 1441 |
| | | AUUAAGU | | TTCAAATTAAGTACTAAGGCAC | |
| NM_005259 | 2363 | AUUAGCAGAUUAA | 1539 | TATATTTCAGAGCAAATTAGCAG | 1442 |
| | | AUAGUGG | | ATTAAATAGTGGTCTTAAAACT | |
| NM_005259 | 2364 | UUAGCAGAUUAAA | 1540 | ATATTTCAGAGCAAATTAGCAGA | 1443 |
| | | UAGUGGU | | TTAAATAGTGGTCTTAAAACTC | |
| NM_005259 | 2365 | UAGCAGAUUAAAU | 1541 | TATTTCAGAGCAAATTAGCAGAT | 1444 |
| | | AGUGGUC | | TAAATAGTGGTCTTAAAACTCC | |
| NM_005259 | 2397 | AUGUUAAUGAUUA | 1542 | GTCTTAAAACTCCATATGTTAAT | 1445 |
| | | GAUGGUU | | GATTAGATGGTTATATTACAAT | |
| NM_005259 | 2406 | AUUAGAUGGUUAU | 1543 | CTCCATATGTTAATGATTAGATG | 1446 |
| | | AUUACAA | | GTTATATTACAATCATTTTATA | |
| NM_005259 | 2411 | AUGGUUAUAUUAC | 1544 | TATGTTAATGATTAGATGGTTAT | 1447 |
| | | AAUCAUU | | ATTACAATCATTTTATATTTTT | |
| NM_005259 | 2439 | UUUUACAUGAUUA | 1545 | AATCATTTTATATTTTTTTACAT | 1448 |
| | | ACAUUCA | | GATTAACATTCACTTATGGATT | |
| NM_005259 | 2440 | UUUACAUGAUUAA | 1546 | ATCATTTTATATTTTTTTACATG | 1449 |
| | | CAUUCAC | | ATTAACATTCACTTATGGATTC | |
| NM_005259 | 2475 | GGCUGUAUAAAGU | 1547 | TTATGGATTCATGATGGCTGTAT | 1450 |
| | | GAAUUUG | | AAAGTGAATTTGAAATTTCAAT | |
| NM_005259 | 2491 | UUUGAAAUUUCAA | 1548 | GCTGTATAAAGTGAATTTGAAAT | 1451 |
| | | UGGUUUA | | TTCAATGGTTTACTGTCATTGT | |
| NM_005259 | 2493 | UGAAAUUUCAAUG | 1549 | TGTATAAAGTGAATTTGAAATTT | 1452 |
| | | GUUUACU | | CAATGGTTTACTGTCATTGTGT | |
| NM_005259 | 2513 | GUCAUUGUGUUUA | 1550 | TTTCAATGGTTTACTGTCATTGT | 1453 |
| | | AAUCUCA | | GTTTAAATCTCAACGTTCCATT | |
| NM_005259 | 2519 | GUGUUUAAAUCUC | 1551 | TGGTTTACTGTCATTGTGTTTAA | 1454 |
| | | AACGUUC | | ATCTCAACGTTCCATTATTTTA | |
| NM_005259 | 2521 | GUUUAAAUCUCAA | 1552 | GTTTACTGTCATTGTGTTTAAAT | 1455 |
| | | CGUUCCA | | CTCAACGTTCCATTATTTTAAT | |
| NM_005259 | 2609 | AAUAAACUGAUGC | 1553 | TTATCTGAAATGAAGAATAAACT | 1456 |
| | | UAUCUCA | | GATGCTATCTCAACAATAACTG | |
| NM_005259 | 2622 | UAUCUCAACAAUA | 1554 | AGAATAAACTGATGCTATCTCAA | 1457 |
| | | ACUGUUA | | CAATAACTGTTACTTTTATTTT | |
| NM_005259 | 2623 | AUCUCAACAAUAA | 1555 | GAATAAACTGATGCTATCTCAAC | 1458 |
| | | CUGUUAC | | AATAACTGTTACTTTTATTTTA | |
| NM_005259 | 2649 | UUUAUAAUUUGAU | 1556 | AACTGTTACTTTTATTTTATAAT | 1459 |
| | | AAUGAAU | | TTGATAATGAATATATTTCTGC | |
| NM_005259 | 2657 | UUGAUAAUGAAUA | 1557 | CTTTTATTTTATAATTTGATAAT | 1460 |
| | | UAUUUCU | | GAATATATTTCTGCATTTATTT | |
| NM_005259 | 2658 | UGAUAAUGAAUAU | 1558 | TTTTATTTTATAATTTGATAATG | 1461 |
| | | AUUUCUG | | AATATATTTCTGCATTTATTTA | |
| NM_005259 | 2661 | UAAUGAAUAUAUU | 1559 | TATTTTATAATTTGATAATGAAT | 1462 |
| | | UCUGCAU | | ATATTTCTGCATTTATTTACTT | |
| NM_005259 | 2671 | AUUUCUGCAUUUA | 1560 | TTTGATAATGAATATATTTCTGC | 1463 |
| | | UUUACUU | | ATTTATTTACTTCTGTTTTGTA | |
| NM_005259 | 2675 | CUGCAUUUAUUUA | 1561 | ATAATGAATATATTTCTGCATTT | 1464 |
| | | CUUCUGU | | ATTTACTTCTGTTTTGTAAATT | |
| NM_005259 | 2711 | UUGUUAAUCAAAU | 1562 | TTGTAAATTGGGATTTTGTTAAT | 1465 |
| | | UUAUUGU | | CAAATTTATTGTACTATGACTA | |
| NM_005259 | 2733 | UAUGACUAAAUGA | 1563 | TCAAATTTATTGTACTATGACTA | 1466 |
| | | AAUUAUU | | AATGAAATTATTTCTTACATCT | |
| NM_005259 | 2734 | AUGACUAAAUGAA | 1564 | CAAATTTATTGTACTATGACTAA | 1467 |
| | | AUUAUUU | | ATGAAATTATTTCTTACATCTA | |
| NM_005259 | 2750 | AUUUCUUACAUCU | 1565 | TGACTAAATGAAATTATTTCTTA | 1468 |
| | | AAUUUGU | | CATCTAATTTGTAGAAACAGTA | |
| NM_005259 | 2753 | UCUUACAUCUAAU | 1566 | CTAAATGAAATTATTTCTTACAT | 1469 |
| | | UUGUAGA | | CTAATTTGTAGAAACAGTATAA | |
| NM_005259 | 2765 | UUUGUAGAAACAG | 1567 | ATTTCTTACATCTAATTTGTAGA | 1470 |
| | | UAUAAGU | | AACAGTATAAGTTATATTAAAG | |
| NM_005259 | 2767 | UGUAGAAACAGUA | 1568 | TTCTTACATCTAATTTGTAGAAA | 1471 |
| | | UAAGUUA | | CAGTATAAGTTATATTAAAGTG | |
| NM_005259 | 2770 | AGAAACAGUAUAA | 1569 | TTACATCTAATTTGTAGAAACAG | 1472 |
| | | GUUAUAU | | TATAAGTTATATTAAAGTGTTT | |

TABLE 12
Mouse Mstn target gene regions sequences and target sequences

| Accession | | | SEQ | | SEQ |
|---|---|---|---|---|---|
| number | Position | Target sequence | ID NO | Gene region | ID NO |
| NM_010834 | 78 | AAGAAGAAAUAAGAAC | 1288 | TTACTCAAAAGCAAAAAGAAGAA | 1200 |
| | | AAGG | | ATAAGAACAAGGGAAAAAAAAA | |
| NM_010834 | 242 | UGUGUAAUGCAUGUGC | 1289 | TGGAAAAAGAGGGGCTGTGTAAT | 1201 |
| | | GUGG | | GCATGTGCGTGGAGACAAAACA | |
| NM_010834 | 277 | UACUCCAGAAUAGAAG | 1290 | AGACAAAACACGAGGTACTCCAG | 1202 |
| | | CCAU | | AATAGAAGCCATAAAAATTCAA | |
| NM_010834 | 523 | GAUGGCAAGCCCAAAU | 1291 | TTTCTAATGCAAGCGGATGGCAA | 1203 |
| | | GUUG | | GCCCAAATGTTGCTTTTTTAAA | |
| NM_010834 | 524 | AUGGCAAGCCCAAAUG | 1292 | TTCTAATGCAAGCGGATGGCAAG | 1204 |
| | | UUGC | | CCCAAATGTTGCTTTTTTAAAT | |
| NM_010834 | 618 | CAAGACUCCUACAACA | 1293 | ATATCTCAGACCCGTCAAGACTC | 1205 |
| | | GUGU | | CTACAACAGTGTTTGTGCAAAT | |
| NM_010834 | 780 | ACAGCCUGAAUCCAAC | 1294 | GCAAAATTGGCTCAAACAGCCTG | 1206 |
| | | UUAG | | AATCCAACTTAGGCATTGAAAT | |
| NM_010834 | 1007 | UUGGAUGGGACUGGAU | 1295 | TCGATTTTGAAGCCTTTGGATGG | 1207 |
| | | UAUC | | GACTGGATTATCGCACCCAAAA | |
| NM_010834 | 1051 | UACUGCUCAGGAGAGU | 1296 | AGATATAAGGCCAATTACTGCTC | 1208 |
| | | GUGA | | AGGAGAGTGTGAATTTGTGTTT | |
| NM_010834 | 1057 | UCAGGAGAGUGUGAAU | 1297 | AAGGCCAATTACTGCTCAGGAGA | 1209 |
| | | UUGU | | GTGTGAATTTGTGTTTTTACAA | |
| NM_010834 | 1058 | CAGGAGAGUGUGAAUU | 1298 | AGGCCAATTACTGCTCAGGAGAG | 1210 |
| | | UGUG | | TGTGAATTTGTGTTTTTACAAA | |
| NM_010834 | 1168 | CCCAUUAAUAUGCUAU | 1299 | CCGACAAAAATGTCTCCCATTAA | 1211 |
| | | AUUU | | TATGCTATATTTTAATGGCAAA | |
| NM_010834 | 1189 | AAUGGCAAAGAACAAA | 1300 | AATATGCTATATTTTAATGGCAA | 1212 |
| | | UAAU | | AGAACAAATAATATATGGGAAA | |
| NM_010834 | 1190 | AUGGCAAAGAACAAAU | 1301 | ATATGCTATATTTTAATGGCAAA | 1213 |
| | | AAUA | | GAACAAATAATATATGGGAAAA | |
| NM_010834 | 1192 | GGCAAAGAACAAAUAA | 1302 | ATGCTATATTTTAATGGCAAAGA | 1214 |
| | | UAUA | | ACAAATAATATATGGGAAAATT | |
| NM_010834 | 1262 | UGCAUUAGGUUAGAAA | 1303 | GGTGCTCATGAGCTTTGCATTAG | 1215 |
| | | UUUC | | GTTAGAAATTTCCCAAGTCATG | |
| NM_010834 | 1325 | UCAAGCACCACAGGCU | 1304 | TTCGAAACTGTGAATTCAAGCAC | 1216 |
| | | GUAG | | CACAGGCTGTAGGCCTTGAGTA | |
| NM_010834 | 1370 | AGCACAAGCUACAGUG | 1305 | TGCTCTAGTAACGTAAGCACAAG | 1217 |
| | | UAUG | | CTACAGTGTATGAACTAAAAGA | |
| NM_010834 | 1416 | GUUGGCAUUCAACCAC | 1306 | AGAATAGATGCAATGGTTGGCAT | 1218 |
| | | CAAA | | TCAACCACCAAAATAAACCATA | |
| NM_010834 | 1449 | UAGGAUGUUGUAUGAU | 1307 | AAATAAACCATACTATAGGATGT | 1219 |
| | | UUCC | | TGTATGATTTCCAGAGTTTTTG | |
| NM_010834 | 1454 | UGUUGUAUGAUUUCCA | 1308 | AACCATACTATAGGATGTTGTAT | 1220 |
| | | GAGU | | GATTTCCAGAGTTTTTGAAATA | |
| NM_010834 | 1485 | AUGGAGAUCAAAUUAC | 1309 | GAGTTTTTGAAATAGATGGAGAT | 1221 |
| | | AUUU | | CAAATTACATTTATGTCCATAT | |
| NM_010834 | 1489 | AGAUCAAAUUACAUUU | 1310 | TTTTGAAATAGATGGAGATCAAA | 1222 |
| | | AUGU | | TTACATTTATGTCCATATATGT | |
| NM_010834 | 1600 | AGUCUUAUUUCCUAAC | 1311 | ATGATTAAAAGGTAAAGTCTTAT | 1223 |
| | | AGUU | | TTCCTAACAGTTTCACTTAATA | |
| NM_010834 | 1602 | UCUUAUUUCCUAACAG | 1312 | GATTAAAAGGTAAAGTCTTATTT | 1224 |
| | | UUUC | | CCTAACAGTTTCACTTAATATT | |
| NM_010834 | 1603 | CUUAUUUCCUAACAGU | 1313 | ATTAAAAGGTAAAGTCTTATTTC | 1225 |
| | | UUCA | | CTAACAGTTTCACTTAATATTT | |
| NM_010834 | 1632 | UACGGAAGAAUCUAUA | 1314 | GTTTCACTTAATATTTACGGAAG | 1226 |
| | | UGUA | | AATCTATATGTAGCCTTTGTAA | |
| NM_010834 | 1633 | ACGGAAGAAUCUAUAU | 1315 | TTTCACTTAATATTTACGGAAGA | 1227 |
| | | GUAG | | ATCTATATGTAGCCTTTGTAAA | |
| NM_010834 | 1635 | GGAAGAAUCUAUAUGU | 1316 | TCACTTAATATTTACGGAAGAAT | 1228 |
| | | AGCC | | CTATATGTAGCCTTTGTAAAGT | |
| NM_010834 | 1656 | UUGUAAAGUGUAGGAU | 1317 | ATCTATATGTAGCCTTTGTAAAG | 1229 |
| | | UGUU | | TGTAGGATTGTTATCATTTAAA | |
| NM_010834 | 1657 | UGUAAAGUGUAGGAUU | 1318 | TCTATATGTAGCCTTTGTAAAGT | 1230 |
| | | GUUA | | GTAGGATTGTTATCATTTAAAA | |
| NM_010834 | 1691 | CAUGUACACUUAUAUU | 1319 | ATCATTTAAAAACATCATGTACA | 1231 |
| | | UGUA | | CTTATATTTGTATTGTATACTT | |
| NM_010834 | 1696 | ACACUUAUAUUUGUAU | 1320 | TTAAAAACATCATGTACACTTAT | 1232 |
| | | UGUA | | ATTTGTATTGTATACTTGGTAA | |
| NM_010834 | 1704 | AUUUGUAUUGUAUACU | 1321 | ATCATGTACACTTATATTTGTAT | 1233 |
| | | UGGU | | TGTATACTTGGTAAGATAAAAT | |
| NM_010834 | 1705 | UUUGUAUUGUAUACUU | 1322 | TCATGTACACTTATATTTGTATT | 1234 |
| | | GGUA | | GTATACTTGGTAAGATAAAATT | |
| NM_010834 | 1733 | UUCCACAAAGUAGGAA | 1323 | CTTGGTAAGATAAAATTCCACAA | 1235 |
| | | UGGG | | AGTAGGAATGGGGCCTTACATA | |
| NM_010834 | 1928 | UUAAGGAAAGAAGAAU | 1324 | TTCAATGCATTTTCTTTAAGGAA | 1236 |
| | | CUUU | | AGAAGAATCTTTTTTTCTAGAG | |
| NM_010834 | 2008 | AAGGCAGCCAAACAGU | 1325 | ACTGCTGCATCTTAAAAGGCAGC | 1237 |
| | | AUUC | | CAAACAGTATTCATTTTTTAAT | |
| NM_010834 | 2057 | UCUGCCUUUAUCACAU | 1326 | ATTTCAAAATCACTGTCTGCCTT | 1238 |
| | | GGCA | | TATCACATGGCAATTTTGTGGT | |
| NM_010834 | 2093 | AUGGAAAUGACUGGUU | 1327 | TTTTGTGGTAAAATAATGGAAAT | 1239 |
| | | CUAU | | GACTGGTTCTATCAATATTGTA | |
| NM_010834 | 2126 | AAGACUCUGAAACAAU | 1328 | ATCAATATTGTATAAAAGACTCT | 1240 |
| | | UACA | | GAAACAATTACATTTATATAAT | |
| NM_010834 | 2131 | UCUGAAACAAUUACAU | 1329 | TATTGTATAAAAGACTCTGAAAC | 1241 |
| | | UUAU | | AATTACATTTATATAATATGTA | |
| NM_010834 | 2132 | CUGAAACAAUUACAUU | 1330 | ATTGTATAAAAGACTCTGAAACA | 1242 |
| | | UAUA | | ATTACATTTATATAATATGTAT | |
| NM_010834 | 2133 | UGAAACAAUUACAUUU | 1331 | TTGTATAAAAGACTCTGAAACAA | 1243 |
| | | AUAU | | TTACATTTATATAATATGTATA | |
| NM_010834 | 2143 | ACAUUUAUAUAAUAUG | 1332 | GACTCTGAAACAATTACATTTAT | 1244 |
| | | UAUA | | ATAATATGTATACAATATTGTT | |
| NM_010834 | 2151 | AUAAUAUGUAUACAAU | 1333 | AACAATTACATTTATATAATATG | 1245 |
| | | AUUG | | TATACAATATTGTTTTGTAAAT | |
| NM_010834 | 2152 | UAAUAUGUAUACAAUA | 1334 | ACAATTACATTTATATAATATGT | 1246 |
| | | UUGU | | ATACAATATTGTTTTGTAAATA | |
| NM_010834 | 2155 | UAUGUAUACAAUAUUG | 1335 | ATTACATTTATATAATATGTATA | 1247 |
| | | UUUU | | CAATATTGTTTTGTAAATAAGT | |
| NM_010834 | 2169 | UGUUUUGUAAAUAAGU | 1336 | ATATGTATACAATATTGTTTTGT | 1248 |
| | | GUCU | | AAATAAGTGTCTCCTTTTATAT | |
| NM_010834 | 2171 | UUUUGUAAAUAAGUGU | 1337 | ATGTATACAATATTGTTTTGTAA | 1249 |
| | | CUCC | | ATAAGTGTCTCCTTTTATATTT | |
| NM_010834 | 2172 | UUUGUAAAUAAGUGUC | 1338 | TGTATACAATATTGTTTTGTAAA | 1250 |
| | | UCCU | | TAAGTGTCTCCTTTTATATTTA | |
| NM_010834 | 2173 | UUGUAAAUAAGUGUCU | 1339 | GTATACAATATTGTTTTGTAAAT | 1251 |
| | | CCUU | | AAGTGTCTCCTTTTATATTTAC | |
| NM_010834 | 2175 | GUAAAUAAGUGUCUCC | 1340 | ATACAATATTGTTTTGTAAATAA | 1252 |
| | | UUUU | | GTGTCTCCTTTTATATTTACTT | |
| NM_010834 | 2213 | UUUUACACUAAUGAAA | 1341 | TTTACTTTGGTATATTTTTACAC | 1253 |
| | | UUUC | | TAATGAAATTTCAAATCATTAA | |
| NM_010834 | 2214 | UUUACACUAAUGAAAU | 1342 | TTACTTTGGTATATTTTTACACT | 1254 |
| | | UUCA | | AATGAAATTTCAAATCATTAAA | |
| NM_010834 | 2215 | UUACACUAAUGAAAUU | 1343 | TACTTTGGTATATTTTTACACTA | 1255 |
| | | UCAA | | ATGAAATTTCAAATCATTAAAG | |
| NM_010834 | 2222 | AAUGAAAUUUCAAAUC | 1344 | GTATATTTTTACACTAATGAAAT | 1256 |
| | | AUUA | | TTCAAATCATTAAAGTACAAAG | |
| NM_010834 | 2232 | CAAAUCAUUAAAGUAC | 1345 | ACACTAATGAAATTTCAAATCAT | 1257 |
| | | AAAG | | TAAAGTACAAAGACATGTCATG | |
| NM_010834 | 2299 | AUUAGCAGAUUCAAUA | 1346 | TCTATTTCAGAGTGAATTAGCAG | 1258 |
| | | GUGG | | ATTCAATAGTGGTCTTAAAACT | |
| NM_010834 | 2301 | UAGCAGAUUCAAUAGU | 1347 | TATTTCAGAGTGAATTAGCAGAT | 1259 |
| | | GGUC | | TCAATAGTGGTCTTAAAACTCT | |
| NM_010834 | 2332 | UAUGUUAAGAUUAGAA | 1348 | GGTCTTAAAACTCTGTATGTTAA | 1260 |
| | | GGUU | | GATTAGAAGGTTATATTACAAT | |
| NM_010834 | 2335 | GUUAAGAUUAGAAGGU | 1349 | CTTAAAACTCTGTATGTTAAGAT | 1261 |
| | | UAUA | | TAGAAGGTTATATTACAATCAA | |
| NM_010834 | 2337 | UAAGAUUAGAAGGUUA | 1350 | TAAAACTCTGTATGTTAAGATTA | 1262 |
| | | UAUU | | GAAGGTTATATTACAATCAATT | |
| NM_010834 | 2346 | AAGGUUAUAUUACAAU | 1351 | GTATGTTAAGATTAGAAGGTTAT | 1263 |
| | | CAAU | | ATTACAATCAATTTATGTATTT | |
| NM_010834 | 2348 | GGUUAUAUUACAAUCA | 1352 | ATGTTAAGATTAGAAGGTTATAT | 1264 |
| | | AUUU | | TACAATCAATTTATGTATTTTT | |
| NM_010834 | 2350 | UUAUAUUACAAUCAAU | 1353 | GTTAAGATTAGAAGGTTATATTA | 1265 |
| | | UUAU | | CAATCAATTTATGTATTTTTTA | |
| NM_010834 | 2353 | UAUUACAAUCAAUUUA | 1354 | AAGATTAGAAGGTTATATTACAA | 1266 |
| | | UGUA | | TCAATTTATGTATTTTTTACAT | |
| NM_010834 | 2354 | AUUACAAUCAAUUUAU | 1355 | AGATTAGAAGGTTATATTACAAT | 1267 |
| | | GUAU | | CAATTTATGTATTTTTTACATT | |
| NM_010834 | 2376 | UUUACAUUAUCAACAU | 1356 | TCAATTTATGTATTTTTTACATT | 1268 |
| | | UCAC | | ATCAACATTCACTTATGGTTTC | |
| NM_010834 | 2478 | UACUAAACAUACCAAC | 1357 | AAAAATTACAAGCATTACTAAAC | 1269 |
| | | AUGU | | ATACCAACATGTATCTAAAGAA | |
| NM_010834 | 2480 | CUAAACAUACCAACAU | 1358 | AAATTACAAGCATTACTAAACAT | 1270 |
| | | GUAU | | ACCAACATGTATCTAAAGAAAT | |
| NM_010834 | 2488 | ACCAACAUGUAUCUAA | 1359 | AGCATTACTAAACATACCAACAT | 1271 |
| | | AGAA | | GTATCTAAAGAAATACAAATAT | |
| NM_010834 | 2507 | AAUACAAAUAUGGUAU | 1360 | ACATGTATCTAAAGAAATACAAA | 1272 |
| | | CUCA | | TATGGTATCTCAATAACAGCTA | |
| NM_010834 | 2515 | UAUGGUAUCUCAAUAA | 1361 | CTAAAGAAATACAAATATGGTAT | 1273 |
| | | CAGC | | CTCAATAACAGCTACTTTTTTA | |
| NM_010834 | 2520 | UAUCUCAAUAACAGCU | 1362 | GAAATACAAATATGGTATCTCAA | 1274 |
| | | ACUU | | TAACAGCTACTTTTTTATTTTA | |
| NM_010834 | 2521 | AUCUCAAUAACAGCUA | 1363 | AAATACAAATATGGTATCTCAAT | 1275 |
| | | CUUU | | AACAGCTACTTTTTTATTTTAT | |
| NM_010834 | 2522 | UCUCAAUAACAGCUAC | 1364 | AATACAAATATGGTATCTCAATA | 1276 |
| | | UUUU | | ACAGCTACTTTTTTATTTTATA | |
| NM_010834 | 2546 | UUUAUAAUUUGACAAU | 1365 | CAGCTACTTTTTTATTTTATAAT | 1277 |
| | | GAAU | | TTGACAATGAATACATTTCTTT | |
| NM_010834 | 2553 | UUUGACAAUGAAUACA | 1366 | TTTTTTATTTTATAATTTGACAA | 1278 |
| | | UUUC | | TGAATACATTTCTTTTATTTAC | |
| NM_010834 | 2554 | UUGACAAUGAAUACAU | 1367 | TTTTTATTTTATAATTTGACAAT | 1279 |
| | | UUCU | | GAATACATTTCTTTTATTTACT | |
| NM_010834 | 2555 | UGACAAUGAAUACAUU | 1368 | TTTTATTTTATAATTTGACAATG | 1280 |
| | | UCUU | | AATACATTTCTTTTATTTACTT | |
| NM_010834 | 2588 | UUUUAUAAAUUGGAAC | 1369 | TTTTATTTACTTCAGTTTTATAA | 1281 |
| | | UUUG | | ATTGGAACTTTGTTTATCAAAT | |
| NM_010834 | 2589 | UUUAUAAAUUGGAACU | 1370 | TTTATTTACTTCAGTTTTATAAA | 1282 |
| | | UUGU | | TTGGAACTTTGTTTATCAAATG | |
| NM_010834 | 2590 | UUAUAAAUUGGAACUU | 1371 | TTATTTACTTCAGTTTTATAAAT | 1283 |
| | | UGUU | | TGGAACTTTGTTTATCAAATGT | |
| NM_010834 | 2598 | UGGAACUUUGUUUAUC | 1372 | TTCAGTTTTATAAATTGGAACTT | 1284 |
| | | AAAU | | TGTTTATCAAATGTATTGTACT | |
| NM_010834 | 2623 | GUACUCAUAGCUAAAU | 1373 | TTTATCAAATGTATTGTACTCAT | 1285 |
| | | GAAA | | AGCTAAATGAAATTATTTCTTA | |
| NM_010834 | 2629 | AUAGCUAAAUGAAAUU | 1374 | AAATGTATTGTACTCATAGCTAA | 1286 |
| | | AUUU | | ATGAAATTATTTCTTACATAAA | |
| NM_010834 | 2631 | AGCUAAAUGAAAUUAU | 1375 | ATGTATTGTACTCATAGCTAAAT | 1287 |
| | | UUCU | | GAAATTATTTCTTACATAAAAA | |

In some embodiments, a target region of a human MSTN specific siRNA comprises the nucleic acid sequence of any one of SEQ ID NOs: 1376-1472. In some embodiments, a target region of a mouse Mstn specific siRNA comprises the nucleic acid sequence of any one of SEQ ID NOs: 1200-1287.

In some embodiments, a human MSTN specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides to a human MSTN target nucleic acid sequence as set forth in SEQ ID NOs: 1376-1472. In some embodiments, a human MSTN specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides of to a human MSTN target nucleic acid sequence as set forth in SEQ ID NOs: 1376-1472, except for at least 1, at least 2, at least 3, at least 4, or at least 5 mismatch within the region of complementarity to the nucleotide sequence of SEQ ID NOs: 1376-1472.

In some embodiments, a mouse Mstn specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides to a mouse Mstn target nucleic acid sequence as set forth in SEQ ID NOs: 1200-1287. In some embodiments, a mouse Mstn specific siRNA described herein comprises a region of complementarity to at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, or more consecutive nucleotides of to a mouse Mstn target nucleic acid sequence as set forth in SEQ ID NOs: 1200-1287, except for at least 1, at least 2, at least 3, at least 4, or at least 5 mismatch within the region of complementarity to the nucleotide sequence of SEQ ID NOs: 1200-1287.

In some embodiments, a human MSTN specific siRNA may be at least 80%, at least 85%, at least 90%, at least 91%, at least 92%, at least 93%, at least 94%, at least 95%, at least 96%, at least 97%, at least 98%, at least 99% or 100% complementary to any one of SEQ ID NOs: 1473-1569. In some embodiments, a complementary nucleotide sequence need not be 100% complementary to that of a human MSTN target sequence to be specifically hybridizable or specific for an MSTN. In some embodiments, the antisense strand of a human MSTN specific siRNA is fully complementarity with all or a portion of its target sequence. In some embodiments, the antisense strand of a human MSTN specific siRNA includes 1, 2, 3, or more mismatches to the target sequence. In some embodiments, a human MSTN specific siRNA comprises an antisense strand comprising a nucleotide sequence that is complementary (e.g., at least 85%, at least 90%, at least 95%, at least 96%, at least 97%, at least 98%, at least 99%, or 100%) to a target RNA sequence as set forth in any one of SEQ ID NOs: 1473-1569. In some embodiments, a human MSTN specific siRNA comprises an antisense strand comprising a nucleic acid sequence that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in any one of SEQ ID NOs: 1473-1569. In some embodiments, a human MSTN specific siRNA comprises an antisense strand comprising a nucleic acid sequence that is complementary the sequence as set forth in any one of SEQ ID NOs: 1473-1569.

In some embodiments, a mouse Mstn specific siRNA may be at least 80%, at least 85%, at least 90%, at least 91%, at least 92%, at least 93%, at least 94%, at least 95%, at least 96%, at least 97%, at least 98%, at least 99% or 100% complementary to any one of SEQ ID NOs: 1288-1375. In some embodiments, a complementary nucleotide sequence need not be 100% complementary to that of a mouse Mstn target sequence to be specifically hybridizable or specific for a mouse Mstn. In some embodiments, the antisense strand of a mouse Mstn specific siRNA is fully complementarity with all or a portion of its target sequence. In some embodiments, the antisense strand of a mouse Mstn specific siRNA includes 1, 2, 3, or more mismatches to the target sequence. In some embodiments, a mouse Mstn specific siRNA comprises an antisense strand comprising a nucleotide sequence that is complementary (e.g., at least 85%, at least 90%, at least 95%, at least 96%, at least 97%, at least 98%, at least 99%, or 100%) to a target RNA sequence as set forth in any one of SEQ ID NOs: 1288-1375. In some embodiments, a mouse Mstn specific siRNA comprises an antisense strand comprising a nucleic acid sequence that is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of the sequence as set forth in any one of SEQ ID NOs: 1288-1375. In some embodiments, a mouse Mstn specific siRNA comprises an antisense strand comprising a nucleic acid sequence that is complementary the sequence as set forth in any one of SEQ ID NOs: 1288-1375.

Exemplary MSTN specific siRNA sequences (both antisense and sense strand) are set forth in Tables 13 and 14.

TABLE 13
Exemplary mouse Mstn specific siRNA sequences

| Accession | | | SEQ | | SEQ |
|---|---|---|---|---|---|
| number | Position | Sense Strand | ID NO | Antisense Strand | ID NO |
| NM_010834 | 78 | AGAAAUAAGAACAAGA | 1658 | UCUUGUUCUUAUUUCUUCUUU | 1570 |
| NM_010834 | 242 | UAAUGCAUGUGCGUGA | 1659 | UCACGCACAUGCAUUACACUU | 1571 |
| NM_010834 | 277 | CCAGAAUAGAAGCCAA | 1660 | UUGGCUUCUAUUCUGGAGUUU | 1572 |
| NM_010834 | 523 | GCAAGCCCAAAUGUUA | 1661 | UAACAUUUGGGCUUGCCAUUU | 1573 |
| NM_010834 | 524 | CAAGCCCAAAUGUUGA | 1662 | UCAACAUUUGGGCUUGCCAUU | 1574 |
| NM_010834 | 618 | ACUCCUACAACAGUGA | 1663 | UCACUGUUGUAGGAGUCUUUU | 1575 |
| NM_010834 | 780 | CCUGAAUCCAACUUAA | 1664 | UUAAGUUGGAUUCAGGCUGUU | 1576 |
| NM_010834 | 1007 | AUGGGACUGGAUUAUA | 1665 | UAUAAUCCAGUCCCAUCCAUU | 1577 |
| NM_010834 | 1051 | GCUCAGGAGAGUGUGA | 1666 | UCACACUCUCCUGAGCAGUUU | 1578 |
| NM_010834 | 1057 | GAGAGUGUGAAUUUGA | 1667 | UCAAAUUCACACUCUCCUGUU | 1579 |
| NM_010834 | 1058 | AGAGUGUGAAUUUGUA | 1668 | UACAAAUUCACACUCUCCUUU | 1580 |
| NM_010834 | 1168 | UUAAUAUGCUAUAUUA | 1669 | UAAUAUAGCAUAUUAAUGGUU | 1581 |
| NM_010834 | 1189 | GCAAAGAACAAAUAAA | 1670 | UUUAUUUGUUCUUUGCCAUUU | 1582 |
| NM_010834 | 1190 | CAAAGAACAAAUAAUA | 1671 | UAUUAUUUGUUCUUUGCCAUU | 1583 |
| NM_010834 | 1192 | AAGAACAAAUAAUAA | 1672 | UUAUUAUUUGUUCUUUGCCU | 1584 |
| NM_010834 | 1262 | UUAGGUUAGAAAUUUA | 1673 | UAAAUUUCUAACCUAAUGCUU | 1585 |
| NM_010834 | 1325 | GCACCACAGGCUGUAA | 1674 | UUACAGCCUGUGGUGCUUGUU | 1586 |
| NM_010834 | 1370 | CAAGCUACAGUGUAUA | 1675 | UAUACACUGUAGCUUGUGCUU | 1587 |
| NM_010834 | 1416 | GCAUUCAACCACCAAA | 1676 | UUUGGUGGUUGAAUGCCAAUU | 1588 |
| NM_010834 | 1449 | AUGUUGUAUGAUUUCA | 1677 | UGAAAUCAUACAACAUCCUUU | 1589 |
| NM_010834 | 1454 | GUAUGAUUUCCAGAGA | 1678 | UCUCUGGAAAUCAUACAACUU | 1590 |
| NM_010834 | 1485 | AGAUCAAAUUACAUUA | 1679 | UAAUGUAAUUUGAUCUCCAUU | 1591 |
| NM_010834 | 1489 | CAAAUUACAUUUAUGA | 1680 | UCAUAAAUGUAAUUUGAUCUU | 1592 |
| NM_010834 | 1600 | UUAUUUCCUAACAGUA | 1681 | UACUGUUAGGAAAUAAGACUU | 1593 |
| NM_010834 | 1602 | AUUUCCUAACAGUUUA | 1682 | UAAACUGUUAGGAAAUAAGUU | 1594 |
| NM_010834 | 1603 | UUUCCUAACAGUUUCA | 1683 | UGAAACUGUUAGGAAAUAAUU | 1595 |
| NM_010834 | 1632 | GAAGAAUCUAUAUGUA | 1684 | UACAUAUAGAUUCUUCCGUUU | 1596 |
| NM_010834 | 1633 | AAGAAUCUAUAUGUAA | 1685 | UUACAUAUAGAUUCUUCCGUU | 1597 |
| NM_010834 | 1635 | GAAUCUAUAUGUAGCA | 1686 | UGCUACAUAUAGAUUCUUCUU | 1598 |
| NM_010834 | 1656 | AAAGUGUAGGAUUGUA | 1687 | UACAAUCCUACACUUUACAUU | 1599 |
| NM_010834 | 1657 | AAGUGUAGGAUUGUUA | 1688 | UAACAAUCCUACACUUUACUU | 1600 |
| NM_010834 | 1691 | UACACUUAUAUUUGUA | 1689 | UACAAAUAUAAGUGUACAUUU | 1601 |
| NM_010834 | 1696 | UUAUAUUUGUAUUGUA | 1690 | UACAAUACAAAUAUAAGUGUU | 1602 |
| NM_010834 | 1704 | GUAUUGUAUACUUGGA | 1691 | UCCAAGUAUACAAUACAAAUU | 1603 |
| NM_010834 | 1705 | UAUUGUAUACUUGGUA | 1692 | UACCAAGUAUACAAUACAAUU | 1604 |
| NM_010834 | 1733 | ACAAAGUAGGAAUGGA | 1693 | UCCAUUCCUACUUUGUGGAUU | 1605 |
| NM_010834 | 1928 | GGAAAGAAGAAUCUUA | 1694 | UAAGAUUCUUCUUUCCUUAUU | 1606 |
| NM_010834 | 2008 | CAGCCAAACAGUAUUA | 1695 | UAAUACUGUUUGGCUGCCUUU | 1607 |
| NM_010834 | 2057 | CCUUUAUCACAUGGCA | 1696 | UGCCAUGUGAUAAAGGCAGUU | 1608 |
| NM_010834 | 2093 | AAAUGACUGGUUCUAA | 1697 | UUAGAACCAGUCAUUUCCAUU | 1609 |
| NM_010834 | 2126 | CUCUGAAACAAUUACA | 1698 | UGUAAUUGUUUCAGAGUCUUU | 1610 |
| NM_010834 | 2131 | AAACAAUUACAUUUAA | 1699 | UUAAAUGUAAUUGUUUCAGUU | 1611 |
| NM_010834 | 2132 | AACAAUUACAUUUAUA | 1700 | UAUAAAUGUAAUUGUUUCAUU | 1612 |
| NM_010834 | 2133 | ACAAUUACAUUUAUAA | 1701 | UUAUAAAUGUAAUUGUUUCUU | 1613 |
| NM_010834 | 2143 | UUAUAUAAUAUGUAUA | 1702 | UAUACAUAUUAUAUAAAUGUU | 1614 |
| NM_010834 | 2151 | UAUGUAUACAAUAUUA | 1703 | UAAUAUUGUAUACAUAUUAUU | 1615 |
| NM_010834 | 2152 | AUGUAUACAAUAUUGA | 1704 | UCAAUAUUGUAUACAUAUUUU | 1616 |
| NM_010834 | 2155 | UAUACAAUAUUGUUUA | 1705 | UAAACAAUAUUGUAUACAUUU | 1617 |
| NM_010834 | 2169 | UUGUAAAUAAGUGUCA | 1706 | UGACACUUAUUUACAAAACUU | 1618 |
| NM_010834 | 2171 | GUAAAUAAGUGUCUCA | 1707 | UGAGACACUUAUUUACAAAUU | 1619 |
| NM_010834 | 2172 | UAAAUAAGUGUCUCCA | 1708 | UGGAGACACUUAUUUACAAUU | 1620 |
| NM_010834 | 2173 | AAAUAAGUGUCUCCUA | 1709 | UAGGAGACACUUAUUUACAUU | 1621 |
| NM_010834 | 2175 | AUAAGUGUCUCCUUUA | 1710 | UAAAGGAGACACUUAUUUAUU | 1622 |
| NM_010834 | 2213 | ACACUAAUGAAAUUUA | 1711 | UAAAUUUCAUUAGUGUAAAUU | 1623 |
| NM_010834 | 2214 | CACUAAUGAAAUUUCA | 1712 | UGAAAUUUCAUUAGUGUAAUU | 1624 |
| NM_010834 | 2215 | ACUAAUGAAAUUUCAA | 1713 | UUGAAAUUUCAUUAGUGUAUU | 1625 |
| NM_010834 | 2222 | AAAUUUCAAAUCAUUA | 1714 | UAAUGAUUUGAAAUUUCAUUU | 1626 |
| NM_010834 | 2232 | UCAUUAAAGUACAAAA | 1715 | UUUUGUACUUUAAUGAUUUUU | 1627 |
| NM_010834 | 2299 | GCAGAUUCAAUAGUGA | 1716 | UCACUAUUGAAUCUGCUAAUU | 1628 |
| NM_010834 | 2301 | AGAUUCAAUAGUGGUA | 1717 | UACCACUAUUGAAUCUGCUUU | 1629 |
| NM_010834 | 2332 | UUAAGAUUAGAAGGUA | 1718 | UACCUUCUAAUCUUAACAUUU | 1630 |
| NM_010834 | 2335 | AGAUUAGAAGGUUAUA | 1719 | UAUAACCUUCUAAUCUUAAUU | 1631 |
| NM_010834 | 2337 | AUUAGAAGGUUAUAUA | 1720 | UAUAUAACCUUCUAAUCUUUU | 1632 |
| NM_010834 | 2346 | UUAUAUUACAAUCAAA | 1721 | UUUGAUUGUAAUAUAACCUUU | 1633 |
| NM_010834 | 2348 | AUAUUACAAUCAAUUA | 1722 | UAAUUGAUUGUAAUAUAACUU | 1634 |
| NM_010834 | 2350 | AUUACAAUCAAUUUAA | 1723 | UUAAAUUGAUUGUAAUAUAUU | 1635 |
| NM_010834 | 2353 | ACAAUCAAUUUAUGUA | 1724 | UACAUAAAUUGAUUGUAAUUU | 1636 |
| NM_010834 | 2354 | CAAUCAAUUUAUGUAA | 1725 | UUACAUAAAUUGAUUGUAAUU | 1637 |
| NM_010834 | 2376 | CAUUAUCAACAUUCAA | 1726 | UUGAAUGUUGAUAAUGUAAUU | 1638 |
| NM_010834 | 2478 | AAACAUACCAACAUGA | 1727 | UCAUGUUGGUAUGUUUAGUUU | 1639 |
| NM_010834 | 2480 | ACAUACCAACAUGUAA | 1728 | UUACAUGUUGGUAUGUUUAUU | 1640 |
| NM_010834 | 2488 | ACAUGUAUCUAAAGAA | 1729 | UUCUUUAGAUACAUGUUGGUU | 1641 |
| NM_010834 | 2507 | CAAAUAUGGUAUCUCA | 1730 | UGAGAUACCAUAUUUGUAUUU | 1642 |
| NM_010834 | 2515 | GUAUCUCAAUAACAGA | 1731 | UCUGUUAUUGAGAUACCAUUU | 1643 |
| NM_010834 | 2520 | UCAAUAACAGCUACUA | 1732 | UAGUAGCUGUUAUUGAGAUUU | 1644 |
| NM_010834 | 2521 | CAAUAACAGCUACUUA | 1733 | UAAGUAGCUGUUAUUGAGAUU | 1645 |
| NM_010834 | 2522 | AAUAACAGCUACUUUA | 1734 | UAAAGUAGCUGUUAUUGAGUU | 1646 |
| NM_010834 | 2546 | UAAUUUGACAAUGAAA | 1735 | UUUCAUUGUCAAAUUAUAAUU | 1647 |
| NM_010834 | 2553 | ACAAUGAAUACAUUUA | 1736 | UAAAUGUAUUCAUUGUCAAUU | 1648 |
| NM_010834 | 2554 | CAAUGAAUACAUUUCA | 1737 | UGAAAUGUAUUCAUUGUCAUU | 1649 |
| NM_010834 | 2555 | AAUGAAUACAUUUCUA | 1738 | UAGAAAUGUAUUCAUUGUCUU | 1650 |
| NM_010834 | 2588 | AUAAAUUGGAACUUUA | 1739 | UAAAGUUCCAAUUUAUAAAUU | 1651 |
| NM_010834 | 2589 | UAAAUUGGAACUUUGA | 1740 | UCAAAGUUCCAAUUUAUAAUU | 1652 |
| NM_010834 | 2590 | AAAUUGGAACUUUGUA | 1741 | UACAAAGUUCCAAUUUAUAUU | 1653 |
| NM_010834 | 2598 | ACUUUGUUUAUCAAAA | 1742 | UUUUGAUAAACAAAGUUCCUU | 1654 |
| NM_010834 | 2623 | UCAUAGCUAAAUGAAA | 1743 | UUUCAUUUAGCUAUGAGUAUU | 1655 |
| NM_010834 | 2629 | CUAAAUGAAAUUAUUA | 1744 | UAAUAAUUUCAUUUAGCUAUU | 1656 |
| NM_010834 | 2631 | AAAUGAAAUUAUUUCA | 1745 | UGAAAUAAUUUCAUUUAGCUU | 1657 |

TABLE 14
Exemplary human MSTN specific siRNA sequences

| Accession | | | SEQ ID | | SEQ ID |
|---|---|---|---|---|---|
| number | Position | Sense Strand | NO | Antisense Strand | NO |
| NM_005259 | 246 | UAAUGCAUGUACUUGA | 1843 | UCAAGUACAUGCAUUACACUU | 1746 |
| NM_005259 | 284 | GAAUAGAAGCCAUUAA | 1844 | UUAAUGGCUUCUAUUCUUGUU | 1747 |
| NM_005259 | 296 | UUAAGAUACAAAUCCA | 1845 | UGGAUUUGUAUCUUAAUGGUU | 1748 |
| NM_005259 | 344 | UCAGCAAAGAUGUUAA | 1846 | UUAACAUCUUUGCUGAUGUUU | 1749 |
| NM_005259 | 542 | GCUUCUUUAAAUUUAA | 1847 | UUAAAUUUAAAGAAGCAACUU | 1750 |
| NM_005259 | 585 | AAAGGCCCAACUAUGA | 1848 | UCAUAGUUGGGCCUUUACUUU | 1751 |
| NM_005259 | 622 | ACUCCUACAACAGUGA | 1849 | UCACUGUUGUAGGAGUCUCUU | 1752 |
| NM_005259 | 626 | CUACAACAGUGUUUGA | 1850 | UCAAACACUGUUGUAGGAGUU | 1753 |
| NM_005259 | 752 | UGAAGACAGUGUUGCA | 1851 | UGCAACACUGUCUUCACAUUU | 1754 |
| NM_005259 | 959 | CAGAAUCACGAUGCUA | 1852 | UAGCAUCGUGAUUCUGUUGUU | 1755 |
| NM_005259 | 1011 | AUGGGAUUGGAUUAUA | 1853 | UAUAAUCCAAUCCCAUCCAUU | 1756 |
| NM_005259 | 1015 | GAUUGGAUUAUCGCUA | 1854 | UAGCGAUAAUCCAAUCCCAUU | 1757 |
| NM_005259 | 1055 | GCUCUGGAGAGUGUGA | 1855 | UCACACUCUCCAGAGCAGUUU | 1758 |
| NM_005259 | 1061 | GAGAGUGUGAAUUUGA | 1856 | UCAAAUUCACACUCUCCAGUU | 1759 |
| NM_005259 | 1062 | AGAGUGUGAAUUUGUA | 1857 | UACAAAUUCACACUCUCCAUU | 1760 |
| NM_005259 | 1168 | CCAAUUAAUAUGCUAA | 1858 | UUAGCAUAUUAAUUGGAGAUU | 1761 |
| NM_005259 | 1169 | CAAUUAAUAUGCUAUA | 1859 | UAUAGCAUAUUAAUUGGAGUU | 1762 |
| NM_005259 | 1172 | UUAAUAUGCUAUAUUA | 1860 | UAAUAUAGCAUAUUAAUUGUU | 1763 |
| NM_005259 | 1193 | GCAAAGAACAAAUAAA | 1861 | UUUAUUUGUUCUUUGCCAUUU | 1764 |
| NM_005259 | 1194 | CAAAGAACAAAUAAUA | 1862 | UAUUAUUUGUUCUUUGCCAUU | 1765 |
| NM_005259 | 1196 | AAGAACAAAUAAUAA | 1863 | UUAUUAUUUGUUCUUUGCCU | 1766 |
| NM_005259 | 1377 | CAUAAGCUACAGUAUA | 1864 | UAUACUGUAGCUUAUGCUUUU | 1767 |
| NM_005259 | 1409 | AUAUAUGCAAUGGUUA | 1865 | UAACCAUUGCAUAUAUUCCUU | 1768 |
| NM_005259 | 1464 | UGAUUUCCAGAGUUUA | 1866 | UAAACUCUGGAAAUCAUAAUU | 1769 |
| NM_005259 | 1482 | AGCUAGAAGGAGAUCA | 1867 | UGAUCUCCUUCUAGCUCAAUU | 1770 |
| NM_005259 | 1483 | GCUAGAAGGAGAUCAA | 1868 | UUGAUCUCCUUCUAGCUCAUU | 1771 |
| NM_005259 | 1495 | UCAAAUUACAUUUAUA | 1869 | UAUAAAUGUAAUUUGAUCUUU | 1772 |
| NM_005259 | 1498 | AAUUACAUUUAUGUUA | 1870 | UAACAUAAAUGUAAUUUGAUU | 1773 |
| NM_005259 | 1500 | UUACAUUUAUGUUCCA | 1871 | UGGAACAUAAAUGUAAUUUUU | 1774 |
| NM_005259 | 1502 | ACAUUUAUGUUCCUAA | 1872 | UUAGGAACAUAAAUGUAAUUU | 1775 |
| NM_005259 | 1516 | AUAUAUUACAACAUCA | 1873 | UGAUGUUGUAAUAUAUAGGUU | 1776 |
| NM_005259 | 1538 | AAAUGAAAGCGAUUCA | 1874 | UGAAUCGCUUUCAUUUCCUUU | 1777 |
| NM_005259 | 1568 | GAAUUAAAGGAGUAUA | 1875 | UAUACUCCUUUAAUUCAUCUU | 1778 |
| NM_005259 | 1583 | GCUUUAAAGUCUAUUA | 1876 | UAAUAGACUUUAAAGCAUAUU | 1779 |
| NM_005259 | 1589 | AAGUCUAUUUCUUUAA | 1877 | UUAAAGAAAUAGACUUUAAUU | 1780 |
| NM_005259 | 1595 | AUUUCUUUAAAGUUUA | 1878 | UAAACUUUAAAGAAAUAGAUU | 1781 |
| NM_005259 | 1663 | UUAUAUACCAUCAUUA | 1879 | UAAUGAUGGUAUAUAACAAUU | 1782 |
| NM_005259 | 1663 | AUAUAUUUCUGCAUUA | 1880 | UAAUGCAGAAAUAUAUUCAUU | 1783 |
| NM_005259 | 1682 | UCAUCCUUAAACACUA | 1881 | UAGUGUUUAAGGAUGAUUCUU | 1784 |
| NM_005259 | 1691 | AACACUUGAAUUUAUA | 1882 | UAUAAAUUCAAGUGUUUAAUU | 1785 |
| NM_005259 | 1694 | ACUUGAAUUUAUAUUA | 1883 | UAAUAUAAAUUCAAGUGUUUU | 1786 |
| NM_005259 | 1698 | GAAUUUAUAUUGUAUA | 1884 | UAUACAAUAUAAAUUCAAGUU | 1787 |
| NM_005259 | 1753 | AUGGUGCAGCAUAUGA | 1885 | UCAUAUGCUGCACCAUCCCUU | 1788 |
| NM_005259 | 1761 | GCAUAUGCAAUUUCCA | 1886 | UGGAAAUUGCAUAUGCUGCUU | 1789 |
| NM_005259 | 2010 | AAACUGCAUUAUCUUA | 1887 | UAAGAUAAUGCAGUUUCUCUU | 1790 |
| NM_005259 | 2080 | AGAAGUAUGUAAUUUA | 1888 | UAAAUUACAUACUUCUCCAUU | 1791 |
| NM_005259 | 2119 | CCUUUGCAACACUGCA | 1889 | UGCAGUGUUGCAAAGGCAGUU | 1792 |
| NM_005259 | 2124 | GCAACACUGCAGUUUA | 1890 | UAAACUGCAGUGUUGCAAAUU | 1793 |
| NM_005259 | 2155 | GAAAUGAUCGACUCUA | 1891 | UAGAGUCGAUCAUUUCUAUUU | 1794 |
| NM_005259 | 2156 | AAAUGAUCGACUCUAA | 1892 | UUAGAGUCGAUCAUUUCUAUU | 1795 |
| NM_005259 | 2164 | GACUCUAUCAAUAUUA | 1893 | UAAUAUUGAUAGAGUCGAUUU | 1796 |
| NM_005259 | 2168 | CUAUCAAUAUUGUAUA | 1894 | UAUACAAUAUUGAUAGAGUUU | 1797 |
| NM_005259 | 2188 | GACUGAAACAAUGCAA | 1895 | UUGCAUUGUUUCAGUCUUUUU | 1798 |
| NM_005259 | 2193 | AAACAAUGCAUUUAUA | 1896 | UAUAAAUGCAUUGUUUCAGUU | 1799 |
| NM_005259 | 2195 | ACAAUGCAUUUAUAUA | 1897 | UAUAUAAAUGCAUUGUUUCUU | 1800 |
| NM_005259 | 2196 | CAAUGCAUUUAUAUAA | 1898 | UUAUAUAAAUGCAUUGUUUUU | 1801 |
| NM_005259 | 2209 | UAAUAUGUAUACAAUA | 1899 | UAUUGUAUACAUAUUAUAUUU | 1802 |
| NM_005259 | 2212 | UAUGUAUACAAUAUUA | 1900 | UAAUAUUGUAUACAUAUUAUU | 1803 |
| NM_005259 | 2216 | UAUACAAUAUUGUUUA | 1901 | UAAACAAUAUUGUAUACAUUU | 1804 |
| NM_005259 | 2230 | UUGUAAAUAAGUGUCA | 1902 | UGACACUUAUUUACAAAACUU | 1805 |
| NM_005259 | 2232 | GUAAAUAAGUGUCUCA | 1903 | UGAGACACUUAUUUACAAAUU | 1806 |
| NM_005259 | 2233 | UAAAUAAGUGUCUCCA | 1904 | UGGAGACACUUAUUUACAAUU | 1807 |
| NM_005259 | 2234 | AAAUAAGUGUCUCCUA | 1905 | UAGGAGACACUUAUUUACAUU | 1808 |
| NM_005259 | 2274 | ACACUAAGGACAUUUA | 1906 | UAAAUGUCCUUAGUGUAAAUU | 1809 |
| NM_005259 | 2276 | ACUAAGGACAUUUCAA | 1907 | UUGAAAUGUCCUUAGUGUAUU | 1810 |
| NM_005259 | 2283 | ACAUUUCAAAUUAAGA | 1908 | UCUUAAUUUGAAAUGUCCUUU | 1811 |
| NM_005259 | 2363 | GCAGAUUAAAUAGUGA | 1909 | UCACUAUUUAAUCUGCUAAUU | 1812 |
| NM_005259 | 2364 | CAGAUUAAAUAGUGGA | 1910 | UCCACUAUUUAAUCUGCUAUU | 1813 |
| NM_005259 | 2365 | AGAUUAAAUAGUGGUA | 1911 | UACCACUAUUUAAUCUGCUUU | 1814 |
| NM_005259 | 2397 | UAAUGAUUAGAUGGUA | 1912 | UACCAUCUAAUCAUUAACAUU | 1815 |
| NM_005259 | 2406 | GAUGGUUAUAUUACAA | 1913 | UUGUAAUAUAACCAUCUAAUU | 1816 |
| NM_005259 | 2411 | UUAUAUUACAAUCAUA | 1914 | UAUGAUUGUAAUAUAACCAUU | 1817 |
| NM_005259 | 2439 | ACAUGAUUAACAUUCA | 1915 | UGAAUGUUAAUCAUGUAAAUU | 1818 |
| NM_005259 | 2440 | CAUGAUUAACAUUCAA | 1916 | UUGAAUGUUAAUCAUGUAAUU | 1819 |
| NM_005259 | 2475 | GUAUAAAGUGAAUUUA | 1917 | UAAAUUCACUUUAUACAGCUU | 1820 |
| NM_005259 | 2491 | AAAUUUCAAUGGUUUA | 1918 | UAAACCAUUGAAAUUUCAAUU | 1821 |
| NM_005259 | 2493 | AUUUCAAUGGUUUACA | 1919 | UGUAAACCAUUGAAAUUUCUU | 1822 |
| NM_005259 | 2513 | UUGUGUUUAAAUCUCA | 1920 | UGAGAUUUAAACACAAUGAUU | 1823 |
| NM_005259 | 2519 | UUAAAUCUCAACGUUA | 1921 | UAACGUUGAGAUUUAAACAUU | 1824 |
| NM_005259 | 2521 | AAAUCUCAACGUUCCA | 1922 | UGGAACGUUGAGAUUUAAAUU | 1825 |
| NM_005259 | 2609 | AACUGAUGCUAUCUCA | 1923 | UGAGAUAGCAUCAGUUUAUUU | 1826 |
| NM_005259 | 2622 | UCAACAAUAACUGUUA | 1924 | UAACAGUUAUUGUUGAGAUUU | 1827 |
| NM_005259 | 2623 | CAACAAUAACUGUUAA | 1925 | UUAACAGUUAUUGUUGAGAUU | 1828 |
| NM_005259 | 2649 | UAAUUUGAUAAUGAAA | 1926 | UUUCAUUAUCAAAUUAUAAUU | 1829 |
| NM_005259 | 2657 | UAAUGAAUAUAUUUCA | 1927 | UGAAAUAUAUUCAUUAUCAUU | 1830 |
| NM_005259 | 2658 | AAUGAAUAUAUUUCUA | 1928 | UAGAAAUAUAUUCAUUAUCUU | 1831 |
| NM_005259 | 2661 | GAAUAUAUUUCUGCAA | 1929 | UUGCAGAAAUAUAUUCAUUUU | 1832 |
| NM_005259 | 2671 | CUGCAUUUAUUUACUA | 1930 | UAGUAAAUAAAUGCAGAAAUU | 1833 |
| NM_005259 | 2675 | AUUUAUUUACUUCUGA | 1931 | UCAGAAGUAAAUAAAUGCAUU | 1834 |
| NM_005259 | 2711 | UAAUCAAAUUUAUUGA | 1932 | UCAAUAAAUUUGAUUAACAUU | 1835 |
| NM_005259 | 2733 | ACUAAAUGAAAUUAUA | 1933 | UAUAAUUUCAUUUAGUCAUUU | 1836 |
| NM_005259 | 2734 | CUAAAUGAAAUUAUUA | 1934 | UAAUAAUUUCAUUUAGUCAUU | 1837 |
| NM_005259 | 2750 | CUUACAUCUAAUUUGA | 1935 | UCAAAUUAGAUGUAAGAAAUU | 1838 |
| NM_005259 | 2753 | ACAUCUAAUUUGUAGA | 1936 | UCUACAAAUUAGAUGUAAGUU | 1839 |
| NM_005259 | 2765 | UAGAAACAGUAUAAGA | 1937 | UCUUAUACUGUUUCUACAAUU | 1840 |
| NM_005259 | 2767 | GAAACAGUAUAAGUUA | 1938 | UAACUUAUACUGUUUCUACUU | 1841 |
| NM_005259 | 2770 | ACAGUAUAAGUUAUAA | 1939 | UUAUAACUUAUACUGUUUCUU | 1842 |

In some embodiments, a mouse Mstn specific siRNA comprises an antisense strand that comprises at least 15 consecutive nucleosides of (e.g., at least 15, at least 16, at least 17, at least 18, at least 19, or at least 20) the sequence of any one of SEQ ID NOs: 1570-1657. In some embodiments, a mouse Mstn specific siRNA further comprises a sense strand that comprises at least 15 consecutive nucleosides (e.g., at least 15, at least 16, at least 17, at least 18, at least 19, or at least 20) complementary to the sequence of any one of SEQ ID NOs: 1570-1657. In some embodiments, a mouse Mstn siRNA further comprises a sense strand that comprises at least 15 consecutive nucleosides of (e.g., at least 15, at least 16, at least 17, at least 18, at least 19, or at least 20) the sequence of any one of SEQ ID NOs: 1658-1745.

In some embodiments, a mouse Mstn specific siRNA comprises an antisense strand that is at least 80% (e.g., at least 85%, at least 90%, at least 95%, at least 96%, at least 97%, at least 98%, at least 99%, or 100%) to the sequence of any one of SEQ ID NOs: 1570-1657. In some embodiments, a mouse Mstn specific siRNA further comprises a sense strand that comprises a complementary to a sequence at least 80% (e.g., at least 85%, at least 90%, at least 95%, at least 96%, at least 97%, at least 98%, at least 99%, or 100%) identical to the sequence of any one of SEQ ID NOs: 1570-1657. In some embodiments, a mouse Mstn specific siRNA further comprises a sense strand that is at least 80% (e.g., at least 85%, at least 90%, at least 95%, at least 96%, at least 97%, at least 98%, at least 99%, or 100%) identical to the sequence of any one of SEQ ID NOs: 1658-1745.

In some embodiments, a human MSTN specific siRNA comprises an antisense strand that comprises at least 15 consecutive nucleosides of (e.g., at least 15, at least 16, at least 17, at least 18, at least 19, or at least 20) the sequence of any one of SEQ ID NOs: 1746-1842. In some embodiments, a human MSTN specific siRNA further comprises a sense strand that comprises at least 15 consecutive nucleosides (e.g., at least 15, at least 16, at least 17, at least 18, at least 19, or at least 20) complementary to the sequence of any one of SEQ ID NOs: 1746-1842. In some embodiments, a human MSTN siRNA further comprises a sense strand that comprises at least 15 consecutive nucleosides of (e.g., at least 15, at least 16, at least 17, at least 18, at least 19, or at least 20) the sequence of any one of SEQ ID NOs: 1843-1939.

In some embodiments, a human MSTN specific siRNA comprises an antisense strand that is at least 80% (e.g., at least 85%, at least 90%, at least 95%, at least 96%, at least 97%, at least 98%, at least 99%, or 100%) to the sequence of any one of SEQ ID NOs: 1746-1842. In some embodiments, a human MSTN specific siRNA further comprises a sense strand that comprises a complementary to a sequence at least 80% (e.g., at least 85%, at least 90%, at least 95%, at least 96%, at least 97%, at least 98%, at least 99%, or 100%) identical to the sequence of any one of SEQ ID NOs: 1746-1842. In some embodiments, a human MSTN specific siRNA further comprises a sense strand that is at least 80% (e.g., at least 85%, at least 90%, at least 95%, at least 96%, at least 97%, at least 98%, at least 99%, or 100%) identical to the sequence of any one of SEQ ID NOs: 1843-1939.

In some embodiments, an MSTN specific siRNA described herein comprises at least one nucleoside modified at the 2′ position of the sugar. In some embodiments, an MSTN specific siRNA comprises at least one 2′-modified nucleoside. In some embodiments, all of the nucleosides in the oligonucleotide are 2′-modified nucleosides.

In some embodiments, an MSTN specific siRNA described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside. In some embodiments, an MSTN specific siRNA comprises one or more 2′-O-methoxyethyl (2′-MOE) modified nucleoside and/or one or more 2′-fluoro (2′-F) modified nucleoside.

In some embodiments, an MSTN specific siRNA comprises one or more modified one or more modified internucleotide linkages. In some embodiments, the internucleotide linkage is a phosphorothioate linkage. In some embodiments, an MSTN specific siRNA further comprises a cholesterol molecule. In some embodiments, an MSTN specific siRNA is conjugated to the cholesterol by a TEG linker. In some embodiments, an MSTN specific siRNA is conjugated to a docosanoic acid (DCA). In some embodiments, the DCA is conjugated to the isolated nucleic acid via a cleavable linker. Exemplary an MSTN specific siRNA with chemical modifications are set forth in Tables 4 and 5.

TABLE 4
Modified mouse Mstn specific siRNA

| | SEQ |
|---|---|
| | ID |
| | NO |
| Sense Strand | |
| (mA)#(mG)#(mA)(fA)(mA)(fU)(mA)(fA)(mG)(fA)(mA)(mC)(mA)(fA)#(mG)#(mA)-TegChol | 285 |
| (mU)#(mA)#(mA)(fU)(mG)(fC)(mA)(fU)(mG)(fU)(mG)(mC)(mG)(fU)#(mG)#(mA)-TegChol | 286 |
| (mC)#(mC)#(mA)(fG)(mA)(fA)(mU)(fA)(mG)(fA)(mA)(mG)(mC)(fC)#(mA)#(mA)-TegChol | 287 |
| (mG)#(mC)#(mA)(fA)(mG)(fC)(mC)(fC)(mA)(fA)(mA)(mU)(mG)(fU)#(mU)#(mA)-TegChol | 288 |
| (mC)#(mA)#(mA)(fG)(mC)(fC)(mC)(fA)(mA)(fA)(mU)(mG)(mU)(fU)#(mG)#(mA)-TegChol | 289 |
| (mA)#(mC)#(mU)(fC)(mC)(fU)(mA)(fC)(mA)(fA)(mC)(mA)(mG)(fU)#(mG)#(mA)-TegChol | 290 |
| (mC)#(mC)#(mU)(fG)(mA)(fA)(mU)(fC)(mC)(fA)(mA)(mC)(mU)(fU)#(mA)#(mA)-TegChol | 291 |
| (mA)#(mU)#(mG)(fG)(mG)(fA)(mC)(fU)(mG)(fG)(mA)(mU)(mU)(fA)#(mU)#(mA)-TegChol | 292 |
| (mG)#(mC)#(mU)(fC)(mA)(fG)(mG)(fA)(mG)(fA)(mG)(mU)(mG)(fU)#(mG)#(mA)-TegChol | 293 |
| (mG)#(mA)#(mG)(fA)(mG)(fU)(mG)(fU)(mG)(fA)(mA)(mU)(mU)(fU)#(mG)#(mA)-TegChol | 294 |
| (mA)#(mG)#(mA)(fG)(mU)(fG)(mU)(fG)(mA)(fA)(mU)(mU)(mU)(fG)#(mU)#(mA)-TegChol | 295 |
| (mU)#(mU)#(mA)(fA)(mU)(fA)(mU)(fG)(mC)(fU)(mA)(mU)(mA)(fU)#(mU)#(mA)-TegChol | 296 |
| (mG)#(mC)#(mA)(fA)(mA)(fG)(mA)(fA)(mC)(fA)(mA)(mA)(mU)(fA)#(mA)#(mA)-TegChol | 297 |
| (mC)#(mA)#(mA)(fA)(mG)(fA)(mA)(fC)(mA)(fA)(mA)(mU)(mA)(fA)#(mU)#(mA)-TegChol | 298 |
| (fA)#(mA)#(fG)(mA)(fA)(mC)(fA)(mA)(fA)(mU)(fA)(mA)(fU)#(mA)#(fA)-TegChol | 299 |
| (mU)#(mU)#(mA)(fG)(mG)(fU)(mU)(fA)(mG)(fA)(mA)(mA)(mU)(fU)#(mU)#(mA)-TegChol | 300 |
| (mG)#(mC)#(mA)(fC)(mC)(fA)(mC)(fA)(mG)(fG)(mC)(mU)(mG)(fU)#(mA)#(mA)-TegChol | 301 |
| (mC)#(mA)#(mA)(fG)(mC)(fU)(mA)(fC)(mA)(fG)(mU)(mG)(mU)(fA)#(mU)#(mA)-TegChol | 302 |
| (mG)#(mC)#(mA)(fU)(mU)(fC)(mA)(fA)(mC)(fC)(mA)(mC)(mC)(fA)#(mA)#(mA)-TegChol | 303 |
| (mA)#(mU)#(mG)(fU)(mU)(fG)(mU)(fA)(mU)(fG)(mA)(mU)(mU)(fU)#(mC)#(mA)-TegChol | 304 |
| (mG)#(mU)#(mA)(fU)(mG)(fA)(mU)(fU)(mU)(fC)(mC)(mA)(mG)(fA)#(mG)#(mA)-TegChol | 305 |
| (mA)#(mG)#(mA)(fU)(mC)(fA)(mA)(fA)(mU)(fU)(mA)(mC)(mA)(fU)#(mU)#(mA)-TegChol | 306 |
| (mC)#(mA)#(mA)(fA)(mU)(fU)(mA)(fC)(mA)(fU)(mU)(mU)(mA)(fU)#(mG)#(mA)-TegChol | 307 |
| (mU)#(mU)#(mA)(fU)(mU)(fU)(mC)(fC)(mU)(fA)(mA)(mC)(mA)(fG)#(mU)#(mA)-TegChol | 308 |
| (mA)#(mU)#(mU)(fU)(mC)(fC)(mU)(fA)(mA)(fC)(mA)(mG)(mU)(fU)#(mU)#(mA)-TegChol | 309 |
| (mU)#(mU)#(mU)(fC)(mC)(fU)(mA)(fA)(mC)(fA)(mG)(mU)(mU)(fU)#(mC)#(mA)-TegChol | 310 |
| (mG)#(mA)#(mA)(fG)(mA)(fA)(mU)(fC)(mU)(fA)(mU)(mA)(mU)(fG)#(mU)#(mA)-TegChol | 311 |
| (mA)#(mA)#(mG)(fA)(mA)(fU)(mC)(fU)(mA)(fU)(mA)(mU)(mG)(fU)#(mA)#(mA)-TegChol | 312 |
| (mG)#(mA)#(mA)(fU)(mC)(fU)(mA)(fU)(mA)(fU)(mG)(mU)(mA)(fG)#(mC)#(mA)-TegChol | 313 |
| (mA)#(mA)#(mA)(fG)(mU)(fG)(mU)(fA)(mG)(fG)(mA)(mU)(mU)(fG)#(mU)#(mA)-TegChol | 314 |
| (mA)#(mA)#(mG)(fU)(mG)(fU)(mA)(fG)(mG)(fA)(mU)(mU)(mG)(fU)#(mU)#(mA)-TegChol | 315 |
| (mU)#(mA)#(mC)(fA)(mC)(fU)(mU)(fA)(mU)(fA)(mU)(mU)(mU)(fG)#(mU)#(mA)-TegChol | 316 |
| (mU)#(mU)#(mA)(fU)(mA)(fU)(mU)(fU)(mG)(fU)(mA)(mU)(mU)(fG)#(mU)#(mA)-TegChol | 317 |
| (mG)#(mU)#(mA)(fU)(mU)(fG)(mU)(fA)(mU)(fA)(mC)(mU)(mU)(fG)#(mG)#(mA)-TegChol | 318 |
| (mU)#(mA)#(mU)(fU)(mG)(fU)(mA)(fU)(mA)(fC)(mU)(mU)(mG)(fG)#(mU)#(mA)-TegChol | 319 |
| (mA)#(mC)#(mA)(fA)(mA)(fG)(mU)(fA)(mG)(fG)(mA)(mA)(mU)(fG)#(mG)#(mA)-TegChol | 320 |
| (mG)#(mG)#(mA)(fA)(mA)(fG)(mA)(fA)(mG)(fA)(mA)(mU)(mC)(fU)#(mU)#(mA)-TegChol | 321 |
| (mC)#(mA)#(mG)(fC)(mC)(fA)(mA)(fA)(mC)(fA)(mG)(mU)(mA)(fU)#(mU)#(mA)-TegChol | 322 |
| (mC)#(mC)#(mU)(fU)(mU)(fA)(mU)(fC)(mA)(fC)(mA)(mU)(mG)(fG)#(mC)#(mA)-TegChol | 323 |
| (mA)#(mA)#(mA)(fU)(mG)(fA)(mC)(fU)(mG)(fG)(mU)(mU)(mC)(fU)#(mA)#(mA)-TegChol | 324 |
| (mC)#(mU)#(mC)(fU)(mG)(fA)(mA)(fA)(mC)(fA)(mA)(mU)(mU)(fA)#(mC)#(mA)-TegChol | 325 |
| (mA)#(mA)#(mA)(fC)(mA)(fA)(mU)(fU)(mA)(fC)(mA)(mU)(mU)(fU)#(mA)#(mA)-TegChol | 326 |
| (mA)#(mA)#(mC)(fA)(mA)(fU)(mU)(fA)(mC)(fA)(mU)(mU)(mU)(fA)#(mU)#(mA)-TegChol | 327 |
| (mA)#(mC)#(mA)(fA)(mU)(fU)(mA)(fC)(mA)(fU)(mU)(mU)(mA)(fU)#(mA)#(mA)-TegChol | 328 |
| (mU)#(mU)#(mA)(fU)(mA)(fU)(mA)(fA)(mU)(fA)(mU)(mG)(mU)(fA)#(mU)#(mA)-TegChol | 329 |
| (mU)#(mA)#(mU)(fG)(mU)(fA)(mU)(fA)(mC)(fA)(mA)(mU)(mA)(fU)#(mU)#(mA)-TegChol | 330 |
| (mA)#(mU)#(mG)(fU)(mA)(fU)(mA)(fC)(mA)(fA)(mU)(mA)(mU)(fU)#(mG)#(mA)-TegChol | 331 |
| (mU)#(mA)#(mU)(fA)(mC)(fA)(mA)(fU)(mA)(fU)(mU)(mG)(mU)(fU)#(mU)#(mA)-TegChol | 332 |
| (mU)#(mU)#(mG)(fU)(mA)(fA)(mA)(fU)(mA)(fA)(mG)(mU)(mG)(fU)#(mC)#(mA)-TegChol | 333 |
| (mG)#(mU)#(mA)(fA)(mA)(fU)(mA)(fA)(mG)(fU)(mG)(mU)(mC)(fU)#(mC)#(mA)-TegChol | 334 |
| (mU)#(mA)#(mA)(fA)(mU)(fA)(mA)(fG)(mU)(fG)(mU)(mC)(mU)(fC)#(mC)#(mA)-TegChol | 335 |
| (mA)#(mA)#(mA)(fU)(mA)(fA)(mG)(fU)(mG)(fU)(mC)(mU)(mC)(fC)#(mU)#(mA)-TegChol | 336 |
| (mA)#(mU)#(mA)(fA)(mG)(fU)(mG)(fU)(mC)(fU)(mC)(mC)(mU)(fU)#(mU)#(mA)-TegChol | 337 |
| (mA)#(mC)#(mA)(fC)(mU)(fA)(mA)(fU)(mG)(fA)(mA)(mA)(mU)(fU)#(mU)#(mA)-TegChol | 338 |
| (mC)#(mA)#(mC)(fU)(mA)(fA)(mU)(fG)(mA)(fA)(mA)(mU)(mU)(fU)#(mC)#(mA)-TegChol | 339 |
| (mA)#(mC)#(mU)(fA)(mA)(fU)(mG)(fA)(mA)(fA)(mU)(mU)(mU)(fC)#(mA)#(mA)-TegChol | 340 |
| (mA)#(mA)#(mA)(fU)(mU)(fU)(mC)(fA)(mA)(fA)(mU)(mC)(mA)(fU)#(mU)#(mA)-TegChol | 341 |
| (mU)#(mC)#(mA)(fU)(mU)(fA)(mA)(fA)(mG)(fU)(mA)(mC)(mA)(fA)#(mA)#(mA)-TegChol | 342 |
| (mG)#(mC)#(mA)(fG)(mA)(fU)(mU)(fC)(mA)(fA)(mU)(mA)(mG)(fU)#(mG)#(mA)-TegChol | 343 |
| (mA)#(mG)#(mA)(fU)(mU)(fC)(mA)(fA)(mU)(fA)(mG)(mU)(mG)(fG)#(mU)#(mA)-TegChol | 344 |
| (mU)#(mU)#(mA)(fA)(mG)(fA)(mU)(fU)(mA)(fG)(mA)(mA)(mG)(fG)#(mU)#(mA)-TegChol | 345 |
| (mA)#(mG)#(mA)(fU)(mU)(fA)(mG)(fA)(mA)(fG)(mG)(mU)(mU)(fA)#(mU)#(mA)-TegChol | 346 |
| (mA)#(mU)#(mU)(fA)(mG)(fA)(mA)(fG)(mG)(fU)(mU)(mA)(mU)(fA)#(mU)#(mA)-TegChol | 347 |
| (mU)#(mU)#(mA)(fU)(mA)(fU)(mU)(fA)(mC)(fA)(mA)(mU)(mC)(fA)#(mA)#(mA)-TegChol | 348 |
| (mA)#(mU)#(mA)(fU)(mU)(fA)(mC)(fA)(mA)(fU)(mC)(mA)(mA)(fU)#(mU)#(mA)-TegChol | 349 |
| (mA)#(mU)#(mU)(fA)(mC)(fA)(mA)(fU)(mC)(fA)(mA)(mU)(mU)(fU)#(mA)#(mA)-TegChol | 350 |
| (mA)#(mC)#(mA)(fA)(mU)(fC)(mA)(fA)(mU)(fU)(mU)(mA)(mU)(fG)#(mU)#(mA)-TegChol | 351 |
| (mC)#(mA)#(mA)(fU)(mC)(fA)(mA)(fU)(mU)(fU)(mA)(mU)(mG)(fU)#(mA)#(mA)-TegChol | 352 |
| (mC)#(mA)#(mU)(fU)(mA)(fU)(mC)(fA)(mA)(fC)(mA)(mU)(mU)(fC)#(mA)#(mA)-TegChol | 353 |
| (mA)#(mA)#(mA)(fC)(mA)(fU)(mA)(fC)(mC)(fA)(mA)(mC)(mA)(fU)#(mG)#(mA)-TegChol | 354 |
| (mA)#(mC)#(mA)(fU)(mA)(fC)(mC)(fA)(mA)(fC)(mA)(mU)(mG)(fU)#(mA)#(mA)-TegChol | 355 |
| (mA)#(mC)#(mA)(fU)(mG)(fU)(mA)(fU)(mC)(fU)(mA)(mA)(mA)(fG)#(mA)#(mA)-TegChol | 356 |
| (mC)#(mA)#(mA)(fA)(mU)(fA)(mU)(fG)(mG)(fU)(mA)(mU)(mC)(fU)#(mC)#(mA)-TegChol | 357 |
| (mG)#(mU)#(mA)(fU)(mC)(fU)(mC)(fA)(mA)(fU)(mA)(mA)(mC)(fA)#(mG)#(mA)-TegChol | 358 |
| (mU)#(mC)#(mA)(fA)(mU)(fA)(mA)(fC)(mA)(fG)(mC)(mU)(mA)(fC)#(mU)#(mA)-TegChol | 359 |
| (mC)#(mA)#(mA)(fU)(mA)(fA)(mC)(fA)(mG)(fC)(mU)(mA)(mC)(fU)#(mU)#(mA)-TegChol | 360 |
| (mA)#(mA)#(mU)(fA)(mA)(fC)(mA)(fG)(mC)(fU)(mA)(mC)(mU)(fU)#(mU)#(mA)-TegChol | 361 |
| (mU)#(mA)#(mA)(fU)(mU)(fU)(mG)(fA)(mC)(fA)(mA)(mU)(mG)(fA)#(mA)#(mA)-TegChol | 362 |
| (mA)#(mC)#(mA)(fA)(mU)(fG)(mA)(fA)(mU)(fA)(mC)(mA)(mU)(fU)#(mU)#(mA)-TegChol | 363 |
| (mC)#(mA)#(mA)(fU)(mG)(fA)(mA)(fU)(mA)(fC)(mA)(mU)(mU)(fU)#(mC)#(mA)-TegChol | 364 |
| (mA)#(mA)#(mU)(fG)(mA)(fA)(mU)(fA)(mC)(fA)(mU)(mU)(mU)(fC)#(mU)#(mA)-TegChol | 365 |
| (mA)#(mU)#(mA)(fA)(mA)(fU)(mU)(fG)(mG)(fA)(mA)(mC)(mU)(fU)#(mU)#(mA)-TegChol | 366 |
| (mU)#(mA)#(mA)(fA)(mU)(fU)(mG)(fG)(mA)(fA)(mC)(mU)(mU)(fU)#(mG)#(mA)-TegChol | 367 |
| (mA)#(mA)#(mA)(fU)(mU)(fG)(mG)(fA)(mA)(fC)(mU)(mU)(mU)(fG)#(mU)#(mA)-TegChol | 368 |
| (mA)#(mC)#(mU)(fU)(mU)(fG)(mU)(fU)(mU)(fA)(mU)(mC)(mA)(fA)#(mA)#(mA)-TegChol | 369 |
| (mU)#(mC)#(mA)(fU)(mA)(fG)(mC)(fU)(mA)(fA)(mA)(mU)(mG)(fA)#(mA)#(mA)-TegChol | 370 |
| (mC)#(mU)#(mA)(fA)(mA)(fU)(mG)(fA)(mA)(fA)(mU)(mU)(mA)(fU)#(mU)#(mA)-TegChol | 371 |
| (mA)#(mA)#(mA)(fU)(mG)(fA)(mA)(fA)(mU)(fU)(mA)(mU)(mU)(fU)#(mC)#(mA)-TegChol | 372 |
| Antisense Strand | |
| P(mU)#(fC)#(mU)(fU)(fG)(fU)(mU)(fC)(mU)(fU)(mA)(fU)(mU)(fU)#(mC)#(fU)#(mU)#(mC)#(mU)#(mU)#(fU) | 373 |
| P(mU)#(fC)#(mA)(fC)(fG)(fC)(mA)(fC)(mA)(fU)(mG)(fC)(mA)(fU)#(mU)#(fA)#(mC)#(mA)#(mC)#(mU)#(fU) | 374 |
| P(mU)#(fU)#(mG)(fG)(fC)(fU)(mU)(fC)(mU)(fA)(mU)(fU)(mC)(fU)#(mG)#(fG)#(mA)#(mG)#(mU)#(mU)#(fU) | 375 |
| P(mU)#(fA)#(mA)(fC)(fA)(fU)(mU)(fU)(mG)(fG)(mG)(fC)(mU)(fU)#(mG)#(fC)#(mC)#(mA)#(mU)#(mU)#(fU) | 376 |
| P(mU)#(fC)#(mA)(fA)(fC)(fA)(mU)(fU)(mU)(fG)(mG)(fG)(mC)(fU)#(mU)#(fG)#(mC)#(mC)#(mA)#(mU)#(fU) | 377 |
| P(mU)#(fC)#(mA)(fC)(fU)(fG)(mU)(fU)(mG)(fU)(mA)(fG)(mG)(fA)#(mG)#(fU)#(mC)#(mU)#(mU)#(mU)#(fU) | 378 |
| P(mU)#(fU)#(mA)(fA)(fG)(fU)(mU)(fG)(mG)(fA)(mU)(fU)(mC)(fA)#(mG)#(fG)#(mC)#(mU)#(mG)#(mU)#(fU) | 379 |
| P(mU)#(fA)#(mU)(fA)(fA)(fU)(mC)(fC)(mA)(fG)(mU)(fC)(mC)(fC)#(mA)#(fU)#(mC)#(mC)#(mA)#(mU)#(fU) | 400 |
| P(mU)#(fC)#(mA)(fC)(fA)(fC)(mU)(fC)(mU)(fC)(mC)(fU)(mG)(fA)#(mG)#(fC)#(mA)#(mG)#(mU)#(mU)#(fU) | 401 |
| P(mU)#(fC)#(mA)(fA)(fA)(fU)(mU)(fC)(mA)(fC)(mA)(fC)(mU)(fC)#(mU)#(fC)#(mC)#(mU)#(mG)#(mU)#(fU) | 402 |
| P(mU)#(fA)#(mC)(fA)(fA)(fA)(mU)(fU)(mC)(fA)(mC)(fA)(mC)(fU)#(mC)#(fU)#(mC)#(mC)#(mU)#(mU)#(fU) | 403 |
| P(mU)#(fA)#(mA)(fU)(fA)(fU)(mA)(fG)(mC)(fA)(mU)(fA)(mU)(fU)#(mA)#(fA)#(mU)#(mG)#(mG)#(mU)#(fU) | 404 |
| P(mU)#(fU)#(mU)(fA)(fU)(fU)(mU)(fG)(mU)(fU)(mC)(fU)(mU)(fU)#(mG)#(fC)#(mC)#(mA)#(mU)#(mU)#(fU) | 405 |
| P(mU)#(fA)#(mU)(fU)(fA)(fU)(mU)(fU)(mG)(fU)(mU)(fC)(mU)(fU)#(mU)#(fG)#(mC)#(mC)#(mA)#(mU)#(fU) | 406 |
| P(mU)#(fU)#(mA)(fU)(mU)(fA)(mU)(fU)(mU)(fG)(mU)(fU)(mC)#(fU)#(mU)#(fU)#(mG)#(fC)#(mC)#(fU) | 407 |
| P(mU)#(fA)#(mA)(fA)(fU)(fU)(mU)(fC)(mU)(fA)(mA)(fC)(mC)(fU)#(mA)#(fA)#(mU)#(mG)#(mC)#(mU)#(fU) | 408 |
| P(mU)#(fU)#(mA)(fC)(fA)(fG)(mC)(fC)(mU)(fG)(mU)(fG)(mG)(fU)#(mG)#(fC)#(mU)#(mU)#(mG)#(mU)#(fU) | 409 |
| P(mU)#(fA)#(mU)(fA)(fC)(fA)(mC)(fU)(mG)(fU)(mA)(fG)(mC)(fU)#(mU)#(fG)#(mU)#(mG)#(mC)#(mU)#(fU) | 410 |
| P(mU)#(fU)#(mU)(fG)(fG)(fU)(mG)(fG)(mU)(fU)(mG)(fA)(mA)(fU)#(mG)#(fC)#(mC)#(mA)#(mA)#(mU)#(fU) | 411 |
| P(mU)#(fG)#(mA)(fA)(fA)(fU)(mC)(fA)(mU)(fA)(mC)(fA)(mA)(fC)#(mA)#(fU)#(mC)#(mC)#(mU)#(mU)#(fU) | 412 |
| P(mU)#(fC)#(mU)(fC)(fU)(fG)(mG)(fA)(mA)(fA)(mU)(fC)(mA)(fU)#(mA)#(fC)#(mA)#(mA)#(mC)#(mU)#(fU) | 413 |
| P(mU)#(fA)#(mA)(fU)(fG)(fU)(mA)(fA)(mU)(fU)(mU)(fG)(mA)(fU)#(mC)#(fU)#(mC)#(mC)#(mA)#(mU)#(fU) | 414 |
| P(mU)#(fC)#(mA)(fU)(fA)(fA)(mA)(fU)(mG)(fU)(mA)(fA)(mU)(fU)#(mU)#(fG)#(mA)#(mU)#(mC)#(mU)#(fU) | 415 |
| P(mU)#(fA)#(mC)(fU)(fG)(fU)(mU)(fA)(mG)(fG)(mA)(fA)(mA)(fU)#(mA)#(fA)#(mG)#(mA)#(mC)#(mU)#(fU) | 416 |
| P(mU)#(fA)#(mA)(fA)(fC)(fU)(mG)(fU)(mU)(fA)(mG)(fG)(mA)(fA)#(mA)#(fU)#(mA)#(mA)#(mG)#(mU)#(fU) | 417 |
| P(mU)#(fG)#(mA)(fA)(fA)(fC)(mU)(fG)(mU)(fU)(mA)(fG)(mG)(fA)#(mA)#(fA)#(mU)#(mA)#(mA)#(mU)#(fU) | 418 |
| P(mU)#(fA)#(mC)(fA)(fU)(fA)(mU)(fA)(mG)(fA)(mU)(fU)(mC)(fU)#(mU)#(fC)#(mC)#(mG)#(mU)#(mU)#(fU) | 419 |
| P(mU)#(fU)#(mA)(fC)(fA)(fU)(mA)(fU)(mA)(fG)(mA)(fU)(mU)(fC)#(mU)#(fU)#(mC)#(mC)#(mG)#(mU)#(fU) | 420 |
| P(mU)#(fG)#(mC)(fU)(fA)(fC)(mA)(fU)(mA)(fU)(mA)(fG)(mA)(fU)#(mU)#(fC)#(mU)#(mU)#(mC)#(mU)#(fU) | 421 |
| P(mU)#(fA)#(mC)(fA)(fA)(fU)(mC)(fC)(mU)(fA)(mC)(fA)(mC)(fU)#(mU)#(fU)#(mA)#(mC)#(mA)#(mU)#(fU) | 422 |
| P(mU)#(fA)#(mA)(fC)(fA)(fA)(mU)(fC)(mC)(fU)(mA)(fC)(mA)(fC)#(mU)#(fU)#(mU)#(mA)#(mC)#(mU)#(fU) | 423 |
| P(mU)#(fA)#(mC)(fA)(fA)(fA)(mU)(fA)(mU)(fA)(mA)(fG)(mU)(fG)#(mU)#(fA)#(mC)#(mA)#(mU)#(mU)#(fU) | 424 |
| P(mU)#(fA)#(mC)(fA)(fA)(fU)(mA)(fC)(mA)(fA)(mA)(fU)(mA)(fU)#(mA)#(fA)#(mG)#(mU)#(mG)#(mU)#(fU) | 425 |
| P(mU)#(fC)#(mC)(fA)(fA)(fG)(mU)(fA)(mU)(fA)(mC)(fA)(mA)(fU)#(mA)#(fC)#(mA)#(mA)#(mA)#(mU)#(fU) | 426 |
| P(mU)#(fA)#(mC)(fC)(fA)(fA)(mG)(fU)(mA)(fU)(mA)(fC)(mA)(fA)#(mU)#(fA)#(mC)#(mA)#(mA)#(mU)#(fU) | 427 |
| P(mU)#(fC)#(mC)(fA)(fU)(fU)(mC)(fC)(mU)(fA)(mC)(fU)(mU)(fU)#(mG)#(fU)#(mG)#(mG)#(mA)#(mU)#(fU) | 428 |
| P(mU)#(fA)#(mA)(fG)(fA)(fU)(mU)(fC)(mU)(fU)(mC)(fU)(mU)(fU)#(mC)#(fC)#(mU)#(mU)#(mA)#(mU)#(fU) | 429 |
| P(mU)#(fA)#(mA)(fU)(fA)(fC)(mU)(fG)(mU)(fU)(mU)(fG)(mG)(fC)#(mU)#(fG)#(mC)#(mC)#(mU)#(mU)#(fU) | 430 |
| P(mU)#(fG)#(mC)(fC)(fA)(fU)(mG)(fU)(mG)(fA)(mU)(fA)(mA)(fA)#(mG)#(fG)#(mC)#(mA)#(mG)#(mU)#(fU) | 431 |
| P(mU)#(fU)#(mA)(fG)(fA)(fA)(mC)(fC)(mA)(fG)(mU)(fC)(mA)(fU)#(mU)#(fU)#(mC)#(mC)#(mA)#(mU)#(fU) | 432 |
| P(mU)#(fG)#(mU)(fA)(fA)(fU)(mU)(fG)(mU)(fU)(mU)(fC)(mA)(fG)#(mA)#(fG)#(mU)#(mC)#(mU)#(mU)#(fU) | 433 |
| P(mU)#(fU)#(mA)(fA)(fA)(fU)(mG)(fU)(mA)(fA)(mU)(fU)(mG)(fU)#(mU)#(fU)#(mC)#(mA)#(mG)#(mU)#(fU) | 434 |
| P(mU)#(fA)#(mU)(fA)(fA)(fA)(mU)(fG)(mU)(fA)(mA)(fU)(mU)(fG)#(mU)#(fU)#(mU)#(mC)#(mA)#(mU)#(fU) | 435 |
| P(mU)#(fU)#(mA)(fU)(fA)(fA)(mA)(fU)(mG)(fU)(mA)(fA)(mU)(fU)#(mG)#(fU)#(mU)#(mU)#(mC)#(mU)#(fU) | 436 |
| P(mU)#(fA)#(mU)(fA)(fC)(fA)(mU)(fA)(mU)(fU)(mA)(fU)(mA)(fU)#(mA)#(fA)#(mA)#(mU)#(mG)#(mU)#(fU) | 437 |
| P(mU)#(fA)#(mA)(fU)(fA)(fU)(mU)(fG)(mU)(fA)(mU)(fA)(mC)(fA)#(mU)#(fA)#(mU)#(mU)#(mA)#(mU)#(fU) | 438 |
| P(mU)#(fC)#(mA)(fA)(fU)(fA)(mU)(fU)(mG)(fU)(mA)(fU)(mA)(fC)#(mA)#(fU)#(mA)#(mU)#(mU)#(mU)#(fU) | 439 |
| P(mU)#(fA)#(mA)(fA)(fC)(fA)(mA)(fU)(mA)(fU)(mU)(fG)(mU)(fA)#(mU)#(fA)#(mC)#(mA)#(mU)#(mU)#(fU) | 440 |
| P(mU)#(fG)#(mA)(fC)(fA)(fC)(mU)(fU)(mA)(fU)(mU)(fU)(mA)(fC)#(mA)#(fA)#(mA)#(mA)#(mC)#(mU)#(fU) | 441 |
| P(mU)#(fG)#(mA)(fG)(fA)(fC)(mA)(fC)(mU)(fU)(mA)(fU)(mU)(fU)#(mA)#(fC)#(mA)#(mA)#(mA)#(mU)#(fU) | 442 |
| P(mU)#(fG)#(mG)(fA)(fG)(fA)(mC)(fA)(mC)(fU)(mU)(fA)(mU)(fU)#(mU)#(fA)#(mC)#(mA)#(mA)#(mU)#(fU) | 443 |
| P(mU)#(fA)#(mG)(fG)(fA)(fG)(mA)(fC)(mA)(fC)(mU)(fU)(mA)(fU)#(mU)#(fU)#(mA)#(mC)#(mA)#(mU)#(fU) | 444 |
| P(mU)#(fA)#(mA)(fA)(fG)(fG)(mA)(fG)(mA)(fC)(mA)(fC)(mU)(fU)#(mA)#(fU)#(mU)#(mU)#(mA)#(mU)#(fU) | 445 |
| P(mU)#(fA)#(mA)(fA)(fU)(fU)(mU)(fC)(mA)(fU)(mU)(fA)(mG)(fU)#(mG)#(fU)#(mA)#(mA)#(mA)#(mU)#(fU) | 446 |
| P(mU)#(fG)#(mA)(fA)(fA)(fU)(mU)(fU)(mC)(fA)(mU)(fU)(mA)(fG)#(mU)#(fG)#(mU)#(mA)#(mA)#(mU)#(fU) | 447 |
| P(mU)#(fU)#(mG)(fA)(fA)(fA)(mU)(fU)(mU)(fC)(mA)(fU)(mU)(fA)#(mG)#(fU)#(mG)#(mU)#(mA)#(mU)#(fU) | 448 |
| P(mU)#(fA)#(mA)(fU)(fG)(fA)(mU)(fU)(mU)(fG)(mA)(fA)(mA)(fU)#(mU)#(fU)#(mC)#(mA)#(mU)#(mU)#(fU) | 449 |
| P(mU)#(fU)#(mU)(fU)(fG)(fU)(mA)(fC)(mU)(fU)(mU)(fA)(mA)(fU)#(mG)#(fA)#(mU)#(mU)#(mU)#(mU)#(fU) | 450 |
| P(mU)#(fC)#(mA)(fC)(fU)(fA)(mU)(fU)(mG)(fA)(mA)(fU)(mC)(fU)#(mG)#(fC)#(mU)#(mA)#(mA)#(mU)#(fU) | 451 |
| P(mU)#(fA)#(mC)(fC)(fA)(fC)(mU)(fA)(mU)(fU)(mG)(fA)(mA)(fU)#(mC)#(fU)#(mG)#(mC)#(mU)#(mU)#(fU) | 452 |
| P(mU)#(fA)#(mC)(fC)(fU)(fU)(mC)(fU)(mA)(fA)(mU)(fC)(mU)(fU)#(mA)#(fA)#(mC)#(mA)#(mU)#(mU)#(fU) | 453 |
| P(mU)#(fA)#(mU)(fA)(fA)(fC)(mC)(fU)(mU)(fC)(mU)(fA)(mA)(fU)#(mC)#(fU)#(mU)#(mA)#(mA)#(mU)#(fU) | 454 |
| P(mU)#(fA)#(mU)(fA)(fU)(fA)(mA)(fC)(mC)(fU)(mU)(fC)(mU)(fA)#(mA)#(fU)#(mC)#(mU)#(mU)#(mU)#(fU) | 455 |
| P(mU)#(fU)#(mU)(fG)(fA)(fU)(mU)(fG)(mU)(fA)(mA)(fU)(mA)(fU)#(mA)#(fA)#(mC)#(mC)#(mU)#(mU)#(fU) | 456 |
| P(mU)#(fA)#(mA)(fU)(fU)(fG)(mA)(fU)(mU)(fG)(mU)(fA)(mA)(fU)#(mA)#(fU)#(mA)#(mA)#(mC)#(mU)#(fU) | 457 |
| P(mU)#(fU)#(mA)(fA)(fA)(fU)(mU)(fG)(mA)(fU)(mU)(fG)(mU)(fA)#(mA)#(fU)#(mA)#(mU)#(mA)#(mU)#(fU) | 458 |
| P(mU)#(fA)#(mC)(fA)(fU)(fA)(mA)(fA)(mU)(fU)(mG)(fA)(mU)(fU)#(mG)#(fU)#(mA)#(mA)#(mU)#(mU)#(fU) | 459 |
| P(mU)#(fU)#(mA)(fC)(fA)(fU)(mA)(fA)(mA)(fU)(mU)(fG)(mA)(fU)#(mU)#(fG)#(mU)#(mA)#(mA)#(mU)#(fU) | 460 |
| P(mU)#(fU)#(mG)(fA)(fA)(fU)(mG)(fU)(mU)(fG)(mA)(fU)(mA)(fA)#(mU)#(fG)#(mU)#(mA)#(mA)#(mU)#(fU) | 461 |
| P(mU)#(fC)#(mA)(fU)(fG)(fU)(mU)(fG)(mG)(fU)(mA)(fU)(mG)(fU)#(mU)#(fU)#(mA)#(mG)#(mU)#(mU)#(fU) | 462 |
| P(mU)#(fU)#(mA)(fC)(fA)(fU)(mG)(fU)(mU)(fG)(mG)(fU)(mA)(fU)#(mG)#(fU)#(mU)#(mU)#(mA)#(mU)#(fU) | 463 |
| P(mU)#(fU)#(mC)(fU)(fU)(fU)(mA)(fG)(mA)(fU)(mA)(fC)(mA)(fU)#(mG)#(fU)#(mU)#(mG)#(mG)#(mU)#(fU) | 464 |
| P(mU)#(fG)#(mA)(fG)(fA)(fU)(mA)(fC)(mC)(fA)(mU)(fA)(mU)(fU)#(mU)#(fG)#(mU)#(mA)#(mU)#(mU)#(fU) | 465 |
| P(mU)#(fC)#(mU)(fG)(fU)(fU)(mA)(fU)(mU)(fG)(mA)(fG)(mA)(fU)#(mA)#(fC)#(mC)#(mA)#(mU)#(mU)#(fU) | 466 |
| P(mU)#(fA)#(mG)(fU)(fA)(fG)(mC)(fU)(mG)(fU)(mU)(fA)(mU)(fU)#(mG)#(fA)#(mG)#(mA)#(mU)#(mU)#(fU) | 467 |
| P(mU)#(fA)#(mA)(fG)(fU)(fA)(mG)(fC)(mU)(fG)(mU)(fU)(mA)(fU)#(mU)#(fG)#(mA)#(mG)#(mA)#(mU)#(fU) | 468 |
| P(mU)#(fA)#(mA)(fA)(fG)(fU)(mA)(fG)(mC)(fU)(mG)(fU)(mU)(fA)#(mU)#(fU)#(mG)#(mA)#(mG)#(mU)#(fU) | 469 |
| P(mU)#(fU)#(mU)(fC)(fA)(fU)(mU)(fG)(mU)(fC)(mA)(fA)(mA)(fU)#(mU)#(fA)#(mU)#(mA)#(mA)#(mU)#(fU) | 470 |
| P(mU)#(fA)#(mA)(fA)(fU)(fG)(mU)(fA)(mU)(fU)(mC)(fA)(mU)(fU)#(mG)#(fU)#(mC)#(mA)#(mA)#(mU)#(fU) | 471 |
| P(mU)#(fG)#(mA)(fA)(fA)(fU)(mG)(fU)(mA)(fU)(mU)(fC)(mA)(fU)#(mU)#(fG)#(mU)#(mC)#(mA)#(mU)#(fU) | 472 |
| P(mU)#(fA)#(mG)(fA)(fA)(fA)(mU)(fG)(mU)(fA)(mU)(fU)(mC)(fA)#(mU)#(fU)#(mG)#(mU)#(mC)#(mU)#(fU) | 473 |
| P(mU)#(fA)#(mA)(fA)(fG)(fU)(mU)(fC)(mC)(fA)(mA)(fU)(mU)(fU)#(mA)#(fU)#(mA)#(mA)#(mA)#(mU)#(fU) | 474 |
| P(mU)#(fC)#(mA)(fA)(fA)(fG)(mU)(fU)(mC)(fC)(mA)(fA)(mU)(fU)#(mU)#(fA)#(mU)#(mA)#(mA)#(mU)#(fU) | 475 |
| P(mU)#(fA)#(mC)(fA)(fA)(fA)(mG)(fU)(mU)(fC)(mC)(fA)(mA)(fU)#(mU)#(fU)#(mA)#(mU)#(mA)#(mU)#(fU) | 476 |
| P(mU)#(fU)#(mU)(fU)(fG)(fA)(mU)(fA)(mA)(fA)(mC)(fA)(mA)(fA)#(mG)#(fU)#(mU)#(mC)#(mC)#(mU)#(fU) | 477 |
| P(mU)#(fU)#(mU)(fC)(fA)(fU)(mU)(fU)(mA)(fG)(mC)(fU)(mA)(fU)#(mG)#(fA)#(mG)#(mU)#(mA)#(mU)#(fU) | 478 |
| P(mU)#(fA)#(mA)(fU)(fA)(fA)(mU)(fU)(mU)(fC)(mA)(fU)(mU)(fU)#(mA)#(fG)#(mC)#(mU)#(mA)#(mU)#(fU) | 479 |
| P(mU)#(fG)#(mA)(fA)(fA)(fU)(mA)(fA)(mU)(fU)(mU)(fC)(mA)(fU)#(mU)#(fU)#(mA)#(mG)#(mC)#(mU)#(fU) | 480 |

m is2′-O-methyl nucleosides;
f is 2′-Fluoro nucleosides;
P is 5′-(E)-Vinylphosphonate;
# is phosphorothioate linkage;
TegChol is TEG linker + cholesterol

TABLE 5
Modified human MSTN specific siRNA

| | SEQ |
|---|---|
| | ID |
| | NO |
| Sense Strand | |
| (mU)#(mA)#(mA)(fU)(mG)(fC)(mA)(fU)(mG)(fU)(mA)(mC)(mU)(fU)#(mG)#(mA)-TegChol | 481 |
| (mG)#(mA)#(mA)(fU)(mA)(fG)(mA)(fA)(mG)(fC)(mC)(mA)(mU)(fU)#(mA)#(mA)-TegChol | 482 |
| (mU)#(mU)#(mA)(fA)(mG)(fA)(mU)(fA)(mC)(fA)(mA)(mA)(mU)(fC)#(mC)#(mA)-TegChol | 483 |
| (mU)#(mC)#(mA)(fG)(mC)(fA)(mA)(fA)(mG)(fA)(mU)(mG)(mU)(fU)#(mA)#(mA)-TegChol | 484 |
| (mG)#(mC)#(mU)(fU)(mC)(fU)(mU)(fU)(mA)(fA)(mA)(mU)(mU)(fU)#(mA)#(mA)-TegChol | 485 |
| (mA)#(mA)#(mA)(fG)(mG)(fC)(mC)(fC)(mA)(fA)(mC)(mU)(mA)(fU)#(mG)#(mA)-TegChol | 486 |
| (mA)#(mC)#(mU)(fC)(mC)(fU)(mA)(fC)(mA)(fA)(mC)(mA)(mG)(fU)#(mG)#(mA)-TegChol | 487 |
| (mC)#(mU)#(mA)(fC)(mA)(fA)(mC)(fA)(mG)(fU)(mG)(mU)(mU)(fU)#(mG)#(mA)-TegChol | 488 |
| (mU)#(mG)#(mA)(fA)(mG)(fA)(mC)(fA)(mG)(fU)(mG)(mU)(mU)(fG)#(mC)#(mA)-TegChol | 489 |
| (mC)#(mA)#(mG)(fA)(mA)(fU)(mC)(fA)(mC)(fG)(mA)(mU)(mG)(fC)#(mU)#(mA)-TegChol | 490 |
| (mA)#(mU)#(mG)(fG)(mG)(fA)(mU)(fU)(mG)(fG)(mA)(mU)(mU)(fA)#(mU)#(mA)-TegChol | 491 |
| (mG)#(mA)#(mU)(fU)(mG)(fG)(mA)(fU)(mU)(fA)(mU)(mC)(mG)(fC)#(mU)#(mA)-TegChol | 492 |
| (mG)#(mC)#(mU)(fC)(mU)(fG)(mG)(fA)(mG)(fA)(mG)(mU)(mG)(fU)#(mG)#(mA)-TegChol | 493 |
| (mG)#(mA)#(mG)(fA)(mG)(fU)(mG)(fU)(mG)(fA)(mA)(mU)(mU)(fU)#(mG)#(mA)-TegChol | 494 |
| (mA)#(mG)#(mA)(fG)(mU)(fG)(mU)(fG)(mA)(fA)(mU)(mU)(mU)(fG)#(mU)#(mA)-TegChol | 495 |
| (mC)#(mC)#(mA)(fA)(mU)(fU)(mA)(fA)(mU)(fA)(mU)(mG)(mC)(fU)#(mA)#(mA)-TegChol | 496 |
| (mC)#(mA)#(mA)(fU)(mU)(fA)(mA)(fU)(mA)(fU)(mG)(mC)(mU)(fA)#(mU)#(mA)-TegChol | 497 |
| (mU)#(mU)#(mA)(fA)(mU)(fA)(mU)(fG)(mC)(fU)(mA)(mU)(mA)(fU)#(mU)#(mA)-TegChol | 498 |
| (mG)#(mC)#(mA)(fA)(mA)(fG)(mA)(fA)(mC)(fA)(mA)(mA)(mU)(fA)#(mA)#(mA)-TegChol | 499 |
| (mC)#(mA)#(mA)(fA)(mG)(fA)(mA)(fC)(mA)(fA)(mA)(mU)(mA)(fA)#(mU)#(mA)-TegChol | 500 |
| (fA)#(mA)#(fG)(mA)(fA)(mC)(fA)(mA)(fA)(mU)(fA)(mA)(fU)#(mA)#(fA)-TegChol | 501 |
| (mC)#(mA)#(mU)(fA)(mA)(fG)(mC)(fU)(mA)(fC)(mA)(mG)(mU)(fA)#(mU)#(mA)-TegChol | 502 |
| (mA)#(mU)#(mA)(fU)(mA)(fU)(mG)(fC)(mA)(fA)(mU)(mG)(mG)(fU)#(mU)#(mA)-TegChol | 503 |
| (mU)#(mG)#(mA)(fU)(mU)(fU)(mC)(fC)(mA)(fG)(mA)(mG)(mU)(fU)#(mU)#(mA)-TegChol | 504 |
| (mA)#(mG)#(mC)(fU)(mA)(fG)(mA)(fA)(mG)(fG)(mA)(mG)(mA)(fU)#(mC)#(mA)-TegChol | 505 |
| (mG)#(mC)#(mU)(fA)(mG)(fA)(mA)(fG)(mG)(fA)(mG)(mA)(mU)(fC)#(mA)#(mA)-TegChol | 506 |
| (mU)#(mC)#(mA)(fA)(mA)(fU)(mU)(fA)(mC)(fA)(mU)(mU)(mU)(fA)#(mU)#(mA)-TegChol | 507 |
| (mA)#(mA)#(mU)(fU)(mA)(fC)(mA)(fU)(mU)(fU)(mA)(mU)(mG)(fU)#(mU)#(mA)-TegChol | 508 |
| (mU)#(mU)#(mA)(fC)(mA)(fU)(mU)(fU)(mA)(fU)(mG)(mU)(mU)(fC)#(mC)#(mA)-TegChol | 509 |
| (mA)#(mC)#(mA)(fU)(mU)(fU)(mA)(fU)(mG)(fU)(mU)(mC)(mC)(fU)#(mA)#(mA)-TegChol | 510 |
| (mA)#(mU)#(mA)(fU)(mA)(fU)(mU)(fA)(mC)(fA)(mA)(mC)(mA)(fU)#(mC)#(mA)-TegChol | 511 |
| (mA)#(mA)#(mA)(fU)(mG)(fA)(mA)(fA)(mG)(fC)(mG)(mA)(mU)(fU)#(mC)#(mA)-TegChol | 512 |
| (mG)#(mA)#(mA)(fU)(mU)(fA)(mA)(fA)(mG)(fG)(mA)(mG)(mU)(fA)#(mU)#(mA)-TegChol | 513 |
| (mG)#(mC)#(mU)(fU)(mU)(fA)(mA)(fA)(mG)(fU)(mC)(mU)(mA)(fU)#(mU)#(mA)-TegChol | 514 |
| (mA)#(mA)#(mG)(fU)(mC)(fU)(mA)(fU)(mU)(fU)(mC)(mU)(mU)(fU)#(mA)#(mA)-TegChol | 515 |
| (mA)#(mU)#(mU)(fU)(mC)(fU)(mU)(fU)(mA)(fA)(mA)(mG)(mU)(fU)#(mU)#(mA)-TegChol | 516 |
| (mU)#(mU)#(mA)(fU)(mA)(fU)(mA)(fC)(mC)(fA)(mU)(mC)(mA)(fU)#(mU)#(mA)-TegChol | 517 |
| (mA)#(mU)#(mA)(fU)(mA)(fU)(mU)(fU)(mC)(fU)(mG)(mC)(mA)(fU)#(mU)#(mA)-TegChol | 518 |
| (mU)#(mC)#(mA)(fU)(mC)(fC)(mU)(fU)(mA)(fA)(mA)(mC)(mA)(fC)#(mU)#(mA)-TegChol | 519 |
| (mA)#(mA)#(mC)(fA)(mC)(fU)(mU)(fG)(mA)(fA)(mU)(mU)(mU)(fA)#(mU)#(mA)-TegChol | 520 |
| (mA)#(mC)#(mU)(fU)(mG)(fA)(mA)(fU)(mU)(fU)(mA)(mU)(mA)(fU)#(mU)#(mA)-TegChol | 521 |
| (mG)#(mA)#(mA)(fU)(mU)(fU)(mA)(fU)(mA)(fU)(mU)(mG)(mU)(fA)#(mU)#(mA)-TegChol | 522 |
| (mA)#(mU)#(mG)(fG)(mU)(fG)(mC)(fA)(mG)(fC)(mA)(mU)(mA)(fU)#(mG)#(mA)-TegChol | 523 |
| (mG)#(mC)#(mA)(fU)(mA)(fU)(mG)(fC)(mA)(fA)(mU)(mU)(mU)(fC)#(mC)#(mA)-TegChol | 524 |
| (mA)#(mA)#(mA)(fC)(mU)(fG)(mC)(fA)(mU)(fU)(mA)(mU)(mC)(fU)#(mU)#(mA)-TegChol | 525 |
| (mA)#(mG)#(mA)(fA)(mG)(fU)(mA)(fU)(mG)(fU)(mA)(mA)(mU)(fU)#(mU)#(mA)-TegChol | 526 |
| (mC)#(mC)#(mU)(fU)(mU)(fG)(mC)(fA)(mA)(fC)(mA)(mC)(mU)(fG)#(mC)#(mA)-TegChol | 527 |
| (mG)#(mC)#(mA)(fA)(mC)(fA)(mC)(fU)(mG)(fC)(mA)(mG)(mU)(fU)#(mU)#(mA)-TegChol | 528 |
| (mG)#(mA)#(mA)(fA)(mU)(fG)(mA)(fU)(mC)(fG)(mA)(mC)(mU)(fC)#(mU)#(mA)-TegChol | 529 |
| (mA)#(mA)#(mA)(fU)(mG)(fA)(mU)(fC)(mG)(fA)(mC)(mU)(mC)(fU)#(mA)#(mA)-TegChol | 530 |
| (mG)#(mA)#(mC)(fU)(mC)(fU)(mA)(fU)(mC)(fA)(mA)(mU)(mA)(fU)#(mU)#(mA)-TegChol | 531 |
| (mC)#(mU)#(mA)(fU)(mC)(fA)(mA)(fU)(mA)(fU)(mU)(mG)(mU)(fA)#(mU)#(mA)-TegChol | 532 |
| (mG)#(mA)#(mC)(fU)(mG)(fA)(mA)(fA)(mC)(fA)(mA)(mU)(mG)(fC)#(mA)#(mA)-TegChol | 533 |
| (mA)#(mA)#(mA)(fC)(mA)(fA)(mU)(fG)(mC)(fA)(mU)(mU)(mU)(fA)#(mU)#(mA)-TegChol | 534 |
| (mA)#(mC)#(mA)(fA)(mU)(fG)(mC)(fA)(mU)(fU)(mU)(mA)(mU)(fA)#(mU)#(mA)-TegChol | 535 |
| (mC)#(mA)#(mA)(fU)(mG)(fC)(mA)(fU)(mU)(fU)(mA)(mU)(mA)(fU)#(mA)#(mA)-TegChol | 536 |
| (mU)#(mA)#(mA)(fU)(mA)(fU)(mG)(fU)(mA)(fU)(mA)(mC)(mA)(fA)#(mU)#(mA)-TegChol | 537 |
| (mU)#(mA)#(mU)(fG)(mU)(fA)(mU)(fA)(mC)(fA)(mA)(mU)(mA)(fU)#(mU)#(mA)-TegChol | 538 |
| (mU)#(mA)#(mU)(fA)(mC)(fA)(mA)(fU)(mA)(fU)(mU)(mG)(mU)(fU)#(mU)#(mA)-TegChol | 539 |
| (mU)#(mU)#(mG)(fU)(mA)(fA)(mA)(fU)(mA)(fA)(mG)(mU)(mG)(fU)#(mC)#(mA)-TegChol | 540 |
| (mG)#(mU)#(mA)(fA)(mA)(fU)(mA)(fA)(mG)(fU)(mG)(mU)(mC)(fU)#(mC)#(mA)-TegChol | 541 |
| (mU)#(mA)#(mA)(fA)(mU)(fA)(mA)(fG)(mU)(fG)(mU)(mC)(mU)(fC)#(mC)#(mA)-TegChol | 542 |
| (mA)#(mA)#(mA)(fU)(mA)(fA)(mG)(fU)(mG)(fU)(mC)(mU)(mC)(fC)#(mU)#(mA)-TegChol | 543 |
| (mA)#(mC)#(mA)(fC)(mU)(fA)(mA)(fG)(mG)(fA)(mC)(mA)(mU)(fU)#(mU)#(mA)-TegChol | 544 |
| (mA)#(mC)#(mU)(fA)(mA)(fG)(mG)(fA)(mC)(fA)(mU)(mU)(mU)(fC)#(mA)#(mA)-TegChol | 545 |
| (mA)#(mC)#(mA)(fU)(mU)(fU)(mC)(fA)(mA)(fA)(mU)(mU)(mA)(fA)#(mG)#(mA)-TegChol | 546 |
| (mG)#(mC)#(mA)(fG)(mA)(fU)(mU)(fA)(mA)(fA)(mU)(mA)(mG)(fU)#(mG)#(mA)-TegChol | 547 |
| (mC)#(mA)#(mG)(fA)(mU)(fU)(mA)(fA)(mA)(fU)(mA)(mG)(mU)(fG)#(mG)#(mA)-TegChol | 548 |
| (mA)#(mG)#(mA)(fU)(mU)(fA)(mA)(fA)(mU)(fA)(mG)(mU)(mG)(fG)#(mU)#(mA)-TegChol | 549 |
| (mU)#(mA)#(mA)(fU)(mG)(fA)(mU)(fU)(mA)(fG)(mA)(mU)(mG)(fG)#(mU)#(mA)-TegChol | 550 |
| (mG)#(mA)#(mU)(fG)(mG)(fU)(mU)(fA)(mU)(fA)(mU)(mU)(mA)(fC)#(mA)#(mA)-TegChol | 551 |
| (mU)#(mU)#(mA)(fU)(mA)(fU)(mU)(fA)(mC)(fA)(mA)(mU)(mC)(fA)#(mU)#(mA)-TegChol | 552 |
| (mA)#(mC)#(mA)(fU)(mG)(fA)(mU)(fU)(mA)(fA)(mC)(mA)(mU)(fU)#(mC)#(mA)-TegChol | 553 |
| (mC)#(mA)#(mU)(fG)(mA)(fU)(mU)(fA)(mA)(fC)(mA)(mU)(mU)(fC)#(mA)#(mA)-TegChol | 554 |
| (mG)#(mU)#(mA)(fU)(mA)(fA)(mA)(fG)(mU)(fG)(mA)(mA)(mU)(fU)#(mU)#(mA)-TegChol | 555 |
| (mA)#(mA)#(mA)(fU)(mU)(fU)(mC)(fA)(mA)(fU)(mG)(mG)(mU)(fU)#(mU)#(mA)-TegChol | 556 |
| (mA)#(mU)#(mU)(fU)(mC)(fA)(mA)(fU)(mG)(fG)(mU)(mU)(mU)(fA)#(mC)#(mA)-TegChol | 557 |
| (mU)#(mU)#(mG)(fU)(mG)(fU)(mU)(fU)(mA)(fA)(mA)(mU)(mC)(fU)#(mC)#(mA)-TegChol | 558 |
| (mU)#(mU)#(mA)(fA)(mA)(fU)(mC)(fU)(mC)(fA)(mA)(mC)(mG)(fU)#(mU)#(mA)-TegChol | 559 |
| (mA)#(mA)#(mA)(fU)(mC)(fU)(mC)(fA)(mA)(fC)(mG)(mU)(mU)(fC)#(mC)#(mA)-TegChol | 560 |
| (mA)#(mA)#(mC)(fU)(mG)(fA)(mU)(fG)(mC)(fU)(mA)(mU)(mC)(fU)#(mC)#(mA)-TegChol | 561 |
| (mU)#(mC)#(mA)(fA)(mC)(fA)(mA)(fU)(mA)(fA)(mC)(mU)(mG)(fU)#(mU)#(mA)-TegChol | 562 |
| (mC)#(mA)#(mA)(fC)(mA)(fA)(mU)(fA)(mA)(fC)(mU)(mG)(mU)(fU)#(mA)#(mA)-TegChol | 563 |
| (mU)#(mA)#(mA)(fU)(mU)(fU)(mG)(fA)(mU)(fA)(mA)(mU)(mG)(fA)#(mA)#(mA)-TegChol | 564 |
| (mU)#(mA)#(mA)(fU)(mG)(fA)(mA)(fU)(mA)(fU)(mA)(mU)(mU)(fU)#(mC)#(mA)-TegChol | 565 |
| (mA)#(mA)#(mU)(fG)(mA)(fA)(mU)(fA)(mU)(fA)(mU)(mU)(mU)(fC)#(mU)#(mA)-TegChol | 566 |
| (mG)#(mA)#(mA)(fU)(mA)(fU)(mA)(fU)(mU)(fU)(mC)(mU)(mG)(fC)#(mA)#(mA)-TegChol | 567 |
| (mC)#(mU)#(mG)(fC)(mA)(fU)(mU)(fU)(mA)(fU)(mU)(mU)(mA)(fC)#(mU)#(mA)-TegChol | 568 |
| (mA)#(mU)#(mU)(fU)(mA)(fU)(mU)(fU)(mA)(fC)(mU)(mU)(mC)(fU)#(mG)#(mA)-TegChol | 569 |
| (mU)#(mA)#(mA)(fU)(mC)(fA)(mA)(fA)(mU)(fU)(mU)(mA)(mU)(fU)#(mG)#(mA)-TegChol | 570 |
| (mA)#(mC)#(mU)(fA)(mA)(fA)(mU)(fG)(mA)(fA)(mA)(mU)(mU)(fA)#(mU)#(mA)-TegChol | 571 |
| (mC)#(mU)#(mA)(fA)(mA)(fU)(mG)(fA)(mA)(fA)(mU)(mU)(mA)(fU)#(mU)#(mA)-TegChol | 572 |
| (mC)#(mU)#(mU)(fA)(mC)(fA)(mU)(fC)(mU)(fA)(mA)(mU)(mU)(fU)#(mG)#(mA)-TegChol | 573 |
| (mA)#(mC)#(mA)(fU)(mC)(fU)(mA)(fA)(mU)(fU)(mU)(mG)(mU)(fA)#(mG)#(mA)-TegChol | 574 |
| (mU)#(mA)#(mG)(fA)(mA)(fA)(mC)(fA)(mG)(fU)(mA)(mU)(mA)(fA)#(mG)#(mA)-TegChol | 575 |
| (mG)#(mA)#(mA)(fA)(mC)(fA)(mG)(fU)(mA)(fU)(mA)(mA)(mG)(fU)#(mU)#(mA)-TegChol | 576 |
| (mA)#(mC)#(mA)(fG)(mU)(fA)(mU)(fA)(mA)(fG)(mU)(mU)(mA)(fU)#(mA)#(mA)-TegChol | 577 |
| Antisense Strand | |
| P(mU)#(fC)#(mA)(fA)(fG)(fU)(mA)(fC)(mA)(fU)(mG)(fC)(mA)(fU)#(mU)#(fA)#(mC)#(mA)#(mC)#(mU)#(fU) | 578 |
| P(mU)#(fU)#(mA)(fA)(fU)(fG)(mG)(fC)(mU)(fU)(mC)(fU)(mA)(fU)#(mU)#(fC)#(mU)#(mU)#(mG)#(mU)#(fU) | 579 |
| P(mU)#(fG)#(mG)(fA)(fU)(fU)(mU)(fG)(mU)(fA)(mU)(fC)(mU)(fU)#(mA)#(fA)#(mU)#(mG)#(mG)#(mU)#(fU) | 580 |
| P(mU)#(fU)#(mA)(fA)(fC)(fA)(mU)(fC)(mU)(fU)(mU)(fG)(mC)(fU)#(mG)#(fA)#(mU)#(mG)#(mU)#(mU)#(fU) | 581 |
| P(mU)#(fU)#(mA)(fA)(fA)(fU)(mU)(fU)(mA)(fA)(mA)(fG)(mA)(fA)#(mG)#(fC)#(mA)#(mA)#(mC)#(mU)#(fU) | 582 |
| P(mU)#(fC)#(mA)(fU)(fA)(fG)(mU)(fU)(mG)(fG)(mG)(fC)(mC)(fU)#(mU)#(fU)#(mA)#(mC)#(mU)#(mU)#(fU) | 583 |
| P(mU)#(fC)#(mA)(fC)(fU)(fG)(mU)(fU)(mG)(fU)(mA)(fG)(mG)(fA)#(mG)#(fU)#(mC)#(mU)#(mC)#(mU)#(fU) | 584 |
| P(mU)#(fC)#(mA)(fA)(fA)(fC)(mA)(fC)(mU)(fG)(mU)(fU)(mG)(fU)#(mA)#(fG)#(mG)#(mA)#(mG)#(mU)#(fU) | 585 |
| P(mU)#(fG)#(mC)(fA)(fA)(fC)(mA)(fC)(mU)(fG)(mU)(fC)(mU)(fU)#(mC)#(fA)#(mC)#(mA)#(mU)#(mU)#(fU) | 586 |
| P(mU)#(fA)#(mG)(fC)(fA)(fU)(mC)(fG)(mU)(fG)(mA)(fU)(mU)(fC)#(mU)#(fG)#(mU)#(mU)#(mG)#(mU)#(fU) | 587 |
| P(mU)#(fA)#(mU)(fA)(fA)(fU)(mC)(fC)(mA)(fA)(mU)(fC)(mC)(fC)#(mA)#(fU)#(mC)#(mC)#(mA)#(mU)#(fU) | 588 |
| P(mU)#(fA)#(mG)(fC)(fG)(fA)(mU)(fA)(mA)(fU)(mC)(fC)(mA)(fA)#(mU)#(fC)#(mC)#(mC)#(mA)#(mU)#(fU) | 589 |
| P(mU)#(fC)#(mA)(fC)(fA)(fC)(mU)(fC)(mU)(fC)(mC)(fA)(mG)(fA)#(mG)#(fC)#(mA)#(mG)#(mU)#(mU)#(fU) | 590 |
| P(mU)#(fC)#(mA)(fA)(fA)(fU)(mU)(fC)(mA)(fC)(mA)(fC)(mU)(fC)#(mU)#(fC)#(mC)#(mA)#(mG)#(mU)#(fU) | 591 |
| P(mU)#(fA)#(mC)(fA)(fA)(fA)(mU)(fU)(mC)(fA)(mC)(fA)(mC)(fU)#(mC)#(fU)#(mC)#(mC)#(mA)#(mU)#(fU) | 592 |
| P(mU)#(fU)#(mA)(fG)(fC)(fA)(mU)(fA)(mU)(fU)(mA)(fA)(mU)(fU)#(mG)#(fG)#(mA)#(mG)#(mA)#(mU)#(fU) | 593 |
| P(mU)#(fA)#(mU)(fA)(fG)(fC)(mA)(fU)(mA)(fU)(mU)(fA)(mA)(fU)#(mU)#(fG)#(mG)#(mA)#(mG)#(mU)#(fU) | 594 |
| P(mU)#(fA)#(mA)(fU)(fA)(fU)(mA)(fG)(mC)(fA)(mU)(fA)(mU)(fU)#(mA)#(fA)#(mU)#(mU)#(mG)#(mU)#(fU) | 595 |
| P(mU)#(fU)#(mU)(fA)(fU)(fU)(mU)(fG)(mU)(fU)(mC)(fU)(mU)(fU)#(mG)#(fC)#(mC)#(mA)#(mU)#(mU)#(fU) | 596 |
| P(mU)#(fA)#(mU)(fU)(fA)(fU)(mU)(fU)(mG)(fU)(mU)(fC)(mU)(fU)#(mU)#(fG)#(mC)#(mC)#(mA)#(mU)#(fU) | 597 |
| P(mU)#(fU)#(mA)(fU)(mU)(fA)(mU)(fU)(mU)(fG)(mU)(fU)(mC)#(fU)#(mU)#(fU)#(mG)#(fC)#(mC)#(fU) | 598 |
| P(mU)#(fA)#(mU)(fA)(fC)(fU)(mG)(fU)(mA)(fG)(mC)(fU)(mU)(fA)#(mU)#(fG)#(mC)#(mU)#(mU)#(mU)#(fU) | 599 |
| P(mU)#(fA)#(mA)(fC)(fC)(fA)(mU)(fU)(mG)(fC)(mA)(fU)(mA)(fU)#(mA)#(fU)#(mU)#(mC)#(mC)#(mU)#(fU) | 600 |
| P(mU)#(fA)#(mA)(fA)(fC)(fU)(mC)(fU)(mG)(fG)(mA)(fA)(mA)(fU)#(mC)#(fA)#(mU)#(mA)#(mA)#(mU)#(fU) | 601 |
| P(mU)#(fG)#(mA)(fU)(fC)(fU)(mC)(fC)(mU)(fU)(mC)(fU)(mA)(fG)#(mC)#(fU)#(mC)#(mA)#(mA)#(mU)#(fU) | 602 |
| P(mU)#(fU)#(mG)(fA)(fU)(fC)(mU)(fC)(mC)(fU)(mU)(fC)(mU)(fA)#(mG)#(fC)#(mU)#(mC)#(mA)#(mU)#(fU) | 603 |
| P(mU)#(fA)#(mU)(fA)(fA)(fA)(mU)(fG)(mU)(fA)(mA)(fU)(mU)(fU)#(mG)#(fA)#(mU)#(mC)#(mU)#(mU)#(fU) | 604 |
| P(mU)#(fA)#(mA)(fC)(fA)(fU)(mA)(fA)(mA)(fU)(mG)(fU)(mA)(fA)#(mU)#(fU)#(mU)#(mG)#(mA)#(mU)#(fU) | 605 |
| P(mU)#(fG)#(mG)(fA)(fA)(fC)(mA)(fU)(mA)(fA)(mA)(fU)(mG)(fU)#(mA)#(fA)#(mU)#(mU)#(mU)#(mU)#(fU) | 606 |
| P(mU)#(fU)#(mA)(fG)(fG)(fA)(mA)(fC)(mA)(fU)(mA)(fA)(mA)(fU)#(mG)#(fU)#(mA)#(mA)#(mU)#(mU)#(fU) | 607 |
| P(mU)#(fG)#(mA)(fU)(fG)(fU)(mU)(fG)(mU)(fA)(mA)(fU)(mA)(fU)#(mA)#(fU)#(mA)#(mG)#(mG)#(mU)#(fU) | 608 |
| P(mU)#(fG)#(mA)(fA)(fU)(fC)(mG)(fC)(mU)(fU)(mU)(fC)(mA)(fU)#(mU)#(fU)#(mC)#(mC)#(mU)#(mU)#(fU) | 609 |
| P(mU)#(fA)#(mU)(fA)(fC)(fU)(mC)(fC)(mU)(fU)(mU)(fA)(mA)(fU)#(mU)#(fC)#(mA)#(mU)#(mC)#(mU)#(fU) | 610 |
| P(mU)#(fA)#(mA)(fU)(fA)(fG)(mA)(fC)(mU)(fU)(mU)(fA)(mA)(fA)#(mG)#(fC)#(mA)#(mU)#(mA)#(mU)#(fU) | 611 |
| P(mU)#(fU)#(mA)(fA)(fA)(fG)(mA)(fA)(mA)(fU)(mA)(fG)(mA)(fC)#(mU)#(fU)#(mU)#(mA)#(mA)#(mU)#(fU) | 612 |
| P(mU)#(fA)#(mA)(fA)(fC)(fU)(mU)(fU)(mA)(fA)(mA)(fG)(mA)(fA)#(mA)#(fU)#(mA)#(mG)#(mA)#(mU)#(fU) | 613 |
| P(mU)#(fA)#(mA)(fU)(fG)(fA)(mU)(fG)(mG)(fU)(mA)(fU)(mA)(fU)#(mA)#(fA)#(mC)#(mA)#(mA)#(mU)#(fU) | 614 |
| P(mU)#(fA)#(mA)(fU)(fG)(fC)(mA)(fG)(mA)(fA)(mA)(fU)(mA)(fU)#(mA)#(fU)#(mU)#(mC)#(mA)#(mU)#(fU) | 615 |
| P(mU)#(fA)#(mG)(fU)(fG)(fU)(mU)(fU)(mA)(fA)(mG)(fG)(mA)(fU)#(mG)#(fA)#(mU)#(mU)#(mC)#(mU)#(fU) | 616 |
| P(mU)#(fA)#(mU)(fA)(fA)(fA)(mU)(fU)(mC)(fA)(mA)(fG)(mU)(fG)#(mU)#(fU)#(mU)#(mA)#(mA)#(mU)#(fU) | 617 |
| P(mU)#(fA)#(mA)(fU)(fA)(fU)(mA)(fA)(mA)(fU)(mU)(fC)(mA)(fA)#(mG)#(fU)#(mG)#(mU)#(mU)#(mU)#(fU) | 618 |
| P(mU)#(fA)#(mU)(fA)(fC)(fA)(mA)(fU)(mA)(fU)(mA)(fA)(mA)(fU)#(mU)#(fC)#(mA)#(mA)#(mG)#(mU)#(fU) | 619 |
| P(mU)#(fC)#(mA)(fU)(fA)(fU)(mG)(fC)(mU)(fG)(mC)(fA)(mC)(fC)#(mA)#(fU)#(mC)#(mC)#(mC)#(mU)#(fU) | 620 |
| P(mU)#(fG)#(mG)(fA)(fA)(fA)(mU)(fU)(mG)(fC)(mA)(fU)(mA)(fU)#(mG)#(fC)#(mU)#(mG)#(mC)#(mU)#(fU) | 621 |
| P(mU)#(fA)#(mA)(fG)(fA)(fU)(mA)(fA)(mU)(fG)(mC)(fA)(mG)(fU)#(mU)#(fU)#(mC)#(mU)#(mC)#(mU)#(fU) | 622 |
| P(mU)#(fA)#(mA)(fA)(fU)(fU)(mA)(fC)(mA)(fU)(mA)(fC)(mU)(fU)#(mC)#(fU)#(mC)#(mC)#(mA)#(mU)#(fU) | 623 |
| P(mU)#(fG)#(mC)(fA)(fG)(fU)(mG)(fU)(mU)(fG)(mC)(fA)(mA)(fA)#(mG)#(fG)#(mC)#(mA)#(mG)#(mU)#(fU) | 624 |
| P(mU)#(fA)#(mA)(fA)(fC)(fU)(mG)(fC)(mA)(fG)(mU)(fG)(mU)(fU)#(mG)#(fC)#(mA)#(mA)#(mA)#(mU)#(fU) | 625 |
| P(mU)#(fA)#(mG)(fA)(fG)(fU)(mC)(fG)(mA)(fU)(mC)(fA)(mU)(fU)#(mU)#(fC)#(mU)#(mA)#(mU)#(mU)#(fU) | 626 |
| P(mU)#(fU)#(mA)(fG)(fA)(fG)(mU)(fC)(mG)(fA)(mU)(fC)(mA)(fU)#(mU)#(fU)#(mC)#(mU)#(mA)#(mU)#(fU) | 627 |
| P(mU)#(fA)#(mA)(fU)(fA)(fU)(mU)(fG)(mA)(fU)(mA)(fG)(mA)(fG)#(mU)#(fC)#(mG)#(mA)#(mU)#(mU)#(fU) | 628 |
| P(mU)#(fA)#(mU)(fA)(fC)(fA)(mA)(fU)(mA)(fU)(mU)(fG)(mA)(fU)#(mA)#(fG)#(mA)#(mG)#(mU)#(mU)#(fU) | 629 |
| P(mU)#(fU)#(mG)(fC)(fA)(fU)(mU)(fG)(mU)(fU)(mU)(fC)(mA)(fG)#(mU)#(fC)#(mU)#(mU)#(mU)#(mU)#(fU) | 630 |
| P(mU)#(fA)#(mU)(fA)(fA)(fA)(mU)(fG)(mC)(fA)(mU)(fU)(mG)(fU)#(mU)#(fU)#(mC)#(mA)#(mG)#(mU)#(fU) | 631 |
| P(mU)#(fA)#(mU)(fA)(fU)(fA)(mA)(fA)(mU)(fG)(mC)(fA)(mU)(fU)#(mG)#(fU)#(mU)#(mU)#(mC)#(mU)#(fU) | 632 |
| P(mU)#(fU)#(mA)(fU)(fA)(fU)(mA)(fA)(mA)(fU)(mG)(fC)(mA)(fU)#(mU)#(fG)#(mU)#(mU)#(mU)#(mU)#(fU) | 633 |
| P(mU)#(fA)#(mU)(fU)(fG)(fU)(mA)(fU)(mA)(fC)(mA)(fU)(mA)(fU)#(mU)#(fA)#(mU)#(mA)#(mU)#(mU)#(fU) | 634 |
| P(mU)#(fA)#(mA)(fU)(fA)(fU)(mU)(fG)(mU)(fA)(mU)(fA)(mC)(fA)#(mU)#(fA)#(mU)#(mU)#(mA)#(mU)#(fU) | 635 |
| P(mU)#(fA)#(mA)(fA)(fC)(fA)(mA)(fU)(mA)(fU)(mU)(fG)(mU)(fA)#(mU)#(fA)#(mC)#(mA)#(mU)#(mU)#(fU) | 636 |
| P(mU)#(fG)#(mA)(fC)(fA)(fC)(mU)(fU)(mA)(fU)(mU)(fU)(mA)(fC)#(mA)#(fA)#(mA)#(mA)#(mC)#(mU)#(fU) | 637 |
| P(mU)#(fG)#(mA)(fG)(fA)(fC)(mA)(fC)(mU)(fU)(mA)(fU)(mU)(fU)#(mA)#(fC)#(mA)#(mA)#(mA)#(mU)#(fU) | 638 |
| P(mU)#(fG)#(mG)(fA)(fG)(fA)(mC)(fA)(mC)(fU)(mU)(fA)(mU)(fU)#(mU)#(fA)#(mC)#(mA)#(mA)#(mU)#(fU) | 639 |
| P(mU)#(fA)#(mG)(fG)(fA)(fG)(mA)(fC)(mA)(fC)(mU)(fU)(mA)(fU)#(mU)#(fU)#(mA)#(mC)#(mA)#(mU)#(fU) | 640 |
| P(mU)#(fA)#(mA)(fA)(fU)(fG)(mU)(fC)(mC)(fU)(mU)(fA)(mG)(fU)#(mG)#(fU)#(mA)#(mA)#(mA)#(mU)#(fU) | 641 |
| P(mU)#(fU)#(mG)(fA)(fA)(fA)(mU)(fG)(mU)(fC)(mC)(fU)(mU)(fA)#(mG)#(fU)#(mG)#(mU)#(mA)#(mU)#(fU) | 642 |
| P(mU)#(fC)#(mU)(fU)(fA)(fA)(mU)(fU)(mU)(fG)(mA)(fA)(mA)(fU)#(mG)#(fU)#(mC)#(mC)#(mU)#(mU)#(fU) | 643 |
| P(mU)#(fC)#(mA)(fC)(fU)(fA)(mU)(fU)(mU)(fA)(mA)(fU)(mC)(fU)#(mG)#(fC)#(mU)#(mA)#(mA)#(mU)#(fU) | 644 |
| P(mU)#(fC)#(mC)(fA)(fC)(fU)(mA)(fU)(mU)(fU)(mA)(fA)(mU)(fC)#(mU)#(fG)#(mC)#(mU)#(mA)#(mU)#(fU) | 645 |
| P(mU)#(fA)#(mC)(fC)(fA)(fC)(mU)(fA)(mU)(fU)(mU)(fA)(mA)(fU)#(mC)#(fU)#(mG)#(mC)#(mU)#(mU)#(fU) | 646 |
| P(mU)#(fA)#(mC)(fC)(fA)(fU)(mC)(fU)(mA)(fA)(mU)(fC)(mA)(fU)#(mU)#(fA)#(mA)#(mC)#(mA)#(mU)#(fU) | 647 |
| P(mU)#(fU)#(mG)(fU)(fA)(fA)(mU)(fA)(mU)(fA)(mA)(fC)(mC)(fA)#(mU)#(fC)#(mU)#(mA)#(mA)#(mU)#(fU) | 648 |
| P(mU)#(fA)#(mU)(fG)(fA)(fU)(mU)(fG)(mU)(fA)(mA)(fU)(mA)(fU)#(mA)#(fA)#(mC)#(mC)#(mA)#(mU)#(fU) | 649 |
| P(mU)#(fG)#(mA)(fA)(fU)(fG)(mU)(fU)(mA)(fA)(mU)(fC)(mA)(fU)#(mG)#(fU)#(mA)#(mA)#(mA)#(mU)#(fU) | 650 |
| P(mU)#(fU)#(mG)(fA)(fA)(fU)(mG)(fU)(mU)(fA)(mA)(fU)(mC)(fA)#(mU)#(fG)#(mU)#(mA)#(mA)#(mU)#(fU) | 651 |
| P(mU)#(fA)#(mA)(fA)(fU)(fU)(mC)(fA)(mC)(fU)(mU)(fU)(mA)(fU)#(mA)#(fC)#(mA)#(mG)#(mC)#(mU)#(fU) | 652 |
| P(mU)#(fA)#(mA)(fA)(fC)(fC)(mA)(fU)(mU)(fG)(mA)(fA)(mA)(fU)#(mU)#(fU)#(mC)#(mA)#(mA)#(mU)#(fU) | 653 |
| P(mU)#(fG)#(mU)(fA)(fA)(fA)(mC)(fC)(mA)(fU)(mU)(fG)(mA)(fA)#(mA)#(fU)#(mU)#(mU)#(mC)#(mU)#(fU) | 654 |
| P(mU)#(fG)#(mA)(fG)(fA)(fU)(mU)(fU)(mA)(fA)(mA)(fC)(mA)(fC)#(mA)#(fA)#(mU)#(mG)#(mA)#(mU)#(fU) | 655 |
| P(mU)#(fA)#(mA)(fC)(fG)(fU)(mU)(fG)(mA)(fG)(mA)(fU)(mU)(fU)#(mA)#(fA)#(mA)#(mC)#(mA)#(mU)#(fU) | 656 |
| P(mU)#(fG)#(mG)(fA)(fA)(fC)(mG)(fU)(mU)(fG)(mA)(fG)(mA)(fU)#(mU)#(fU)#(mA)#(mA)#(mA)#(mU)#(fU) | 657 |
| P(mU)#(fG)#(mA)(fG)(fA)(fU)(mA)(fG)(mC)(fA)(mU)(fC)(mA)(fG)#(mU)#(fU)#(mU)#(mA)#(mU)#(mU)#(fU) | 658 |
| P(mU)#(fA)#(mA)(fC)(fA)(fG)(mU)(fU)(mA)(fU)(mU)(fG)(mU)(fU)#(mG)#(fA)#(mG)#(mA)#(mU)#(mU)#(fU) | 659 |
| P(mU)#(fU)#(mA)(fA)(fC)(fA)(mG)(fU)(mU)(fA)(mU)(fU)(mG)(fU)#(mU)#(fG)#(mA)#(mG)#(mA)#(mU)#(fU) | 660 |
| P(mU)#(fU)#(mU)(fC)(fA)(fU)(mU)(fA)(mU)(fC)(mA)(fA)(mA)(fU)#(mU)#(fA)#(mU)#(mA)#(mA)#(mU)#(fU) | 661 |
| P(mU)#(fG)#(mA)(fA)(fA)(fU)(mA)(fU)(mA)(fU)(mU)(fC)(mA)(fU)#(mU)#(fA)#(mU)#(mC)#(mA)#(mU)#(fU) | 662 |
| P(mU)#(fA)#(mG)(fA)(fA)(fA)(mU)(fA)(mU)(fA)(mU)(fU)(mC)(fA)#(mU)#(fU)#(mA)#(mU)#(mC)#(mU)#(fU) | 663 |
| P(mU)#(fU)#(mG)(fC)(fA)(fG)(mA)(fA)(mA)(fU)(mA)(fU)(mA)(fU)#(mU)#(fC)#(mA)#(mU)#(mU)#(mU)#(fU) | 664 |
| P(mU)#(fA)#(mG)(fU)(fA)(fA)(mA)(fU)(mA)(fA)(mA)(fU)(mG)(fC)#(mA)#(fG)#(mA)#(mA)#(mA)#(mU)#(fU) | 665 |
| P(mU)#(fC)#(mA)(fG)(fA)(fA)(mG)(fU)(mA)(fA)(mA)(fU)(mA)(fA)#(mA)#(fU)#(mG)#(mC)#(mA)#(mU)#(fU) | 666 |
| P(mU)#(fC)#(mA)(fA)(fU)(fA)(mA)(fA)(mU)(fU)(mU)(fG)(mA)(fU)#(mU)#(fA)#(mA)#(mC)#(mA)#(mU)#(fU) | 667 |
| P(mU)#(fA)#(mU)(fA)(fA)(fU)(mU)(fU)(mC)(fA)(mU)(fU)(mU)(fA)#(mG)#(fU)#(mC)#(mA)#(mU)#(mU)#(fU) | 668 |
| P(mU)#(fA)#(mA)(fU)(fA)(fA)(mU)(fU)(mU)(fC)(mA)(fU)(mU)(fU)#(mA)#(fG)#(mU)#(mC)#(mA)#(mU)#(fU) | 669 |
| P(mU)#(fC)#(mA)(fA)(fA)(fU)(mU)(fA)(mG)(fA)(mU)(fG)(mU)(fA)#(mA)#(fG)#(mA)#(mA)#(mA)#(mU)#(fU) | 670 |
| P(mU)#(fC)#(mU)(fA)(fC)(fA)(mA)(fA)(mU)(fU)(mA)(fG)(mA)(fU)#(mG)#(fU)#(mA)#(mA)#(mG)#(mU)#(fU) | 671 |
| P(mU)#(fC)#(mU)(fU)(fA)(fU)(mA)(fC)(mU)(fG)(mU)(fU)(mU)(fC)#(mU)#(fA)#(mC)#(mA)#(mA)#(mU)#(fU) | 672 |
| P(mU)#(fA)#(mA)(fC)(fU)(fU)(mA)(fU)(mA)(fC)(mU)(fG)(mU)(fU)#(mU)#(fC)#(mU)#(mA)#(mC)#(mU)#(fU) | 673 |
| P(mU)#(fU)#(mA)(fU)(fA)(fA)(mC)(fU)(mU)(fA)(mU)(fA)(mC)(fU)#(mG)#(fU)#(mU)#(mU)#(mC)#(mU)#(fU) | 674 |

m is2′-O-methyl nucleosides;
f is 2′-Fluoro nucleosides;
P is 5′-(E)-Vinylphosphonate;
# is phosphorothioate linkage;
TegChol is TEG linker + cholesterol

In some embodiments, a human MSTN specific siRNA comprises an anti-sense strand that comprises the nucleic acid sequence with chemical modification pattern set forth in Table 5. In some embodiments, a human MSTN specific siRNA comprises an anti-sense strand that comprises the nucleic acid sequence with chemical modification pattern as set forth in SEQ ID NOs: 578-674. In some embodiments, a human MSTN specific siRNA comprises a sense strand that comprises the nucleic acid sequence with chemical modification pattern set forth in Table 5. In some embodiments, a human MSTN specific siRNA comprises a sense strand that comprises the nucleic acid sequence with chemical modification pattern as set forth in SEQ ID NOs: 285-372.

In some embodiments, a mouse Mstn specific siRNA comprises an anti-sense strand that comprises the nucleic acid sequence with chemical modification pattern set forth in Table 4. In some embodiments, a mouse Mstn specific siRNA comprises an anti-sense strand that comprises the nucleic acid sequence with chemical modification pattern as set forth in SEQ ID NOs: 373-480. In some embodiments, a mouse Mstn specific siRNA comprises a sense strand that comprises the nucleic acid sequence with chemical modification pattern set forth in Table 4. In some embodiments, a mouse Mstn specific siRNA comprises a sense strand that comprises the nucleic acid sequence with chemical modification pattern as set forth in SEQ ID NOs: 481-577.

iv. Bispecific siRNA

In some aspects, the present disclosure provides bi-specific nucleic acid molecules (e.g., bispecific siRNAs) comprising one or more siRNAs described herein. In some embodiments, bi-specific nucleic acid molecule (e.g., bispecific siRNA) comprises: (i) a first siRNA targeting a first RNA transcript selected from ACVR1^{R206H}, Inhba, IL-1β, TNF, and myostatin (Mstn); linked to (ii) a second siRNA targeting a second RNA transcript selected from ACVR1^{R206H}, Inhba, IL-1β, TNF, and myostatin (Mstn), wherein the first RNA transcript and second RNA transcript are not transcribed from the same gene. In some embodiments, the first siRNA and second siRNA are linked via a linking molecule. In some embodiment, the first siRNA is selected from an siRNA set forth in any one of Tables 1-5. In some embodiments, the second siRNA is selected from an siRNA set forth in any one of Tables 1-5.

In some embodiments, a bi-specific nucleic acid molecule (e.g., bispecific siRNA) comprises one siRNA specifically targets ACVR1^{R206H} and a second siRNA that specifically targets INHBA, MSTN, IL-1β, or TNF. In some embodiments, any one of the ACVR1^{R206H} specific siRNA described herein can be combined with any one of the INHBA specific siRNA or MSTN specific siRNA described herein. In some embodiments, a bispecific siRNA described herein is divalent. In some embodiments, a bispecific siRNA described herein comprises a chemically modified siRNA scaffold. In some embodiments, a bispecific siRNA described herein targets muscle cells.

### II. Pharmaceutical Composition

Isolated nucleic acids (e.g., siRNAs) provided herein may be formulated in any suitable manner. Generally, isolated nucleic acids (e.g., siRNAs) provided herein are formulated in a manner suitable for pharmaceutical use as described herein. For example, isolated nucleic acids (e.g., siRNAs) can be delivered to a subject using a formulation that minimizes degradation, facilitates delivery and/or (e.g., and) uptake, and/or (e.g., and) provides another beneficial property to the isolated nucleic acids (e.g., siRNAs) in the formulation. In some embodiments, provided herein are compositions comprising isolated nucleic acids (e.g., siRNAs described herein) and pharmaceutically acceptable excipients. Such compositions can be suitably formulated such that when administered to a subject, either into the immediate environment of a target cell (e.g., cell of the central nervous system, neuron, glial cell, astrocyte, oligodendrocyte, microglial cell and ependymal cell), or systemically, a sufficient amount of the isolated nucleic acids (e.g., siRNAs) enter target cells (e.g., muscle cells). In some embodiments, isolated nucleic acids (e.g., siRNAs described herein) are formulated in buffered solutions, such as phosphate-buffered saline solutions, liposomes, micellar structures, and capsids, for example as described in U.S. Pat. Nos. 9,950,068, 9,050,373, 9,314,529, 8,877,237, US Patent Application Publication No. 20200085974 and US Patent Application Publication No. 20110274745, all of which are herein incorporated by reference. It should be appreciated that, in some embodiments, compositions may include one or more types of nucleic acids provided herein (e.g., siRNAs targeting ACVR1^{R206H}, INHBA, or MSTN).

In some embodiments, isolated nucleic acids (e.g., siRNAs described herein) are formulated in water or in an aqueous solution (e.g., water with pH adjustments). In some embodiments, isolated nucleic acids (e.g., siRNAs described herein) are formulated in a basic buffered aqueous solution (e.g., PBS) with a pH of 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, or 13.9. In some embodiments, formulations as disclosed herein comprise an excipient. In some embodiments, an excipient confers to a composition improved stability, improved absorption, improved solubility, and/or (e.g., and) therapeutic enhancement of the inhibitory nucleic acid. In some embodiments, an excipient is a buffering agent (e.g., sodium citrate, sodium phosphate, a tris base, or sodium hydroxide), a sugar (e.g., lactose, sucrose, mannitol or sorbitol), a vehicle (e.g., a buffered solution, petrolatum, dimethyl sulfoxide, alcohol, or mineral oil, micelle, liposome, capsid, lipid nanoparticle), a salt, a cellulose preparation, a polymer, a lipid, an antioxidant, a preservative, a calcium phosphate, a binder (e.g., starch, gelatin, methyl cellulose, hydroxypropyl methylcellulose, sodium carboxymethylcellulose, and/or polyvinyl pyrrolidone), or any combination thereof.

In some embodiments, isolated nucleic acids (e.g., siRNAs described herein) is lyophilized for extending its shelf-life and then made into a solution before use (e.g., before administration to a subject). Accordingly, an excipient in a composition comprising isolated nucleic acids (e.g., siRNAs described herein) described herein may be a lyoprotectant (e.g., mannitol, lactose, polyethylene glycol, or polyvinyl pyrolidone) or a collapse temperature modifier (e.g., dextran, ficoll, or gelatin).

In some embodiments, a pharmaceutical composition is formulated to be compatible with its intended route of administration. As used herein, the terms “administering” or “administration” means to provide isolated nucleic acids (e.g., siRNAs described herein) to a subject in a manner that is physiologically and/or (e.g., and) pharmacologically useful (e.g., to treat a condition in the subject). Examples of routes of administration include parenteral, e.g., intravenous, intradermal, subcutaneous, administration, or direct administration to the CNS (e.g., intracerebral injection, intraventricular injection, intracisternal injection, intraparenchymal injection, intrathecal injection, and any combination of the foregoing). Pharmaceutical compositions suitable for injectable use include sterile aqueous solutions (where water soluble) or dispersions and sterile powders for the instant preparation of sterile injectable solutions or dispersions. The excipient can be a solvent or dispersion medium containing, for example, water, ethanol, polyol (for example, glycerol, propylene glycol, liquid polyethylene glycol, and the like), and suitable mixtures thereof. In some embodiments, formulations include isotonic agents, for example, sugars, polyalcohols such as mannitol, sorbitol, and sodium chloride, in the composition. Sterile injectable solutions can be prepared by incorporating the isolated nucleic acids (e.g., siRNAs described herein) in a required amount in a selected solvent with one or a combination of ingredients enumerated above, as required, followed by filtered sterilization.

In some embodiments, a composition may contain at least about 0.1%, at least about 0.5%, at least about 1%, at least about 2%, at least about 3%, at least about 4%, at least about 5%, at least about 10%, at least about 12%, at least about 15%, at least about 18%, or at least about 20% isolated nucleic acids (e.g., siRNAs described herein) described herein, or more, although the percentage of the isolated nucleic acids (e.g., siRNAs described herein) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### III. Therapeutic Applications

Isolated nucleic acids (e.g., ACVRR^{206H} specific siRNAs) described herein are effective in treating, preventing, slowing the progression and/or reversing FOP in a subject having, suspected of having FOP, or susceptible of developing FOP (e.g., carrying the ACVRR^{206H} allele). In some embodiments, bispecific siRNAs comprising an ACVRR^{206H} specific siRNAs and a siRNA targeting INHBA, MSTN, TNF, or IL-1β can also be used to treat or prevent FOP. In some embodiments, the present disclosure provides methods for treating FOP, the methods comprising administering a subject having FOP an ACVRR^{206H} specific siRNA described herein. In some embodiments, the present disclosure provides methods for treating FOP, the methods comprising administering a subject having FOP a bi-specific siRNA described herein.

In some embodiments, ACVRR^{206H} specific siRNAs are effective in treating FOP. In some embodiments, ACVRR^{206H} specific siRNAs are effective in preventing, slowing the progression and/or reversing FOP.

In some embodiments, a subject may be a human subject, a non-human primate subject, a rodent subject, or any other suitable mammalian subject. In some embodiments, a subject may have FOP. In some embodiments, a subject is suffering from one or more symptoms of FOP, e.g., bone forming on muscles, ligaments and connective tissue, decreased mobility (scooting instead of crawling, joint stiffness, locked joints), difficulty eating or speaking, hearing impairment, malformed big toe, and/or permanent immobility. In some embodiments, a subject is not yet suffering from any symptoms of FOP.

An aspect of the disclosure includes a method involving administering to a subject an effective amount of ACVRR^{206H} specific siRNAs or the bispecific siRNA as described herein. In some embodiments, an effective amount of a pharmaceutical composition that comprises an ACVRR^{206H} specific siRNAs or the bispecific siRNA can be administered to a subject in need of treatment. In some embodiments, a pharmaceutical composition comprising ACVRR^{206H} specific siRNAs or the bispecific siRNA as described herein may be administered by a suitable route, which may include intravenous administration, e.g., as a bolus or by continuous infusion over a period of time, or by direct injection into the target tissue (e.g., muscle). In some embodiments, a pharmaceutical composition may be in solid form, aqueous form, or a liquid form. In some embodiments, an aqueous or liquid form may be lyophilized. In some embodiments, a lyophilized form may be reconstituted with an aqueous or liquid solution.

Compositions for intravenous administration may contain various carriers, such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble inhibitory nucleic acids can be administered by the drip method, whereby a pharmaceutical formulation containing the inhibitory nucleic acids and physiologically acceptable excipients is infused. Physiologically acceptable excipients may include, for example, 5% dextrose, 0.9% saline, Ringer's solution or other suitable excipients. Intramuscular preparations, can be dissolved and administered in a pharmaceutical excipient such as Water-for-Injection, 0.9% saline, or 5% glucose solution.

In some embodiments, a pharmaceutical composition that comprises ACVRR^{206H} specific siRNAs or the bispecific siRNA is administered via site-specific or local delivery techniques, e.g., direct injection into the target tissue (e.g., muscle).

In some embodiments, a pharmaceutical composition that comprises ACVRR^{206H} specific siRNAs or the bispecific siRNA is administered at an effective concentration that confers a therapeutic effect on the subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the ACVRR^{206H} specific siRNAs or the bispecific siRNA in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of symptoms associated with FOP, through measures of a subject's self-reported outcomes, e.g. mobility, self-care, usual activities, pain/discomfort, and anxiety/depression, or other wellness and quality-of-life indicators.

In some embodiments, a pharmaceutical composition that comprises ACVRR^{206H} specific siRNAs or the bispecific siRNA described herein is administered to a subject at an effective concentration sufficient to inhibit the activity or expression of a target gene (e.g., ACVR1^{R206H}) by at least 10%, at least 20%, at least 30%, at least 40%, at least 50%, at least 60%, at least 70%, at least 80%, at least 90%, or at least 95% relative to a control, e.g. baseline level of gene expression prior to treatment, or compared to a control population.

In some embodiments, a single dose or administration of a pharmaceutical composition that comprises an ACVR1^{206H} specific siRNA or the bispecific siRNA described herein to a subject is sufficient to inhibit activity or expression of ACVRR^{206H} for at least 1-5, 1-10, 5-15, 10-20, 15-30, 20-40, 25-50, or more days. In some embodiments, a single dose or administration of a pharmaceutical composition that comprises an ACVRR^{206H} specific siRNA or the bispecific siRNA described herein to a subject is sufficient to inhibit the activity or expression of ACVRR^{206H} for at least 1, 2, 3, 4, 5, 6, 7, 8, 9, 10, 11, 12, 15, 20, or 24 weeks. In some embodiments, a single dose or administration of a pharmaceutical composition that comprises an ACVRR^{206H} specific siRNA or the bispecific siRNA described herein to a subject is sufficient to inhibit activity or expression of a target gene for at least 1-5, 1-10, 2-5, 2-10, 4-8, 4-12, 5-10, 5-12, 5-15, 8-12, 8-15, 10-12, 10-15, 10-20, 12-15, 12-20, 15-20, or 15-25 weeks. In some embodiments, a single dose or administration of a pharmaceutical composition that comprises an ACVR1^{206H} specific siRNA or the bispecific siRNA described herein to a subject is sufficient to inhibit the activity or expression of a ACVR1^{206H} for at least 1, 2, 3, 4, 5, or 6 months.

In some embodiments, a pharmaceutical composition may comprise more than one an ACVR1^{206H} specific siRNA or the bispecific siRNA. In some embodiments, a pharmaceutical composition may further comprise any other suitable therapeutic agent for treatment of a subject, e.g., a human subject having FOP. In some embodiments, the other therapeutic agents may enhance or supplement the effectiveness of the ACVR1^{206H} specific siRNA or the bispecific siRNA described herein. In some embodiments, the other therapeutic agents may function to treat a different symptom or disease than the an ACVR1^{206H} specific siRNA or the bispecific siRNA described herein. In some embodiments, the pharmaceutical composition is for use in treating, preventing, slowing the progression and/or reversing HO in a subject having FOP.

### EXAMPLES

Fibrodysplasia ossfficans progressiva (FOP, OMIM 135100) is an ultra-rare genetic disorder with a defining feature of progressive, disabling heterotopic ossification (HO) that forms within skeletal muscle, tendons, ligaments, fascia and aponeuroses. HO develops in childhood and early adulthood during episodic flare-ups that occur spontaneously or are triggered by minor trauma, injury, intramuscular injection or inflammation. The new bone leads to progressive immobility and severe pain. In some cases, patients with FOP have heterozygous, gain-of/function mutations in the bone morphogenetic protein (BMP) type I receptor ACVR1. About 97% of FOP patients harbor a ACVR1R^{206H} mutation (c.617G>A;p.R206H). Recent studies showed that ACVR1R^{206H} confers dysregulated BMP-pSMAD1/5 signaling in response to Activin A, which normally inhibits BMP signaling through the wild-type (WT) ACVR1 receptor.

FOP treatment is challenging due to the early age of onset of ectopic bone formation and the difficulty in selectively suppressing BMP signaling by the ACVR1^{R206H} mutation. Presently, there is no definitive prevention or treatment for the progressively disabling HO except for symptomatic management with high-dose corticosteroids for episodic flare-ups.

For FOP patients harboring a heterozygous, gain-of-function ACVR1^{R206H} mutation (c.617G>A;p.R206H), selective silencing of ACVR1^{R206H} allele via single nucleotide polymorphism (SNP) heterozygosity would be effective in treating FOP. Using a fully chemically modified siRNA scaffold, we developed siRNA that can selectively silence human ACVR1R^{206H} allele, but not the wild type allele, in human FOP-patient derived induced pluripotent stem cells (iPSCs) and mouse bone marrow-derived stromal cells (BMSCs) expressing human ACVR1^{R206H} receptor.

For efficient, sustainable, sequence-specific gene silencing in skeletal muscles without any induction of pro-inflammatory cytokines, ACVR1^{R206H} allele-specific siRNA was conjugated to DCA. Further, by using a divalent, chemically modified siRNA scaffold, a muscle targeting, bi-specific siRNAs that enables ACVR1^{R206H} allele-specific silencing and Activin A silencing simultaneously were also developed.

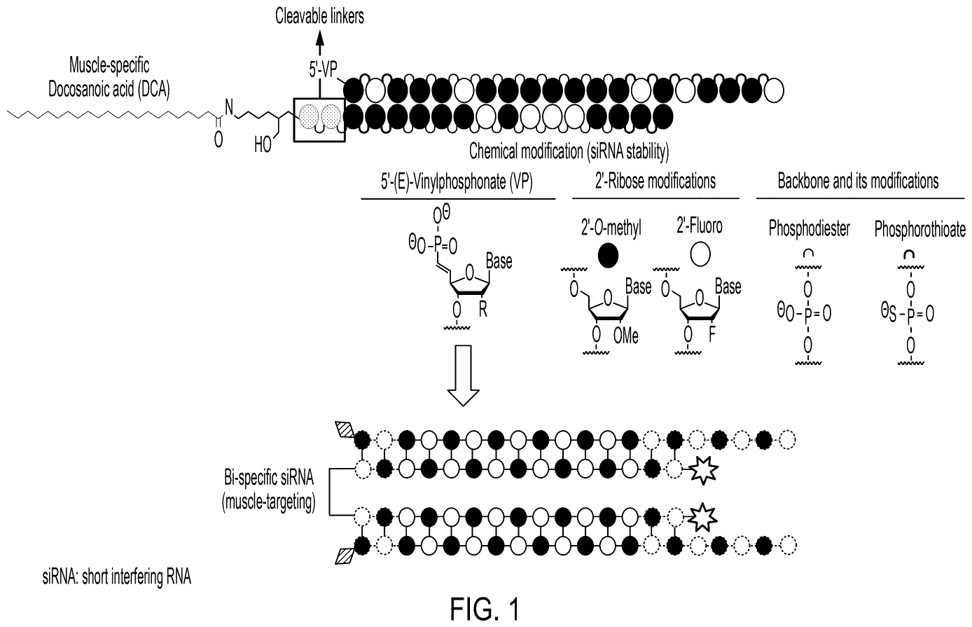

The following examples describe development of chemically-modified, muscle-targeting siRNAs for silencing of genes associated with FOP, for example ACVR1 and Inhba. FIG. 1 provides a schematic depicting structural features of siRNAs and bi-specific siRNA molecules for use in treatment of FOP.

### Example 1

This example describes development of chemically-modified siRNAs that silence ACVR^{R206H} in an allele-specific manner. A panel of sixteen chemically-stabilized siRNAs overlapping single nucleotide polymorphisms (SNPs) of human ACVR1^{R206H} were produced, as described in Table 1. The siRNAs contained an alternating pattern of 2′-O-methyl (2′-OMe) and deoxy-2′-fluoro (2′-F) ribose modifications and terminal backbone modifications with phosphorothioate (PS); the sense strand of the siRNA was conjugated to cholesterol. At least one sequence mismatch (e.g., SNP) of ACVR1^{R206H} and wild-type ACVR1 was included in each siRNA (Table 2).

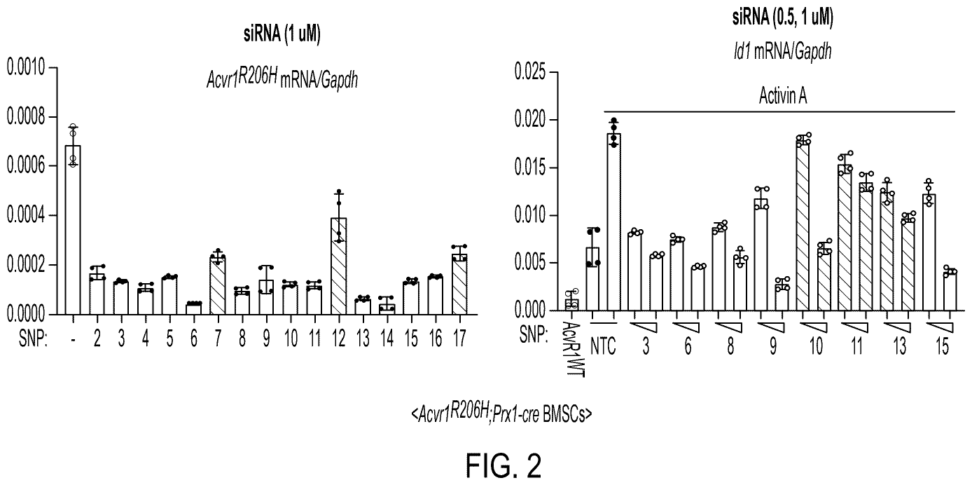

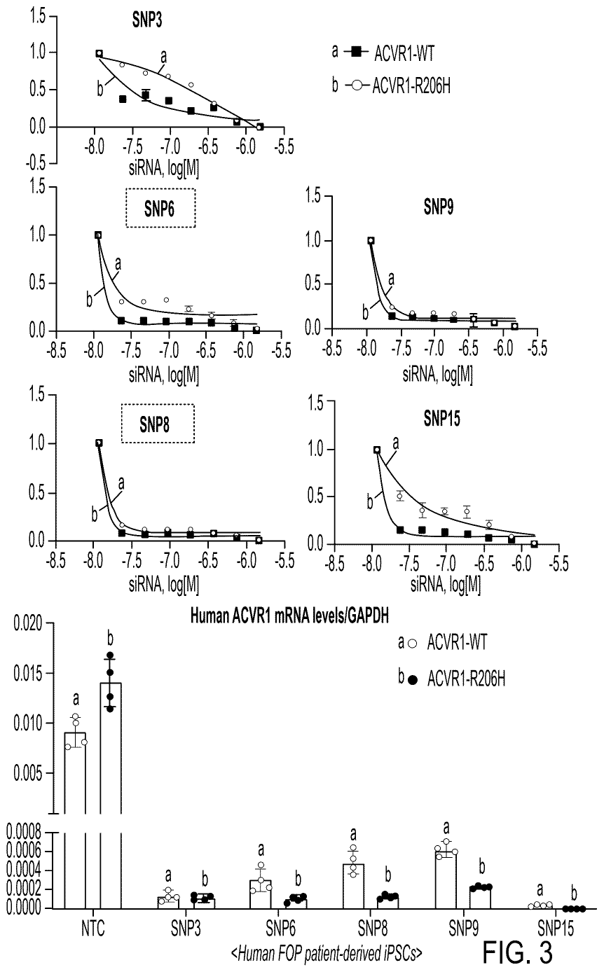

The siRNAs were screened using mouse BMSCs expressing human ACVR1^{R206H} receptor. Cells were treated with siRNAs for two days and knockdown efficiency was assessed by qPCR. All but two of the tested siRNAs were highly effective in silencing human ACVR1^{R206H} expression. A moderate to significant decrease in Activin A-induced expression of Msx2 was also observed for several of the siRNAs (SNP2, 3, 4, 5, 6, 8, 9, 10, 11, 13, 15, and 16). Additionally, SNP3, SNP6, SNP8, SNP9, and SNP15 were observed to be effective in silencing both ACVR1^{R206H} expression and aberrant Activin A signaling in FOP BMSCs (FIG. 2).

The ability of the siRNAs to silence ACVR1^{R206H} in an allele-specific manner was investigated using FOP patient-derived iPSCs heterozygous for the ACVR1^{R206H} allele. SNP6, SNP8, and SNP9 were observed to silence ACVR1^{R206H} moderately more than wild-type ACVR1 (FIG. 3). SNP-based allele-specific silencing of ACVR1^{R206H} was also tested using a luciferase sensor system. 293T cells were transfected with a sensor plasmid and treated with siRNAs in a 7-point dose response curve, and luciferase activity was measured. The siRNAs having SNP in positions 6, 8, 9, and 15 were observed to silence ACVR1^{R206H} with discrimination between ACVR1 alleles.

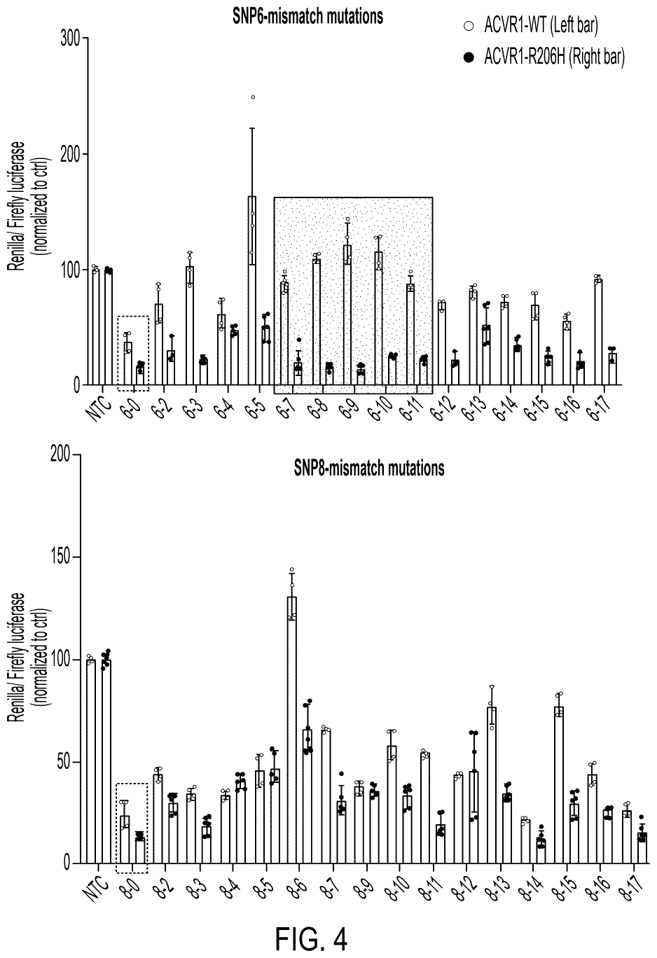

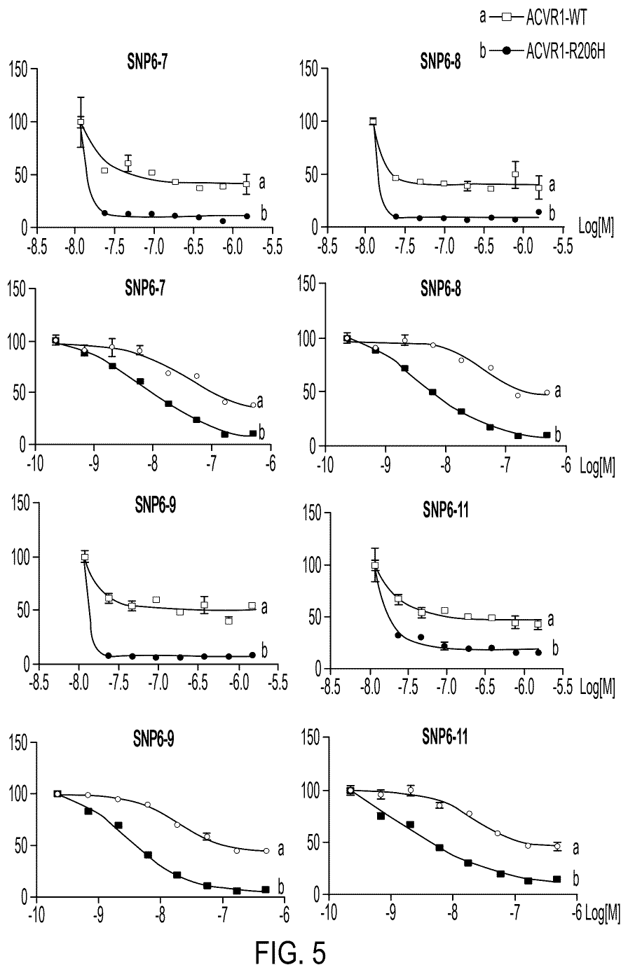

In order to improve allele discrimination, siRNAs having two SNP mismatches to human wild-type ACVR1 and one mismatch to ACVR1^{R206H} were tested, as shown in Table 2. SNP6 and SNP8 were further modified to include an additional mismatch to wild-type ACVR1 at position 7, 8, 9, or 11, and evaluated in a seven-point dose response curve (FIG. 4). Data indicate that siRNAs having mismatches at positions 6 and 9 (SNP6-9) showed the highest discrimination between ACVR1 alleles, increasing greater than 50-fold relative to controls (FIG. 5)

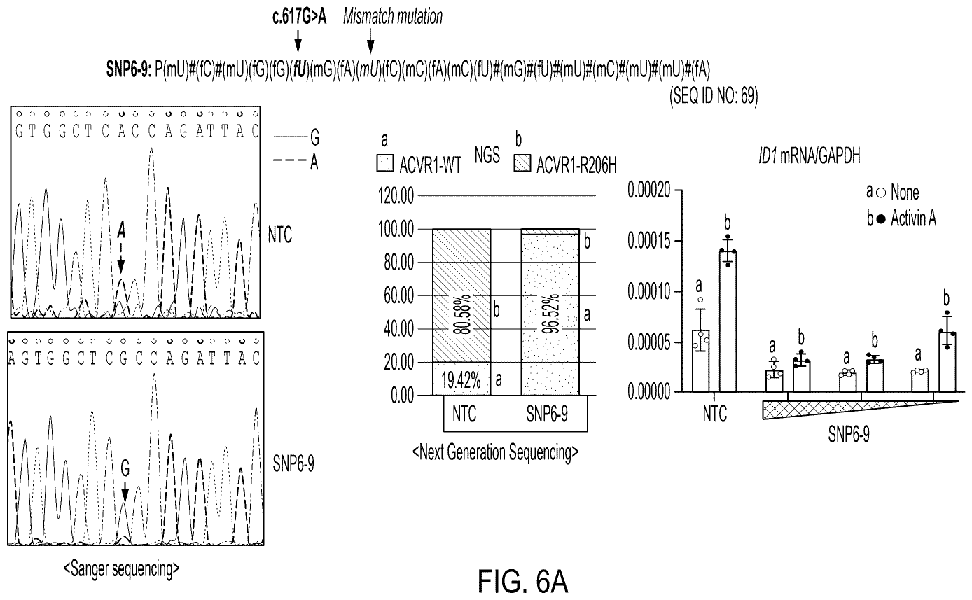

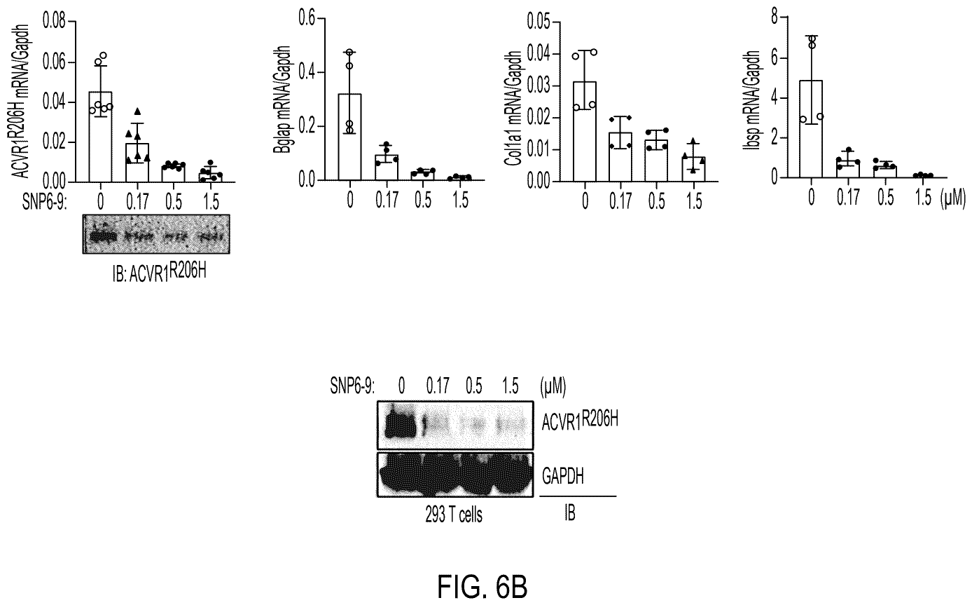

To determine whether SNP6-9 could discriminate between wild-type ACVR1 and ACVR1^{R206H} mRNA in a native human context, human FOP patient-derived iPSCs were treated with a control or SNP6-9 siRNA and samples were subjected to Sanger sequencing or next-generation sequencing (NGS) analysis. Sanger sequencing data indicates a shift from c.617A to c.617G in FOP iPSCs when treated with SNP-6-9. This is consistent with NGS analysis of SNP6-9 treated cells showing a significant decrease in ACVR1^{R206H} ratios (3.48%) and an increase in wild-type ACVR1 ratios (96.52%), compared to NTC-treated FOP cells showing 80.58% ACVR1^{206H} vs. 19.42% wild-type ACVR1 (FIG. 6A). Notably, reduced expression of ACVR1^{206H} receptor in SNP6-9 treated cells blunted Activin A-induced expression of Id1, indicating that SNP6-9 siRNA is highly-selective in silencing ACVR1^{R206H} expression and potent to suppress aberrant Activin A signaling in human FOP cells.

To examine inhibitory effects of SNP6-9 on osteogenic differentiation of FOP cells, ACVR1^{R206H/prx1} BMSCs were cultured under osteogenic conditions in the presence of SNP6-9. mRNA and protein levels of ACVR1^{R206H} were examined by qPCR and immunoblotting analyses, demonstrating dose-responsive knockdown of human ACVR1^{R206H} protein expression (FIG. 6B). Likewise, SNP6-9 treatment abolished human ACVR1^{R206H} protein expression in 293T cells (FIG. 6B). This is accompanied with a dose-responsive decrease in the expression of osteogenic genes, including osteocalcin (Balp), type 1 collagen alpha-1 (Col1a1), and bone sialoprotein (Ibsp), indicating that SNP6-9 is a potent suppressor of ACVR1R^{206H}-induced osteogenesis in FOP BMSCs.

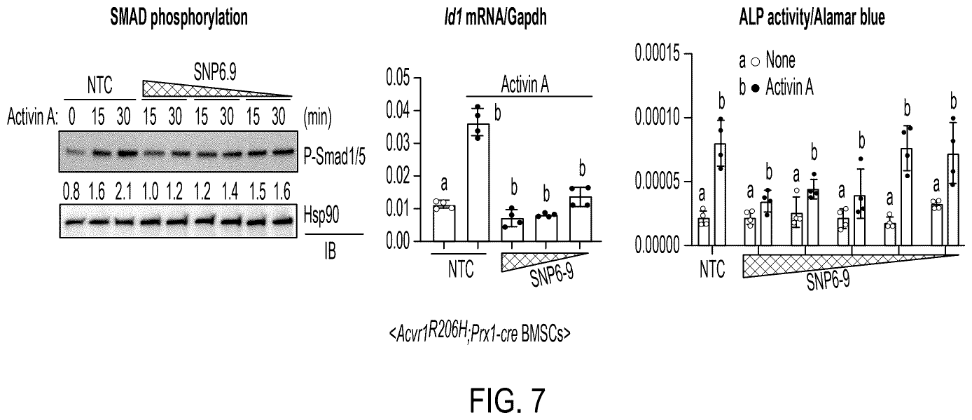

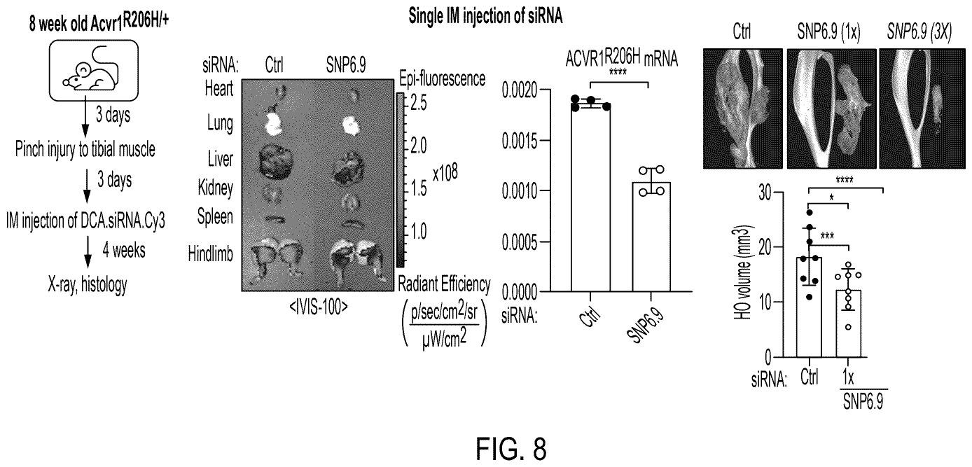

Given that the ACVR1^{R206H} mutation activates Smad1/5-mediated BMP pathway signaling in response to Activin A, the ability of SNP6-9 to suppress Activin A signaling in ACVR1R^{206H/prx1} BMSCs was investigated. Smad1/5 phosphorylation in NTC-treated cells occurred at 15 minutes or 30 minutes after Activin A stimulation, but this phosphorylation was markedly reduced in the presence of SNP6-9 (FIG. 7). Additionally, SNP6-9 treatment markedly reduced Activin A-induced Id1 expression, alkaline phosphatase activity (an early osteogenic marker) and mineral deposit (a late osteogenic marker) in a dose-dependent manner. A single IM injection of SNP6-9 conjugated to docosanoic acid (DCA) was also observed to reduce HO in vivo in a mouse model of FOP (FIG. 8).

### Example 2

This example describes development of chemically-modified siRNAs that silence mouse or human Inhba. A panel chemically-stabilized siRNAs overlapping single nucleotide polymorphisms (SNPs) of human Inhba were produced, as described in Table 3. The siRNAs contained an alternating pattern of 2′-O-methyl (2′-OMe) and deoxy-2′-fluoro (2′-F) ribose modifications and terminal backbone modifications with phosphorothioates (PS); the sense strand of the siRNA was conjugated to cholesterol.

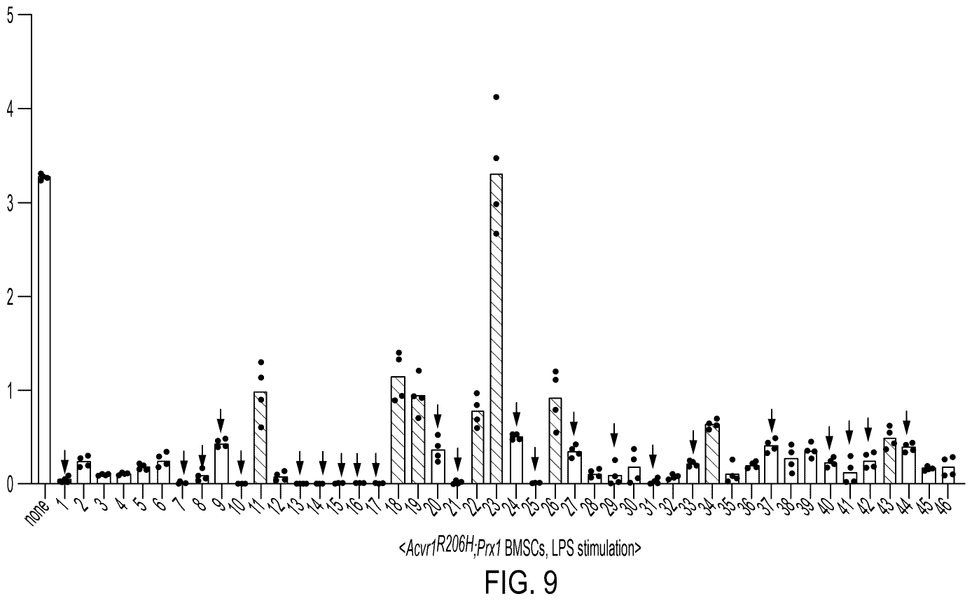

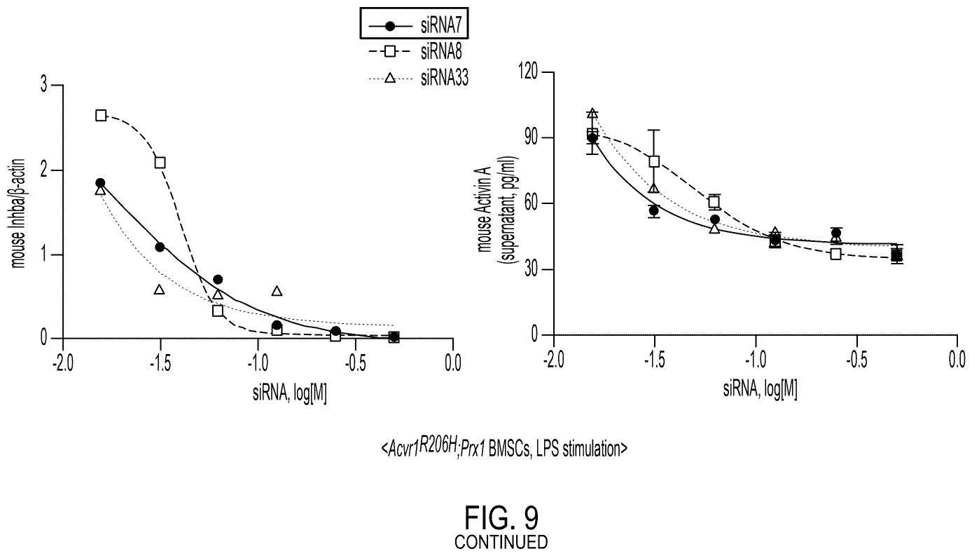

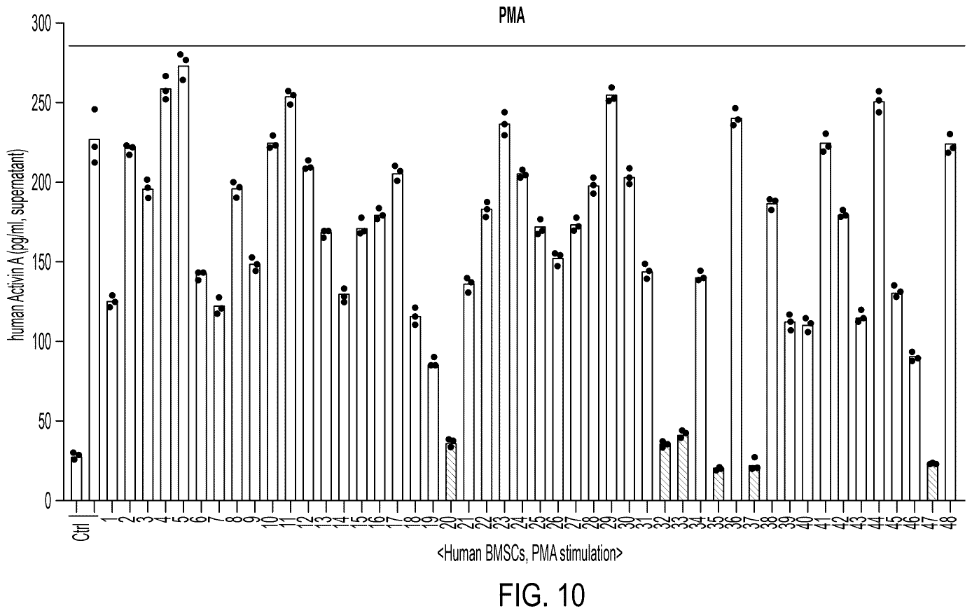

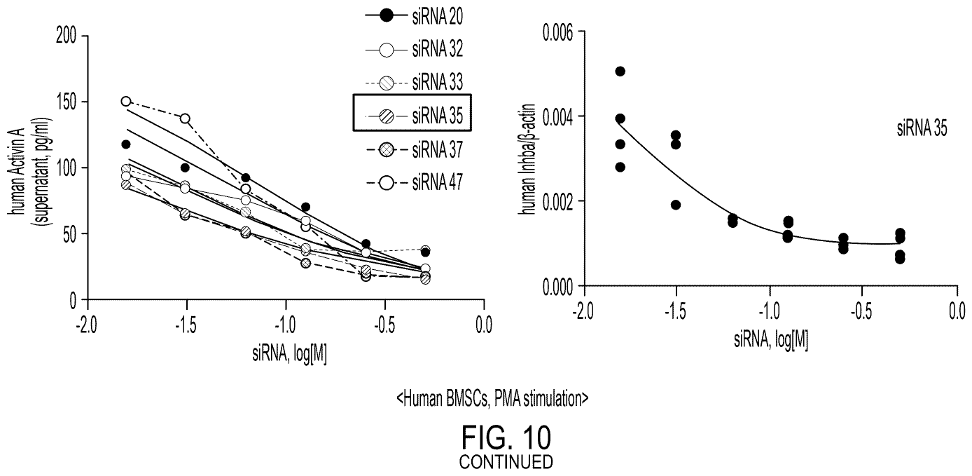

The siRNAs were screened in mouse ACVR^{R206H/prx1} BMSCs or human BMSCs, using qPCR, ELISA and immunoblotting analyses. Cells were treated for two days with 0.5 μM siRNA, stimulated with LPS for 8 hours, and mRNA levels of Inhba were measured. Secreted Activin A protein in the cell supernatant was also measured, and intracellular expression of Inhba protein was confirmed by ELISA (FIG. 9). Three mouse Inhba-targeting siRNAs were selected for further investigation in a seven-point dose response curve. siRNA7 was identified as being the most effective siRNA for silencing mouse Inhba (FIG. 9). Six human Inhba-targeting siRNAs were also selected for further investigation in a seven-point dose response curve. siRNA35 was identified as being the most effective siRNA for silencing human Inhba (FIG. 10).

### Example 3

This example describes in vivo silencing of ACVR1^{R206H} using DCA-conjugated siRNAs. Fluorescently-labeled siRNA SNP6-9 was intramuscularly injected into a mouse model of FOP with injury-induced HO three days after muscle injury. Four weeks later, siRNA expression in individual tissues was measured using an optical imaging system. Data indicate that siRNA-treated mice showed strong fluorescence in skeletal muscle and moderate expression in liver (FIG. 8). SNP6-9 treatment markedly reduced mRNA levels of ACVR1^{R206H}, and micro CT analysis showed a significant decrease in heterotopic bone mass in treated muscle (FIG. 8), indicating local delivery of SNP6-9 suppresses trauma-induced HO in vivo.

### Example 4

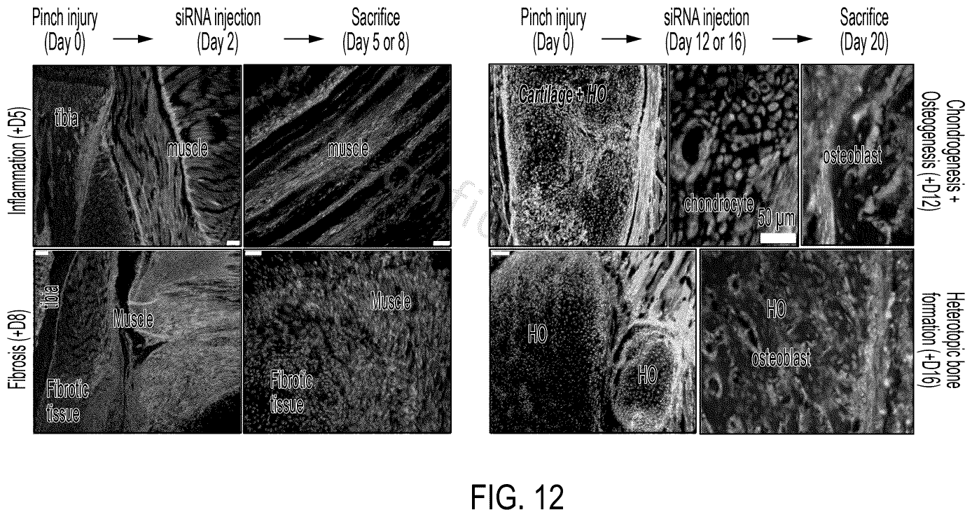

This example describes conjugation of docosanoic acid (DCA) to siRNAs in order to improve muscle tissue targeting. To examine the ability of DCA-conjugated siRNAs to transfect skeletal muscle, where HO primarily develops in FOP mice, fluorescently-tagged DCA-siRNAs were administered to a mouse model of FOP by intramuscular injection. Injured muscle tissue was then harvested and analyzed at serial timepoints to assess siRNA distribution during HI pathogenesis using fluorescence microscopy. Data indicate that at 3- and 6-days post-injury, fluorescence was detected in damaged muscle fibers and infiltrated immunes cells but not in highly-proliferative fibrotic tissues. Likewise, there was little to no fluorescence in the cartilage analgen and heterotopic bone in the HO lesions 12-16 days post-injury, indicating fluorescence in these cells may disappear due to dilution of siRNAs in highly proliferating cells (FIG. 12). siRNA were also administered to the mice 12 and 16-days post-injury, where cartilage analgen and heterotopic bone was fully developed (FIG. 12). Data indicate that muscle fibers, chondrocytes within the cartilage analgen, osteoblasts, and osteocytes within heterotopic bone were all transfected with the DCA-siRNAs, demonstrating high transfection efficiency in HO-causing cell types (e.g., immune cells, chondrocytes, myoblasts/myocytes, etc.), when locally administered.

### Example 5

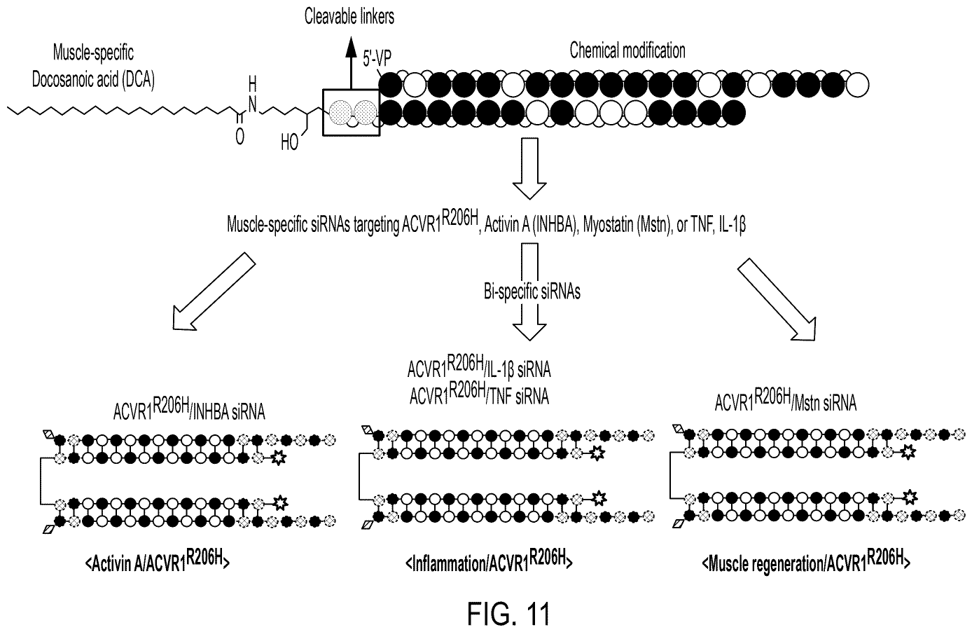

This example describes bi-specific siRNAs targeting genes associated with FOP. In some embodiments, the bi-specific siRNAs comprise a first chemically-modified siRNA targeting a gene selected from ACVR1^{R206H}, Inhba, IL-1β, TNF, and myostatin (Mstn), linked to a second chemically-modified siRNA targeting a gene selected from ACVR1^{R206H}, Inhba, IL-1β, TNF, and myostatin (Mstn), where the first and second siRNAs do not target the same gene. In some embodiments, the bi-specific siRNAs are conjugated to DCA, for example by a cleavable linker. Representative structures of the bi-specific siRNAs are shown in FIG. 11. Examples of siRNAs targeting Mstn are shown in Tables 4 and 5.

1. An isolated nucleic acid comprising a sense strand and an antisense strand, wherein the antisense strand comprises a sequence that is at least 80% complementary to at least 18 consecutive nucleotides of an activin receptor type 1 (ACVR1) R206H (ACVR1^{R206H}) sequence of SEQ ID NO: 675.

2. (canceled)

3. The isolated nucleic acid of claim 1, wherein:
the antisense strand is at least 80% complementary to a sequence as set forth in any one of SEQ ID NOs: 676-721;
the antisense strand comprises a nucleic acid sequence of any one of SEQ ID NOs: 768-813; and/or
the sense strand comprises a nucleic acid sequence of any one of SEQ ID NOs: 722-767.

4-14. (canceled)

15. The isolated nucleic acid of claim 1, wherein:
the sense strand comprises a chemical modification pattern set forth in Table 1 or 2;
the antisense strand comprises a chemical modification pattern set forth in Table 1 or 2;
the sense strand comprises a sequence set forth in any one of SEQ ID NOs: 1-16, and 33-62; and/or
the antisense strand comprises a sequence set forth in any one of SEQ ID NOs: 17-32 and 63-92.

16-18. (canceled)

19. An isolated nucleic acid comprising a sense strand and an antisense strand, wherein the antisense strand comprises a sequence that is at least 80% complementary to at least 18 consecutive nucleotides of an inhibitin beta A (INHBA) sequence of any one of SEQ ID NOs: 817-912.

20. (canceled)

21. The isolated nucleic acid of claim 19, wherein:
the antisense strand is at least 80% complementary to a sequence as set forth in any one of SEQ ID NOs: 913-1007;
the antisense strand comprises a nucleic acid sequence of any one of SEQ ID NOs: 1008-1103; and/or
the sense strand comprises a nucleic acid sequence of any one of SEQ ID NOs: 1104-1199.

22-32. (canceled)

33. The isolated nucleic acid of claim 19, wherein:
the sense strand comprises a chemical modification pattern set forth in Table 3;
the antisense strand comprises a chemical modification pattern set forth in Table 3;
the sense strand comprises a sequence set forth in any one of SEQ ID NOs: 93-188; and/or
the antisense strand comprises a sequence set forth in any one of SEQ ID NOs: 189-284.

34-36. (canceled)

37. An isolated nucleic acid comprising a sense strand and an antisense strand, wherein the antisense strand comprises a sequence that is at least 80% complementary to at least 18 consecutive nucleotides of a myostatin (MSTN) sequence of any one of SEQ ID NOs: 1376-1472 and 1200-1287.

38. (canceled)

39. The isolated nucleic acid of claim 37, wherein:
the antisense strand is at least 80% complementary to a sequence as set forth in any one of SEQ ID NOs: 1473-1569 and 1288-1375;
the antisense strand comprises a nucleic acid sequence of any one of SEQ ID NOs: 1570-1657 and 1746-1842; and/or
the sense strand comprises a nucleic acid sequence of any one of SEQ ID NOs: 1658-1745 and 1843-1939.

40-50. (canceled)

51. The isolated nucleic acid of claim 37, wherein:
the sense strand comprises a chemical modification pattern set forth in Table 4 or 5;
the antisense strand comprises a chemical modification pattern set forth in Table 4 or 5;
the sense strand comprises a sequence set forth in any one of SEQ ID NOs: 285-372 and 481-577; and/or
the antisense strand comprises a sequence set forth in any one of SEQ ID NOs: 373-480 and 578-674.

52-54. (canceled)

55. A bi-specific nucleic acid molecule comprising:
(i) a first siRNA targeting a first RNA transcript selected from activin receptor type 1 (ACVR1) R206H (ACVR1^{R206H}), INHBA, IL-1β, TNF, and myostatin (MSTN); linked to
(ii) a second siRNA targeting a second RNA transcript selected from ACVR1^{R206H}, INHBA, IL-1β, TNF, and myostatin (MSTN),
wherein the first RNA transcript and the second RNA transcript are not transcribed from a same gene.

56. (canceled)

57. The bi-specific nucleic acid molecule of claim 55, wherein:
the first siRNA is selected from an siRNA set forth in any one of Tables 1-5; and/or
the second siRNA is selected from an siRNA set forth in any one of Tables 1-5.

58-60. (canceled)

61. A composition comprising the isolated nucleic acid of claim 1 and a pharmaceutically acceptable excipient.

62. A method for inhibiting Activin A activity or function or treating or preventing heterotopic ossification (HO) or fibrodysplasia ossificans progressiva (FOP) in a subject in need thereof, the method comprising administering to the subject the isolated nucleic acid of claim 1.

63. A method for inhibiting Activin A activity or function or treating or preventing heterotopic ossification (HO) or fibrodysplasia ossificans progressiva (FOP) in a subject in need thereof, the method comprising administering to the subject the isolated nucleic acid of claim 19.

64. A method for inhibiting Activin A activity or function or treating or preventing heterotopic ossification (HO) or fibrodysplasia ossificans progressiva (FOP) in a subject in need thereof, the method comprising administering to the subject the isolated nucleic acid of claim 37.

65. (canceled)

66. The method of claim 62, wherein the subject is a human and comprises one or more mutations in an ACVR1 allele.

67. A composition comprising the isolated nucleic acid of claim 19 and a pharmaceutically acceptable excipient.

68. A composition comprising the isolated nucleic acid of claim 37 and a pharmaceutically acceptable excipient.

69. A composition comprising the bi-specific nucleic acid molecule of claim 55 and a pharmaceutically acceptable excipient.

70. A method for inhibiting Activin A activity or function or treating or preventing heterotopic ossification (HO) or fibrodysplasia ossificans progressiva (FOP) in a subject in need thereof, the method comprising administering to the subject the bi-specific nucleic acid molecule of claim 55.

71. The method of claim 70, wherein the subject is a human and comprises one or more mutations in an ACVR1 allele.

72. A method for inhibiting Activin A activity or function or treating or preventing heterotopic ossification (HO) or fibrodysplasia ossificans progressiva (FOP) in a subject in need thereof, the method comprising administering to the subject a composition comprising the isolated nucleic acid of claim 1 and a pharmaceutically acceptable excipient.

73. A method for inhibiting Activin A activity or function or treating or preventing heterotopic ossification (HO) or fibrodysplasia ossificans progressiva (FOP) in a subject in need thereof, the method comprising administering to the subject a composition comprising the isolated nucleic acid of claim 19 and a pharmaceutically acceptable excipient.

74. A method for inhibiting Activin A activity or function or treating or preventing heterotopic ossification (HO) or fibrodysplasia ossificans progressiva (FOP) in a subject in need thereof, the method comprising administering to the subject a composition comprising the isolated nucleic acid of claim 37 and a pharmaceutically acceptable excipient.

75. A method for inhibiting Activin A activity or function or treating or preventing heterotopic ossification (HO) or fibrodysplasia ossificans progressiva (FOP) in a subject in need thereof, the method comprising administering to the subject a composition comprising the bi-specific nucleic acid molecule of claim 55 and a pharmaceutically acceptable excipient.
